(19) United States
(12) Patent Application Publication
(10) Pub. No.: US 2010/0247461 A1
(43) Pub. Date: Sep. 30, 2010

(54) ANTI-INFLAMMATORY COMPLEXES

(75) Inventors: Michael Voronkov, Pennington, NJ (US); Jeffry B. Stock, Princeton, NJ (US); Maxwell Stock, Rocky Hill, NJ (US); Seung-Yub Lee, Princeton, NJ (US); Eduardo Perez, Somerset, NJ (US); Joel S. Gordon, Princeton Junction, NJ (US)

Correspondence Address: CHOATE, HALL & STEWART LLP, TWO INTERNATIONAL PLACE, BOSTON, MA, 02110, US

(73) Assignee: Signum Biosciences, Inc., Monmouth Junction, NJ (US)

(21) Appl. No.: 12/690,906

(22) Filed: Jan. 20, 2010

Related U.S. Application Data
(60) Provisional application No. 61/145,887, filed on Jan. 20, 2009.

(51) Int. Cl. A61K 31/465; C07C 323/59; A61K 31/198; C07C 323/58; C07H 5/06; A61K 31/7008; C07D 213/82; A01N 43/40; A01N 43/16; A01N 55/02; A01P 1/00; A61K 31/315; C07F 3/06; C07F 7/28; A61K 31/28; C07F 1/10; A61P 31/04; A61P 17/00; A61P 17/02; A61P 17/10; A61P 11/00; A61P 15/02; A61P 11/06; A61Q 17/04; A61K 8/60; A61K 8/58; A61P 39/06; C12N 5/02

(52) U.S. Cl. 424/59; 562/557; 514/562; 536/55.2; 514/62; 546/316; 514/355; 514/494; 556/118; 556/51; 514/492; 556/110; 514/495; 435/375

(57) ABSTRACT

Disclosed are certain complexes of AFC compounds and binding agents. Such complexes are useful, among other things, in the treatment of inflammatory diseases or disorders.

### RELATED APPLICATIONS

This application claims priority to U.S. provisional patent application Ser. No. 61/145,887, filed Jan. 20, 2009, the entire disclosure of which is incorporated herein by reference.

### BACKGROUND

Inflammation often is a bodily response to infection or injury in which cells involved in detoxification and repair are mobilized to the compromised site by inflammatory mediators. Such infection or injury can be a result of acute or chronic disease, disorders, conditions or trauma, or of environmental conditions or aging. Examples include diseases, disorders, syndromes, conditions and injuries of the cardiovascular, digestive, integumentary, muscular, nervous, reproductive, respiratory and urinary systems, as well as, diseases, disorders, syndromes, conditions and injuries of tissue and cartilage such as atherosclerosis, irritable bowel syndrome, psoriasis, tendonitis, Alzheimer's disease and vascular dementia, multiple sclerosis, diabetes, endometriosis, asthma and kidney failure. Treatment of inflammatory diseases or disorders with traditional anti-inflammatory drugs, e.g., corticosteroids and non-steroidal anti-inflammatory drugs (“NSAIDS”) can cause multiple side effects, e.g., appetite and weight gain, excess sweating, high blood pressure, nausea, vomiting, diarrhea, etc.

### SUMMARY

The present invention provides, among other things, complexes for the treatment of inflammatory diseases or disorders.

In certain embodiments, the present invention provides a complex comprising a compound of formula I:

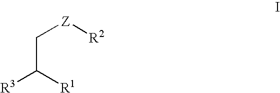

wherein Z, R¹, R², and R³ are defined herein, together with a binding partner.

In some embodiments, the present invention provides a complex comprising a compound of formula II:

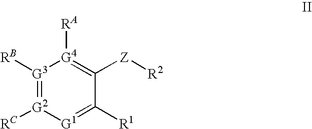

wherein Z, G¹, G², G³, G⁴, R¹, R², R^{A}, R^{B}, and R^{C} are as defined herein, together with a binding partner.

In some embodiments, the present invention provides a complex comprising a compound of formula III:

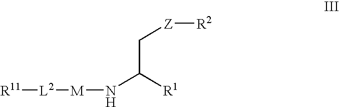

wherein R¹, R², R¹¹, Z, L², and M are as defined herein, together with a binding partner.

In some embodiments, the present invention provides a complex comprising a compound of formula IV:

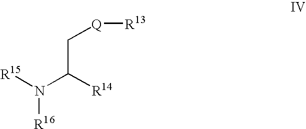

wherein R¹³, R¹⁴, R¹⁵, R¹⁶, and Q are as defined herein, together with a binding partner.

In some embodiments, the present invention provides a complex comprising a compound of formula V:

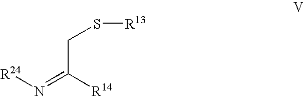

wherein R¹³, R¹⁴, and R²⁴ are as defined herein, together with a binding partner.

In some embodiments, the binding partner and compound of formula I, II, III, IV, or V are present in a ratio within the range of about 0.5:4.5 to 2:1. In some embodiments, the binding partner and compound of formula I, II, III, IV, or V are present in a ratio of about 1:4. In some embodiments, the binding partner and compound of formula I, II, III, IV, or V are present in a ratio of about 1:1. In some embodiments, the binding partner and compound of formula I, II, III, IV, or V are present in a ratio of about 1:2. In some embodiments, the binding partner and compound of formula I, II, III, IV, or V are present in a ratio of about 2:1. In some embodiments, the binding partner and compound of formula I, II, III, IV, or V are present in a ratio of about 1:3. In some embodiments, the binding partner and compound of formula I, II, III, IV, or V are present in a ratio of about 0.5:4.5.

In some embodiments, the present invention provides complexes of AFC compounds and binding partners.

In certain embodiments, treatment of inflammatory diseases or disorders is achieved using complexes of the present invention.

In certain embodiments, treatment of inflammatory diseases or disorders is achieved using provided complexes without having side effects of corticosteroids or NSAIDS.

In certain embodiments, the present invention provides complexes in which the binding partner comprises a metal, a metal isotope, a metal cation, a small molecule containing a basic nitrogen, a topical analgesic, an opiate, a morphinomimetic, a skin whitening agent, an anti-cancer agent and/or an intraocular pressure reducing agent.

The present invention encompasses the finding that certain complexes have superior biological activity relative to other complexes and/or compound (e.g., AFC) alone. For example, the present disclosure illustrates superior activity of certain complexes in one or more biological assays. In some embodiments, superiority is demonstrated as compared with compound not in complexed form and/or as compared with compound in a different complex. For example, in some embodiments, complexes in which the binding partner is strontium show improved MPO (myeloperoxidase) inhibition than do corresponding calcium complexes. In some embodiments, strontium complexes are more effective at decreasing edema than are corresponding calcium complexes and/or are more effective at decreasing sensory irritation than are corresponding calcium complexes.

In certain embodiments, the present invention provides a composition comprising a complex containing a compound of formula I, II, III, IV, or V, a binding partner and at least one pharmaceutically acceptable carrier or excipient.

In certain embodiments, provided complexes are administered in vitro; in certain embodiments such complexes are administered in vivo.

In certain embodiments, such complexes are administered to a subject suffering from or susceptible to one or more inflammatory disorders.

The present invention particularly demonstrates that certain complexes comprising an AFC compound and a binding partner have surprising and desirable characteristics. For example, among other things, according to the present invention, certain such complexes show surprising respiratory burst inhibition, inhibition of edema, and/or inhibition of dermal neutrophil infiltration, for example as measured by inhibition of MPO (myeloperoxidase). The present invention particularly demonstrates surprising attributes of certain such complexes in which the binding partner comprises strontium. The present invention further teaches the surprising desirability of complexes in which the binding partner is glucosamine.

The present invention also demonstrates in some embodiments that complexes containing a particular AFC compound and a binding partner can have similar or better activity in vivo and/or in vitro than the AFC compound alone (e.g., AFC free acid). Without wishing to be bound by any one particular theory, it is believed that provision of a countercharge in a complex may alter the physical properties of the complex as compared with the uncomplexed AFC compound. Alternatively or additionally, it is possible that in some embodiments the binding partner may have anti-inflammatory activity independent of or in addition to any anti-inflammatory activity of the AFC compound. In some such embodiments, the AFC compound may act as a carrier that efficiently delivers an anti-inflammatory agent (e.g., strontium, glucosamine, etc.) to an active site.

### DEFINITIONS

“N-acetyl-S-farnesyl-L-cysteine compound” or an “AFC compound”: As used herein, an “N-acetyl-S-farnesyl-L-cysteine compound” (or an “AFC compound”), as used herein, is a small molecule compound that is structurally related to N-acetyl-S-farnesyl-L-cysteine (AFC). In some embodiments, an AFC compound as provided herein has a structure set forth in any of Formulae I (e.g., Ia, Ib, Ic, Id, Ie, If, Ig), II, III, IV, or V. In some embodiments, an AFC compound may also be referred to as an “isoprenyl compound.” In some embodiments, an AFC compound has the structure of Compound A in FIG. 2 and is referred to as “N-acetyl-S-farnesyl-L-cysteine” or “AFC”. In some embodiments, an AFC compound has the structure of Compound B in FIG. 2 and is referred to as “N-succinyl-S-farnesyl-L-cysteine” or “SFC”. In some embodiments, an AFC compound has the structure of Compound C in FIG. 2 and is referred to as “N-malonyl-S-farnesyl-L-cysteine” or “MFC”.

“Acyl”: As used herein, the term “acyl” refers to a radical formed from an organic acid by removal of a hydroxyl group.

“Additional active ingredient”: As used herein, the phrase “additional active ingredient” refers to an agent, other than an AFC compound that exerts a pharmacological, dermatological or any other beneficial activity. It is to be understood that “other beneficial activity” may be one that is only perceived as such by the subject using the inventive compositions. Typically, an additional active ingredient, as that term is used herein, refers to a pharmaceutically active agent that is administered in combination with an isoprenyl compound of the present invention.

“Aliphatic”: The term “aliphatic”, as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, cycloalkynyl moieties. Thus, as used herein, the term “alkyl” includes straight, branched and cyclic alkyl groups (see below). An analogous convention applies to other generic terms such as “alkenyl”, “alkynyl”, and the like. Furthermore, as used herein, the terms “alkyl”, “alkenyl”, “alkynyl”, and the like encompass both substituted and unsubstituted groups. In some embodiments, an aliphatic group contains 1-25 aliphatic carbon atoms. In some embodiments, an aliphatic group contains from 1 to 25, from 1 to 24, from 1 to 23, from 1 to 22, from 1 to 21, from 1 to 20, from 1 to 19, from 1 to 18, from 1 to 17, from 1 to 16, from 1 to 15, from 1 to 14, from 1 to 13, from 1 to 12, from 1 to 11, from 1 to 10, from 1 to 9, from 1 to 8, from 1 to 7, from 1 to 6, from 1 to 5, from 1 to 4, from 1 to 3, from 1 to 2, from 2 to 3, or 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, 20 to 21, 21 to 22, 22 to 23, 23 to 24, or 24 to 25 aliphatic carbon atoms. In certain embodiments, as used herein, “lower alkyl” is used to indicate those alkyl groups (cyclic, acyclic, substituted, unsubstituted, branched, or unbranched) having 1-6 carbon atoms. In some embodiments, wherein a portion of a term such as alkyl, alkenyl, alkynyl, cycloalkyl, cycloalkenyl, cycloalkynyl is used within a different generic term (e.g., dialkylamino, alkoxy, alkylthio, alkylamino), then it is understood that an analogous convention applies with respect to the number of carbon atoms present.

“Alkenyl”: As used herein, the term “alkenyl” denotes a substituted or unsubstituted, monovalent group derived from a straight- or branched-chain hydrocarbon moiety containing at least one carbon-carbon double bond by removal of a single hydrogen atom. In some embodiments, the alkenyl group contains 1-25 aliphatic carbon atoms. In certain embodiments, an alkenyl group employed in the invention contains 10-25 carbon atoms. In certain embodiments, an alkenyl group employed in the invention contains 10-20 carbon atoms. In certain embodiments, an alkenyl group employed in the invention contains 10-15 carbon atoms. In certain embodiments, an alkenyl group employed contains 10 carbon atoms. In certain embodiments, an alkenyl group employed contains 15 carbon atoms. In certain embodiments, an alkenyl group employed contains 20 carbon atoms. Alkenyl groups include, for example, decenyl, undecenyl, dodecenyl, tridecenyl, tetradecenyl, pentadecenyl, hexadecenyl, heptadecenyl, octadecenyl, nonadecenyl, eicosenyl, heneicosenyl, docosenyl, tricosenyl, tetracosenyl, pentacosenyl, polyunsaturated alkenes including octadec-9,12-dienyl, octadec-9,12,15-trienyl, eicos-5,8,11,14-tetraenyl, farnesyl, geranyl, and geranylgeranyl, C-20 phytyl, and the like.

“Alkenylene”: The term “alkenylene” refers to a bivalent, substituted or unsubstituted, alkenyl group. A substituted alkenylene chain is a polymethylene group containing at least one double bond in which one or more hydrogen atoms are replaced with a substituent. Suitable substituents include those described herein for a substituted aliphatic group.

“Alkyl”: As used herein, the term “alkyl” means substituted or unsubstituted, saturated, straight- or branched-chain hydrocarbon radicals derived from an aliphatic moiety by removal of a single hydrogen atom. In some embodiments, the alkyl group contains 1-25 aliphatic carbon atoms. In certain embodiments, an alkyl group employed in the invention contains 10-25 carbon atoms. In certain embodiments, an alkyl group employed in the invention contains 10-20 carbon atoms. In certain embodiments, an alkyl group employed in the invention contains 15-20 carbon atoms. In certain embodiments, an alkyl group employed contains 10 carbon atoms. In certain embodiments, an alkyl group employed contains 15 carbon atoms. In certain embodiments, an alkyl group employed contains 20 carbon atoms. In certain embodiments, an alkyl group employed in the invention contains 1-3 carbon atoms. In certain embodiments, an alkyl group employed contains 1-2 carbon atoms. In certain embodiments, an alkyl group contains 1 carbon atom. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, tridecyl, tetradecyl, pentadecyl, hexadecyl, heptadecyl, octadecyl, nonadecyl, eicosyl, heneicosyl, docosyl, tricosyl, teteracosyl, pentacosyl, and the like.

“Alkylamino”: The term “alkylamino” refers to a substituted or unsubstituted group having the structure —NHR′, wherein R′ is aliphatic, as defined herein. In certain embodiments, the aliphatic group contains 1-20 aliphatic carbon atoms. In some embodiments, the aliphatic group contains 1-10 aliphatic carbon atoms. In some embodiments, the aliphatic group employed in the invention contains 1-8 aliphatic carbon atoms. In still other embodiments, the aliphatic group contains 1-6 aliphatic carbon atoms. In yet other embodiments, the aliphatic group contains 1-4 aliphatic carbon atoms. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

“Alkylene”: The term “alkylene” refers to a bivalent substituted or unsubstituted alkyl group. Unless otherwise specified, the alkylene group contains 1-25 aliphatic carbon atoms. An “alkylene chain” is a polymethylene group, i.e., —(CH₂)ₙ—, wherein n is a positive integer, preferably from 1 to 6, from 1 to 5, from 1 to 4, from 1 to 3, from 1 to 2, from 2 to 3, or 3 to 4, 4 to 5, 5 to 6. A substituted alkylene chain is a polymethylene group in which one or more methylene hydrogen atoms are replaced with a substituent. Suitable substituents include those described herein for a substituted aliphatic group.

“Alkynyl”: As used herein, the term “alkynyl” denotes a substituted or unsubstituted monovalent group derived from a straight- or branched-chain hydrocarbon moiety containing at least one carbon-carbon triple bond by removal of a single hydrogen atom. In certain embodiments, an alkynyl group employed in the invention contains 10-25 carbon atoms. In certain embodiments, an alkynyl group employed in the invention contains 10-20 carbon atoms. In certain embodiments, an alkynyl group employed contains 10 carbon atoms. In certain embodiments, an alkynyl group employed contains 15 carbon atoms. In certain embodiments, an alkynyl group employed contains 20 carbon atoms. In certain embodiments, an alkynyl group employed in the invention contains 2-3 carbon atoms. In certain embodiments, an alkynyl group employed contains 2 carbon atoms. In certain embodiments, an alkynyl group employed contains 3 carbon atoms. Representative alkynyl groups include, but are not limited to, ethynyl, 2-propynyl (propargyl), 1-propynyl, and the like.

“Alkoxy”, or “Alkylthio”: The term “alkoxy”, or “alkylthio” as used herein refers to a substituted or unsubstituted alkyl group, as previously defined, attached to the parent molecule through an oxygen atom or through a sulfur atom. In certain embodiments, the “alk” or “alkyl” portion of an “alkoxy” or “alkylthio” group contains 1-10 aliphatic carbon atoms. In yet other embodiments, the “alk” or “alkyl” portion of an “alkoxy” or “alkylthio” group employed in the present invention contains 1-8 aliphatic carbon atoms. In still other embodiments, the “alk” or “alkyl” portion of an “alkoxy” or “alkylthio” group contains 1-6 aliphatic carbon atoms. In yet other embodiments, the “alk” or “alkyl” portion of an “alkoxy” or “alkylthio” group contains 1-4 aliphatic carbon atoms. Examples of alkoxy, include but are not limited to, methoxy, ethoxy, propoxy, isopropoxy, n-butoxy, tert-butoxy, neopentoxy, and n-hexoxy. Examples of thioalkyl groups include, but are not limited to, methylthio, ethylthio, propylthio, isopropylthio, n-butylthio, and the like.

“Animal”: The term animal, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). A non-human animal may be a transgenic animal. In some embodiments, the term animal is used to refer to veterinary animals (e.g., fowl, cows, pigs, horses, etc.).

“Aralkylene” refers to a divalent group of formula —R^{a}—Ar^{a}— where R^{a} is an “alkylene” as defined herein, and Ar^{a} is an “arylene” as defined herein (i.e., an alkylene is bonded to an arylene).

“Anti-oxidant agent”: As used herein, the term “anti-oxidant agent” is an agent that inhibits oxidation or reactions promoted by oxygen or peroxides. Non-limiting examples of anti-oxidants that are usable in the context of the present invention include amines (e.g., N,N-diethylhydroxylamine, amino-guanidine), arginine pilolate, ascorbic acid (vitamin C) and its salts, ascorbyl esters of fatty acids, ascorbic acid and the like (e.g., magnesium ascorbyl phosphate, sodium ascorbyl phosphate, ascorbyl sorbate), bioflavonoids, butylated hydroxy benzoic acids and their salts, curcumin, dihydroxy fumaric acid and its salts, gallic acid and its alkyl esters (e.g., propyl gallate, uric acid and its salts and alkyl esters), glycine pidolate, grape skin/seed extracts, 6-hydroxy-2,5,7,8-tetramethylchroman-2-carboxylic acid (commercially available under the tradename Trolox®), lipoic acid, lysine, melanin, methionine, nordihydroguaiaretic acid, proline, rosemary extracts, silymarin, sorbic acid and its salts, sulfhydryl compounds (e.g., glutathione), superoxide dismutase, tea extracts, tocopherol acetate, tocopherol (vitamin E), tocopherol sorbate, and other esters of tocopherol and combinations thereof.

“Aryl” and “Heteroaryl”: In general, the terms “aryl” and “heteroaryl” refer to substituted or unsubstitued aromatic groups or moieties. In some embodiments, the terms “aryl” and “heteroaryl” may be used in the context of a different moiety name (e.g., “arylalkyl”, “aralkylene”, “aryloxy”, “heteroaryloxy” or “heteroarylalkyl”). In some embodiments, an “aryl” and/or “heteroaryl” refer to stable mono- or polycyclic, heterocyclic, polycyclic, and polyheterocyclic unsaturated moieties wherein at least one ring in the system is aromatic. In some embodiments, an “aryl” and/or “heteroaryl” ring system contains three to seven ring members. In some embodiments, an “aryl” and/or “heteroaryl” contain 3-14 carbon atoms. In certain embodiments of the present invention, “aryl” refers to a mono- or bicyclic carbocyclic ring system having one or two aromatic rings including, but not limited to, phenyl, naphthyl, tetrahydronaphthyl, indanyl, indenyl, and the like. In certain embodiments of the present invention, the term “heteroaryl”, as used herein, refers to a cyclic aromatic radical having from five to ten ring atoms of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl,oxadiazolyl, thiophenyl, furanyl, quinolinyl, isoquinolinyl, and the like. It will be appreciated that aryl and heteroaryl groups can be unsubstituted or substituted, wherein substitution includes replacement of one, two, three, or more of the hydrogen atoms thereon independently with any one or more of the moieties (e.g., “substituents”) provided herein.

“Arylene” and “Heteroarylene”: The term “arylene” refers to an unsubstituted or substituted divalent group that is carbocyclic and aromatic. In some embodiments, rings in an arylene group are fused to one another. In some embodiments rings in an arylene group are not fused, but are nonetheless connected. In some embodiments, an arylene group includes some fused rings and some connected rings. In some embodiments, an arylene group includes aromatic rings. In some embodiments, an arylene group includes non-aromatic rings. In some embodiments, an arylene group includes some aromatic rings and some non-aromatic rings. In some embodiments, the arylene group has up to 5 rings, up to 4 rings, up to 3 rings, up to 2 rings, or one aromatic ring. For example, the arylene group can be phenylene. Exemplary arylene groups include any of the “aryl” moieties listed herein with the understanding that divalency is required to arrive at a corresponding “arylene” group from an “aryl” group. Exemplary substituents of “arylene” groups include replacement of one, two, three, or more of the hydrogen atoms thereon independently with any one or more of the moieties applicable for “aryl” and “heteroaryl,” as defined herein. It will be appreciated by one skilled in the art that a carbon ring atom of an “arylene” can be replaced by one, two or three heteroatoms independently selected from S, O, and N while the remaining ring atoms are carbon, the divalent group being joined to the rest of the molecule via any two ring atoms, to form a “heteroarylene”. Exemplary “heteroarylene” groups include any of the “heteroaryl” moieties listed herein with the understanding that divalency is required to arrive at a corresponding “heteroarylene” group from a “heteroaryl” group.

“Associated with”: When two entities are “associated with” one another as described herein, they are linked by a direct or indirect covalent or non-covalent interaction. Preferably, the association is covalent. Desirable non-covalent interactions include hydrogen bonding, van der Waals interactions, hydrophobic interactions, magnetic interactions, electrostatic interactions, etc.

“Binding partner”: As used herein, the term “binding partner” refers to an agent that is non-covalently associated with an AFC compound in a complex as described herein. In some embodiments, the association between a binding partner and an AFC compound is stable in aqueous solution. In some embodiments, the association between a binding partner and an AFC compound is not stable in aqueous solution. In some embodiments, association between a binding partner and an AFC compound takes the form of a coordination complex. In some embodiments, the binding partner is a metal, a technetium isotope, a small molecule containing a basic nitrogen, a topical analgesic, an opiate, a morphinomimetic, an anti-cancer agent and/or an intraocular pressure reducing agent.

“Bivalent, branched or unbranched, saturated or unsaturated, C₂-C₆ (e.g., C₂, C₃, C₄, C₅, or C₆) hydrocarbon chain”: As used herein, the term “bivalent, branched or unbranched, saturated or unsaturated, C₂-C₆ (e.g., C₂, C₃, C₄, C₅, or C₆) hydrocarbon chain”, refers to bivalent alkylene, alkenylene, and alkynylene chains that are straight or branched as defined herein.

“Carrier”: The term “carrier” is used in accordance with its art-understood meaning, to refer to a material that is included in a pharmaceutical composition but does not abrogate the biological activity of pharmaceutically active agent(s) that are also included within the composition. Typically, carriers have very low toxicity to the animal to which such compositions are to be administered. In some embodiments, carriers are inert. In some embodiments, carriers are affirmatively beneficial (e.g., providing pharmaceutical and/or cosmetic benefits). In some embodiments, isoprenyl compounds of Formulae I (e.g., Ia, Ib, Ic, Id, Ie, If, Ig), II, III, IV, and/or V, act as acceptable carriers. In some embodiments, AFC acts as an acceptable carrier. In some embodiments, the term “carrier” when used in the pharmaceutical context (e.g., pharmaceutically acceptable carrier) means that an agent is present in a composition but does not abrogate the biological activity of another agent(s) present in a composition. In some embodiments, the term “carrier” when used in a cosmetic context (e.g., cosmetically acceptable carrier) means that an agent is present in a composition but does not but does not abrogate the biological activity and/or aesthetic effect of another agent(s) present in a composition. In some embodiments, a cosmetically acceptable carrier is used to topically administer cosmetics with which isoprenyl compounds of the present invention will remain stable and bioavailable. It will be understood that “cosmetically acceptable carriers” and “carriers” as defined herein are similar, if not often identical, in nature. In some embodiments, the term “carrier” when used in a cosmeceutical context (e.g., cosmeceutical carrier) means that an agent is present in a composition but does not abrogate the biological activity and aesthetic effect of another agent(s) present in a composition.

“Demulcent”: As used herein, the term “demulcent” is an agent used to primarily alleviate irritation, particularly mucous membranes or abraded tissues. Exemplary demulcents include acacia, agar, alginates, mucilages, benzoin, carbomer, gelatin, glycerin, gums, hydroxyethyl cellulose, hydroxypropyl cellulose, hydroxypropyl methylcellulose, hydrogels, dextrins, starches, certain sugars, and polymeric polyhydric glycols, propylene glycol, sodium alginate, tragacanth, and combinations thereof.

“Dialkylamino”: The term “dialkylamino” refers to a group having the structure —NRR′, wherein R and R′ are each an aliphatic group, as defined herein. R and R′ may be the same or different in a dialkyamino moiety. In certain embodiments, the aliphatic group contains 1-20 aliphatic carbon atoms. In some embodiments, the aliphatic group contains 1-10 aliphatic carbon atoms. In yet other embodiments, the aliphatic groups employed in the invention contain 1-8 aliphatic carbon atoms. In still other embodiments, the aliphatic group contains 1-6 aliphatic carbon atoms. In yet other embodiments, the aliphatic group contains 1-4 aliphatic carbon atoms. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of cyclic diaminoalkyl groups include, but are not limted to, aziridinyl, pyrrolidinyl, piperidinyl, morpholinyl, pyrrolyl, imidazolyl, 1,3,4-trianolyl, and tetrazolyl.

“Effective amount”: In general, the “effective amount” of an active agent (e.g., a therapeutic agent, composition, and/or formulation) refers to an amount sufficient to elicit the desired biological response. In some embodiments, a therapeutically effective amount of a substance is an amount that is sufficient, when administered to a subject suffering from or susceptible to a disease, disorder, and/or condition, to treat, diagnose, prevent, and/or delay the onset of one or more symptoms of the disease, disorder, and/or condition. As will be appreciated by those of ordinary skill in this art, and effective amount of a substance may vary depending on such factors as the desired biological endpoint, the substance to be delivered, the pharmacokinetics of the compound, the target cell or tissue, the disease being treated, the mode of administration, and the patient, etc. For example, the effective amount of a composition and/or formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. Those of ordinary skill in the art will appreciate that, commonly, a therapeutically effective amount will be administered over a series of individual doses. In some embodiments, the term “effective amount” when used in a pharmaceutical context (e.g., pharmaceutically effective amount) means that an agent is present in an amount sufficient to achieve a desired therapeutic effect. In some embodiments, the term “effective amount” when used in a cosmetic context (e.g., cosmetically effective amount) means that an agent is present in an amount sufficient to achieve an aesthetic effect. In some embodiments, the term “effective amount” when used in a cosmeceutical context (e.g., cosmeceutically effective amount) means that an agent is present in an amount sufficient to achieve a therapeutic and/or aesthetic effect. In some embodiments, a composition may be considered to contain or deliver an effective amount of a relevant agent if it contains or delivers an appropriate dose for use in a therapeutic regimen that achieves a therapeutic result when administered to an individual or when it is statistically likely to achieve a therapeutic result in when administered to a population (e.g., of individuals suffering from or susceptible to a relevant disease, disorder, or condition.

“Halo” and “Halogen”: The terms “halo” and “halogen” as used herein refer to an atom selected from fluorine, chlorine, bromine, and iodine.

“Heteroaliphatic”: The term “heteroaliphatic”, as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties (e.g., “substituents”) described herein.

“Heteroatom”: As used herein, the term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NRₓ (as in N-substituted pyrrolidinyl)).

“Heterocycle” or “Heterocyclyl”: As used herein, the terms “heterocycle”, “heterocyclyl”, “heterocyclic radical”, and “heterocyclic ring” are used interchangeably and refer to a stable 3- to 7-membered monocyclic or 7-10-membered bicyclic heterocyclic moiety that is either saturated or partially unsaturated, and having, in addition to carbon atoms, one or more, preferably one to four heteroatoms independently selected from nitrogen, oxygen, or sulfur. When used in reference to a ring atom of a heterocycle, the term “nitrogen” includes a substituted nitrogen. As an example, in a saturated or partially unsaturated ring having 1-3 heteroatoms selected from oxygen, sulfur or nitrogen, the nitrogen may be N (as in 3,4-dihydro-2H-pyrrolyl), NH (as in pyrrolidinyl), or NRₓ (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. In certain embodiments, one or more carbon atoms may be substituted with an oxo group in the heterocyclyl ring. Examples of such groups include, without limitation, an isoindolin-1,3-dione moiety. A heterocyclyl group may be mono- or bicyclic. The term “heterocyclylalkyl” refers to an alkyl group substituted by a heterocyclyl, wherein the alkyl and heterocyclyl portions independently are optionally substituted.

“Hydrocarbon”: The term “hydrocarbon”, as used herein, refers to any chemical group comprising hydrogen and carbon. In some embodiments, a hydrocarbon consists of hydrogen and carbon. A hydrocarbon may be substituted or unsubstitued. A hydrocarbon may be unsaturated, saturated, branched, unbranched, cyclic, or polycyclic. Illustrative hydrocarbons include, for example, methyl, ethyl, n-propyl, iso-propyl, cyclopropyl, allyl, vinyl, n-butyl, tert-butyl, ethynyl, cyclohexyl, methoxy, diethylamino, and the like. As would be known to one skilled in this art, all valencies must be satisfied in making any substitutions. As used herein, a “bivalent hydrocarbon” refers to alkylene, alkenylene, or alkynylene, etc.

“In combination”: As used herein, the phrase “in combination” refers to the simultaneous administration of two or more agents to a subject. It will be appreciated that two or more agents are considered to be administered “in combination” whenever a subject is simultaneously exposed to both (or more) of the agents. Each of the two or more agents may be administered according to a different schedule; it is not required that individual doses of different agents be administered at the same time, or in the same composition. Rather, so long as both (or more) agents remain in the subject's body, they are considered to be administered “in combination”.

“Independently selected”: The term “independently selected” is used herein to indicate that the R groups can be identical or different.

“Modulate”: The term “modulate” refers to change in a parameter (e.g., a change in a binding interaction or an activity, etc.). Modulation can refer to an increase or a decrease in the parameter (e.g., an increase or decrease in binding, an increase or decrease in activity, etc.).

“Non-steroidal anti-inflammatory agents”: As used herein, the term “non-steroidal anti-inflammatory agents” refers to a large group of agents that are aspirin-like in their action, including acetaminophen, Advil.RTM, Aleve.RTM, ibuprofen, naproxen sodium and Tylenol.RTM. Additional examples of non-steroidal anti-inflammatory agents that are usable in the context of the present invention include, without limitation, acetic acid derivatives (e.g., acematacin, clindanac, diclofenac, felbinac, fenclofenac, fentiazac, furofenac, indomethacin, isoxepac, ketorolac, oxepinac, sulindac, tiopinac, tolmetin, zidometacin and zomepirac), benorylate, diflunisal, disalcid, fenamates (e.g., flufenamic, meclofenamic, mefenamic, niflumic and tolfenamic acids), fendosal, oxicams (e.g., CP-14,304, isoxicam, piroxicam, sudoxicarn, and tenoxicam), propionic acid derivatives (e.g., alminoprofen, benoxaprofen, carprofen, fenbufen, fenoprofen, flurbiprofen, ibuprofen, indopropfen, ketoprofen, miroprofen, naproxen, oxaprozin, pirprofen, pranoprofen, suprofen, tiaprofenic and tioxaprofen), pyrazoles (e.g., azapropazone, feprazone, oxyphenbutazone, phenylbutazone and trimethazone), safapryn, solprin, trilisate.

“Partially unsaturated”: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

“Penetration enhancer” and “pharmaceutically acceptable penetration enhancer”: The term “penetration enhancer” and “pharmaceutically acceptable penetration enhancer” as used herein is a non-toxic agent that improves bioavailability of a topical composition. In some embodiments, a penetration enhancer is known to accelerate the delivery of a substance through the skin (e.g., disrupting the barrier function of the skin without compromising its barrier effects on microorganisms and toxins). Typically, a penetration enhancer is selected to be non-toxic to skin of the intended recipient (e.g., human). A penetration enhancer is also desirably compatible with any pharmaceutically active agent with which it is administered. Representative penetration enhancers include, for example, and without limitation, such agents as 1-substituted azacycloheptane-2-ones (e.g., 1-n-dodecylcyclazacycloheptan-2-one, available under the trademark Azone.® from Whitby Research Incorporated, Richmond, Va.), dipolar-aprotic solvents (e.g., N,N-dimethylacetamide (“DMA”), decylmethylsulfoxide (“C₁₀ MSO”), dimethyl formamide (“DMF”), dimethylsulfoxide (“DMSO”) and N-methyl-2-pyrrolidone (“NMP”)), phospholipids (e.g., allantoin, fatty acid alcohols, lecithin, alcohols including glycerols such as polyethylene glycol monolaurate (“PGML”), glycerol monolaurate (“GML”), urazole, and the like). Penetration enhancer also can be a vegetable oil, such as, but not limited to, corn oil, cottonseed oil, safflower oil, and olive oil. Additional penetration enhancers generally can be found in Remington: The Science and Practice of Pharmacy, 20^{th} ed. (Gennaro, A. R., et al., eds.) Lippincott Williams & Wilkins: Philadelphia (2000), which is incorporated herein by reference.

“Peptide”: “Peptide” as used herein has its art-understood meaning, and refers to two or more amino acids joined via an amide bond involving the carboxyl group of an amino acid and the amino group of the adjacent amino acid (i.e., a peptide bond).

“Pharmaceutically acceptable salt”: The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 66: 1-19, 1977; incorporated herein by reference. Such salts can be prepared in situ during the final isolation and purification of the compounds of the invention, or separately (e.g., by reacting the free base functionality with a suitable organic or inorganic acid). Alternatively or additionally, salts may form during formulation of a compound. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

“Pharmaceutically acceptable ester”: The term “pharmaceutically acceptable ester” refers to an ester which hydrolyzes in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic, and alkanedioic acids, in which each alkyl or alkenyl moiety advantageously has not more than 6 carbon atoms. Examples of particular esters include formates, acetates, propionates, butyrates, acrylates, and ethylsuccinates. In certain embodiments, the esters are cleaved by enzymes such as esterases.

“Pharmaceutically acceptable prodrugs”: The term “pharmaceutically acceptable prodrugs” as used herein refers to those prodrugs of active compounds which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the relevant active compound, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

“Preservative”: As used herein, the term “preservative” has its art-understood meaning and refers to an agent that protects against undesirable chemical modifications of one or more components in a composition (e.g., protection against an undesirable chemical modification of an active ingredient). Suitable preservatives for use in the compositions of the present invention include, but are not limited to, one or more alkanols, disodium EDTA, EDTA salts, EDTA fatty acid conjugates, isothioazolinone, parabens such as methylparaben and propylparaben, polypropylene glycols, sorbates, urea derivatives such as diazolindinyl urea, or combinations thereof.

“Propellant”: As used herein, the term“propellant” refers to an agent that propels the delivery of a composition in, e.g., a vaporized, aerosol nebulized, or spray form. Propellants often are used in metered-dose inhalers for the treatment of asthma and other respiratory disorders and for systemic treatments such as insulin for diabetes. Propellants also are used, for example, in nasal inhalers for treatment of allergic rhinitis, topical sprays, oral sprays, and other aerosol applications. An example of such propellants, without limitation, are the Dymel.RTM. pharmaceutical propellants manufactured by DuPont™. (Wilmington, Del.).

“Protecting Group”: One of ordinary skill in the art will appreciate that synthetic methods, such as described herein, typically utilize a variety of protecting groups. By the term “protecting group”, as used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group should be selectively removable in good yield by readily available, preferably non-toxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen, and carbon protecting groups may be utilized. Hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3 ,3a,4,5 ,6,7,7a-octahydro-7,8,8-trimethyl-4,7-methanobenzofuran-2-yl, 1-ethoxyethyl, 1- (2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(mmethoxyphenyl)phenylmethyl, tri(p-methoxyphenyl)methyl, 4-(4’-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3 ,3-tetramethylbutyl)phenoxyacetate, 2,4-bis (1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(ptoluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methane sulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide, phenyl urea, ethylurea and cyclopropyl sulfonamide. Exemplary protecting groups are detailed herein. However, it will be appreciated that the present invention is not intended to be limited to these protecting groups; rather, a variety of additional equivalent protecting groups can be readily identified using the above criteria and utilized in the method of the present invention. Additionally, a variety of protecting groups are described in Protective Groups in Organic Synthesis, Third Ed. Greene, T. W. and Wuts, P. G., Eds., John Wiley & Sons, New York: 1999, the entire contents of which are hereby incorporated by reference.

“Protective”: As used herein, the term “protective” is used to refer to an agent that isolates exposed surface of skin or other membrane from harmful or annoying stimuli. Exemplary protectives include dusting powders, adsorbents, mechanical protective agents, and plasters. Mechanical protectives are generally either collodions or plasters, and include, for example aluminum hydroxide gel, collodium, dimethicone, petrolatum gauze, absorbable gelatin film, absorbable gelatin sponge, zinc gelatin, kaolin, lanolin, anhydrous lanolin, mineral oil, mineral oil emulsion, mineral oil light, olive oil, peanut oil, petrolatum, silicones, hydrocolloids and the like. In some embodiments, a protective includes an adherent, continuous film that may be flexible or semi-rigid depending on the materials and the formulations as well as the manner in which they are applied. In some embodiments, a “protective” may be a “demulscent” as described herein.

“Small Molecule”: As used herein, the term “small molecule” refers to an organic compound, regardless or whether it is synthesized in a laboratory, found in nature, isolated or purified, etc. Typically, a small molecule is characterized in that it contains several carbon-carbon bonds, and has a molecular weight of less than 1500, although this characterization is not intended to be limiting for the purposes of the present invention.

“Solubilizing agent”: As used herein, the term “solubilizing agent” refers to a substance that enables solutes to dissolve. Representative examples of solubilizing agents that are usable in the context of the present invention include, without limitation, complex-forming solubilizers (e.g., citric acid, ethylenediamine-tetraacetate, sodium meta-phosphate, succinic acid, urea, cyclodextrin, polyvinylpyrrolidone, diethylammonium-ortho-benzoate,etc.), n-alkyl amine n-oxides, micelle-forming solubilizers (e.g., TWEEN.RTM, including TWEEN 80.RTM), organic solvents (e.g., acetone, phospholipids and cyclodextrins), polyoxamers, polyoxyethylene n-alkyl ethers, and polyoxyethylene sorbitan fatty acid ester.

“Steroidal anti-inflammatory agent”: As used herein, the term “steroidal anti-inflammatory agent”, refers to any one of numerous compounds containing a 17-carbon 4-ring system and includes the sterols, various hormones (as anabolic steroids), and glycosides. Representative examples of steroidal anti-inflammatory drugs include, without limitation, corticosteroids such as alpha-methyl dexamethasone, amcinafel, amcinafide, beclomethasone dipropionates, beclomethasone dipropionate, betamethasone and the balance of its esters, chloroprednisone, chlorprednisone acetate, clescinolone, clobetasol valerate, clocortelone, cortisone, cortodoxone, desonide, desoxycorticosterone acetate, desoxymethasone, dexamethasone, dexamethasone-phosphate, dichlorisone, dichlorisone, diflorasone diacetate, diflucortolone valerate, difluorosone diacetate, diflurosone diacetate, diflurprednate, fluadrenolone, flucetonide, fluclorolone acetonide, flucloronide, flucortine butylesters, fludrocortisone, fludrocortisone, fludrocortisone, flumethasone pivalate, flunisolide, fluocinonide, fluocortolone, fluoromethalone, fluosinolone acetonide, fluperolone, fluprednidene (fluprednylidene) acetate, fluprednisolone, fluradrenolone acetonide, fluradrenolone, flurandrenolone, halcinonide, hydrocortamate, hydrocortisone acetate, hydrocortisone butyrate, hydrocortisone valerate, hydrocortisone cyclopentylpropionate, hydrocortisone, hydroxyltriamcinolone, medrysone, meprednisone, methylprednisolone, paramethasone, prednisolone, prednisone, triamcinolone acetonide, triamcinolone, and combinations thereof.

“Substituted”: It will be appreciated that the compounds, including as described herein, may be substituted with any number of substituents or functional moieties. In general, the term “substituted” whether preceded by the term “optionally” or not, and substituents contained in formulas described herein, refers to the replacement of hydrogen radicals in a given structure with the radical of a specified substituent. When more than one position in any given structure may be substituted with more than one substituent selected from a specified group, unless otherwise indicated, the substituent may be either the same or different at every position. As used herein, the term “substituted” is contemplated to include all permissible substituents of organic compounds. In a broad aspect, permissible substituents include acyclic and cyclic, branched and unbranched, carbocyclic and heterocyclic, aromatic and nonaromatic substituents of organic moieties. Heteroatoms such as nitrogen may have hydrogen substituents and/or any permissible substituents of organic compounds described herein which satisfy the valencies of the heteroatoms. Furthermore, unless clearly so indicated, this invention is not intended to be limited in any manner by the permissible substituents of organic compounds. Combinations of substituents and variables envisioned by this invention are preferably those that result in the formation of stable compounds useful in the treatment, for example, of inflammatory diseases and/or disorders, e.g., in the modulation of a G-protein signaling cascade.

Some examples of substituents of aliphatic and other moieties of compounds provided by the present invention include, but are not limited to aliphatic; heteroaliphatic; aryl (e.g., phenyl); heteroaryl; arylalkyl; heteroarylalkyl; alkoxy; aryloxy; arylthio, heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ); —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments described herein.

“Stable”: As used herein, the term “stable” preferably refers to the state of maintaining integrity of a compound over a period of time (e.g., during manufacture and/or storage).

“Substantially free of”: As used herein, the term “substantially free of”, when used to describe a material or compound, means that the material or compound lacks a significant or detectable amount of a designated substance. In some embodiments, the designated substance is present at a level not more than about 1%, 2%, 3%, 4% or 5% (w/w or v/v) of the material or compound. In some embodiments, the designated substance is present at a level below 1%, 0.9%, 0.8%, 0.7%, 0.6%, 0.5%, 0.4%, 0.3%, 0.2%, or 0.1%.

“Surfactants”: As used herein, the term “surfactant” is a surface-active substance, such as a detergent. Suitable surfactants for use with the inventive compositions include, but are not limited to, sarcosinates, glutamates, sodium alkyl sulfates, ammonium alkyl sulfates, sodium alkyleth sulfates, ammonium alkyleth sulfates, ammonium laureth-n-sulfates, sodium laureth-n-sulfates, isothionates, glycerylether sulfonates, sulfosuccinates and combinations thereof. More particularly, an anionic surfactant is selected from the group consisting of sodium lauroyl sarcosinate, monosodium lauroyl glutamate, sodium alkyl sulfates, ammonium alkyl sulfates, sodium alkyleth sulfates, ammonium alkyleth sulfates, and combinations thereof.

“Sun screening agent”: As used herein, the term “sun screening agent” refers to an agent that, when topically applied, absorbs or reflects some of the sun's ultraviolet radiation on skin exposed to sunlight, and therefore helps protect against sunburn. In some embodiments, a sun screening agent absorbed in the skin may lead to an increase in reactive oxygen species. Representative examples of sun screening agents usable in the present invention include, without limitation, p-aminobenzoic acid and its salts and derivatives thereof (p-dimethylaminobenzoic acid; ethyl, glyceryl, and isobutyl esters;); anthranilates (i.e., o-amino-benzoates; benzyl, cyclohexenyl, linalyl, menthyl, methyl, phenyl, phenylethyl, and terpinyl esters); benzophenones (i.e., hydroxy- or methoxy-substituted benzophenones such as benzoresorcinol, butylmethoxydibenzoylmethane, 2,2′-dihydroxy-4,4′-dimethoxybenzophenone, etocrylene, 4-isopropyldibenzoylmethane, dioxybenzone, 3-4′-methylbenzylidene-boman-2-one, octabenzone, octocrylene, oxybenzene, sulisobenzone, and 2,2′,4,4′-tetrahydroxybenzophenone); (butyl carbotol) (6-propyl piperonyl) ether; cinnamic acid derivatives (alpha.-phenyl cinnamonitrile; butyl cinnamoyl pyruvate; benzyl and methyl esters); diazoles (2-acetyl-3-bromoindazole, aryl benzothiazoles, methyl naphthoxazole, and phenyl benzoxazole); dibenzylacetone; dihydroxycinnamic acid derivatives (methylaceto-umbelliferone, methylumbelliferone, umbelliferone); di-hydroxynaphthoic acid and its salts; hydrocarbons (diphenylbutadiene, and stilbene); hydroquinone; o- and p-hydroxybiphenyldisulfonates; coumarin derivatives (3-phenyl, 7-hydroxy, and 7-methyl,); naphtholsulfonates (sodium salts of 2-naphthol-3,6-disulfonic and of 2-naphthol-6,8-disulfonic acids); quinine salts (bisulfate, chloride, oleate, sulfate and tannate); quinoline derivatives (8-hydroxyquinoline salts, and 2-phenylquinoline); salicylates (amyl, benzyl, di-propylene glycol, glyceryl, menthyl, octyl, and phenyl esters); tannic acid and its derivatives (e.g., hexaethylether); trihydroxy-cinnamic acid derivatives (daphnetin, daphnin, esculetin, esculin, methylesculetin; and the glucosides); and uric and violuric acids; and combinations thereof.

“Thio”: As used herein, the term “thio” used alone or as part of a larger moiety as in “alkylthio”, “arylthio”, “heteroalkylthio”, or “heteroarylthio” refers to replacement of an oxygen. For example, “alkylthio” refers to an alkyl group, as previously defined, attached to the parent molecule through a sulfur atom. Similarly, “arylthio” refers to an aryl group, as previously defined, attached to the parent molecule through a sulfur molecule. Similarly, “heteroalkylthio” refers to a heteroalkyl group, as previously defined, attached to the parent molecule through a sulfur molecule, etc.

“Treat,” “treating” and “treatment”: As used herein, the terms “treat,” “treating” and “treatment,” contemplate an action that occurs while a patient is suffering from or susceptible to a specified disease, disorder or condition, which delays onset of and/or reduces the frequency or severity of one or more symptoms or features of the disease disorder or condition. Thus, “treat”, “treating”, and “treatment” refer to any type of treatment that imparts a benefit to a subject afflicted with a disease, disorder or condition, including improvement in the condition of the subject (e.g., in one or more symptoms), delay in the progression of the disease, disorder or condition, prevention or delay of the onset of the disease, disorder or condition, etc.

Unit dosage form: The expression “unit dosage form” as used herein refers to a physically discrete unit of a provided formulation appropriate for the subject to be treated. It will be understood, however, that the total daily usage of provided formulation will be decided by the attending physician within the scope of sound medical judgment. The specific effective dose level for any particular subject or organism may depend upon a variety of factors including the disorder being treated and the severity of the disorder; activity of specific active agent employed; specific formulation employed; age, body weight, general health, sex and diet of the subject; time of administration, and rate of excretion of the specific active agent employed; duration of the treatment; drugs and/or additional therapies used in combination or coincidental with specific complex(es) employed, and like factors well known in the medical arts. In some embodiments, a unit dosage form contains an amount of a therapeutically active agent appropriate for use in a therapeutic regimen (i.e., in a regimen that delivers a therapeutically effective amount of an agent). In some embodiments, such a unit dosage form may be considered to contain a “therapeutically effective amount” of an agent if it contains an amount appropriate for use in such a therapeutic regimen, even if a single dose would not be expected to be effective alone.

“Unsaturated”: As used herein, the term “unsaturated” means that a moiety has one or more units of unsaturation.

### DESCRIPTION OF THE DRAWINGS

FIG. 1 shows biological activity of an AFC-strontium complex, as compared with AFC alone or a strontium chloride salt.

FIG. 2 depicts certain exemplary AFC compounds.

FIG. 3 presents a line graph depicting the growth curves for Propionibacterium acnes obtained with benzoyl peroxide (BPO), AFC, AFC-Strontium complex (“AFC—Sr”), and AFC-Zinc complex (“AFC—Zn”), demonstrating antimicrobial activities.

### DETAILED DESCRIPTION OF CERTAIN EMBODIMENTS

The present invention, among other things, provides complexes comprising an AFC compound associated with a binding partner.

### 1. AFC Compounds

AFC compounds for use in accordance with the present invention include compounds with structural similarity to N-acetyl-S-farnesyl-L-cysteine (“AFC”). AFC is a signal transduction modulator that has been shown to be a competitive inhibitor of membrane-associated isoprenyl-S-cysteinyl methyltransferase and to block some neutrophil, macrophage, and platelet responses in vitro. Laboratory results indicate that AFC effectively reduces dermal inflammation in mice.

According to the present invention, AFC compounds include, for example, compounds of formula I:

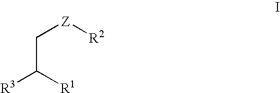

wherein:

R¹ is —C(O)X, wherein X is independently a protecting group, a halogen, R, —OR, —SR, —N(R)₂, a substituted or unsubstituted hydrazine, a substituted or unsubstituted 6-10 membered aryl ring, a substituted or unsubstituted 5-6 membered heteroaryl ring having 1-4 heteroatoms independently selected from nitrogen, oxygen, or sulfur; —NO₂; —PO₃H; —SO₃H; —CN; substituted or unsubstituted heteroaryl; or one of the following moieties:

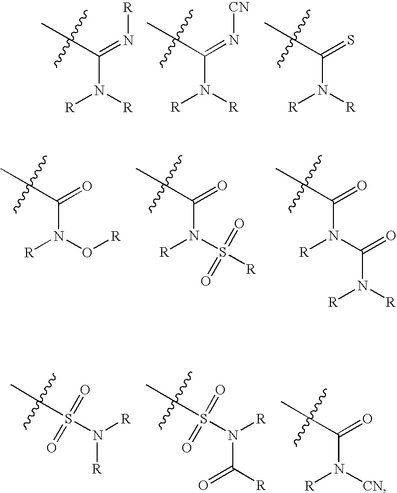

wherein each R is independently hydrogen or an optionally substituted group selected from C₁₋₆ aliphatic, C₁₋₆ heteroaliphatic, aryl, heteroaryl, or a cyclic radical;

R² is a substituted or unsubstituted, branched or unbranched C₁₀-C₂₅ aliphatic moiety;

R³ is —NH₂, a peptide, or —N(R⁴)(R⁵);

R⁴ is hydrogen or an optionally substituted group selected from C₁₋₆ aliphatic, C₁₋₆ heteroaliphatic, a cyclic radical, aryl or heteroaryl;

R⁵ is heteroaryl; —C(═N—R⁶)(R⁷), wherein R⁶ is selected from hydrogen, aliphatic, and —N(R)₂, and R⁷ is selected from hydrogen, aliphatic, aryl, cyano, and —SO₂R; or C(O)LR⁸, wherein L is a covalent bond or a bivalent, branched or unbranched, saturated or unsaturated, C₂-C₆ hydrocarbon chain wherein one or more methylene units of L is independently replaced by —O—, —S—, —NH—, —C(O)—, —C(═CH₂)—, or C₃-C₆ cycloalkylene, wherein L is optionally substituted by one or more groups selected from halogen, phenyl, an 8-10 membered bicyclic aryl ring, a 5-6 membered heteroaryl ring having 1-4 heteroatoms independently selected from nitrogen, oxygen, or sulfur, an 8-10 membered bicyclic heteroaryl ring having 1-4 heteroatoms independently selected from nitrogen, oxygen, or sulfur, a 5- to 7-membered monocyclic having 1-2 heteroatoms independently selected from nitrogen, oxygen, or sulfur or a 7-10 membered bicyclic heterocyclyl ring having 1-2 heteroatoms independently selected from nitrogen, oxygen, or sulfur; and R₈ is —R, —OR, —N(R)₂, a cyclic radical, aryl, heteroaryl, wherein each R is independently hydrogen or an optionally substituted group selected from C₁₋₆ aliphatic, C₁₋₆ heteroaliphatic, aryl, heteroaryl, or a cyclic radical; or a substituted or unsubstituted peptidic moiety; and

Z is —S—, —O—, —NH—, —Se—, —S(═O)—, —S(═N)—, —SO₂—, —Se(═O)—, or —SeO₂—.

In some embodiments, an AFC compound has a structure depicted in formula Ia:

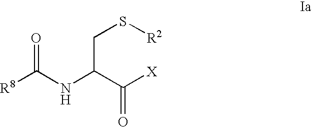

wherein R² is as defined herein;

X is —OH, halogen, methyl, —SH, —NH₂, or —N(R)₂, wherein R is hydrogen or C₁₋₃ alkyl; and

R⁸ is C₁₋₃ alkyl.

In some embodiments, an AFC compound has a structure depicted in formula Ib:

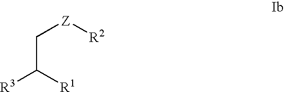

wherein

R¹ is —CO₂H, —CO₂R, —CONH₂, —NO₂, —PO₃H, —CN, or —SO₃H, where R is as defined herein;

R² is farnesyl, phytyl, geranylgeranyl, substituted farnesyl, substituted phytyl, or substituted geranylgeranyl; and

R³ is —NH₂ or a peptide.

In some embodiments, an AFC compound has a structure depicted in formula Ic:

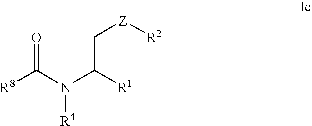

wherein R² and R⁸ are as described herein;

R¹ is substituted or unsubstituted heteroaryl, or one of the following moieties:

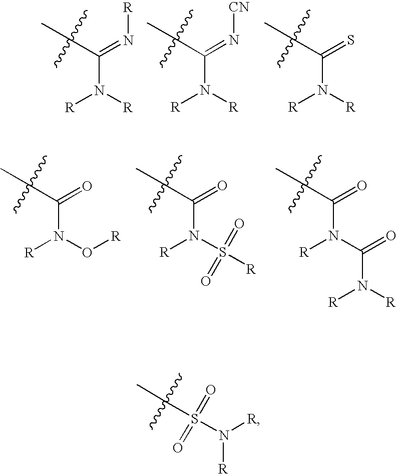

wherein R is as described herein; and

Z is —S—, —O—, —Se—, —SO—, —SO₂—, or —NH—.

In some embodiments, and AFC compound has a structure depicted in formula Id:

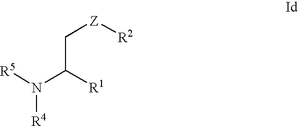

wherein R² and R⁴ are as described herein;

R¹ is substituted or unsubstituted heteroaryl, or one of the following moieties:

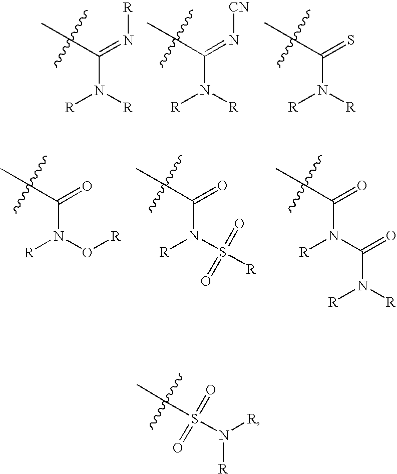

wherein R is as described herein;

R⁵ is heteroaryl or —C(═NR⁶)(R⁷), where R⁶ and R⁷ are as described herein; and

Z is —S—, —O—, —Se—, —SO—, —SO₂—, or —NH—.

In some embodiments, an AFC compound has a structure depicted in formula Ie:

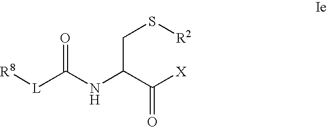

wherein R² is as described herein;

X is R, —OR, a hydrogen, aryloxy, amino, alkylamino, dialkylamino, heteroaryloxy, hydrazine, a 6-10 membered aryl ring, a 5-6 membered heteroaryl ring having 1-4 heteroatoms independently selected from nitrogen, oxygen, or sulfur, wherein each R is independently hydrogen or an optionally substituted group selected from C₁₋₆ aliphatic or C₁₋₆ heteroaliphatic;

L is a bivalent, branched or unbranched, saturated or unsaturated, C₂-C₆ hydrocarbon chain wherein one or more methylene units of L is independently replaced by —O—, —S—, —NH—, —C(O)—, —C(═CH₂)—, or C₃-C₆ cycloalkylene, wherein L is optionally substituted by one or more groups selected from halogen, phenyl, an 8-10 membered bicyclic aryl ring, a 5-6 membered heteroaryl ring having 1-4 heteroatoms independently selected from nitrogen, oxygen, or sulfur, an 8-10 membered bicyclic heteroaryl ring having 1-4 heteroatoms independently selected from nitrogen, oxygen, or sulfur, a 5- to 7-membered monocyclic having 1-2 heteroatoms independently selected from nitrogen, oxygen, or sulfur or a 7-10 membered bicyclic heterocyclyl ring having 1-2 heteroatoms independently selected from nitrogen, oxygen, or sulfur; and

R⁸ is hydrogen, —OH or —OR, wherein each R is independently hydrogen or an optionally substituted group selected from C₁₋₆ aliphatic or C₁₋₆ heteroaliphatic.

In some embodiments, an AFC compound has a structure depicted in formula If:

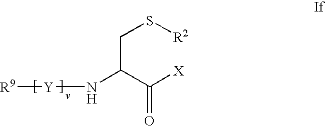

wherein

Y is a natural or unnatural amino acid;

v is an integer between 1 and 100, inclusive; and

R⁹ is hydrogen, a protecting group, or an optionally substituted group selected from C₁₋₆ aliphatic, C₁₋₆heteroaliphatic, aryl or heteroaryl.

In some embodiments, an AFC compound has a structure depicted in formula Ig:

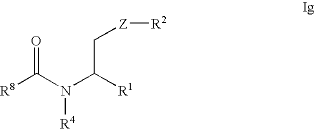

or a pharmaceutically acceptable salt, enantiomer, diastereomer, or double bond isomer thereof, wherein:

Z is —S—, —O—, —Se—, —S(O)—, —SO₂—, or —NH—;

R¹ is a heteroaryl group, or a moiety selected from

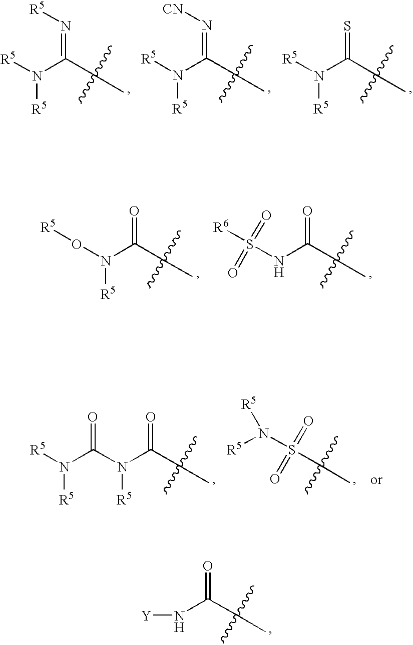

wherein at least one R⁵ group is H;

R⁵ is independently selected from H, alkyl, aryl, alkenyl, or alkynyl, wherein R⁵ is optionally substituted with one or two R⁷ groups;

R⁶ is H, alkyl, aryl, alkenyl, alkynyl, or a cyclic radical, where R⁶ is optionally substituted with one or two R⁷ groups;

Y is selected from H, —NH₂, —OH, —NH-phenyl, —NHC(O)CH₃, —NHCH₃, or —(C₁-C₈)alkyl;

R² is an aliphatic group substituted with one or more R⁷ groups;

R⁸ is alkoxy, aminoalkyl, alkyl, aryl, alkenyl, alkynyl, or a cyclic radical, where R⁸ is optionally substituted with one or two R⁷ groups;

R⁴ is H, alkyl, aryl, alkenyl, alkynyl, or a cyclic radical, where R⁴ is optionally substituted with one or two R⁷ groups; and

R⁷ is —NHC (═O)(C₁-C₈)alkyl, —(C₁-C₈)alkyl, —(C₁-C₈)alkenyl, —(C₁-C₈)alkynyl, phenyl, —(C₂-C₅)heteroaryl, —(C₁-C₆)heterocycloalkyl, —(C₃-C₇)cycloalkyl, —O—(C₁-C₈), —O—(C₁-C₈)alkenyl, —O—(C₁-C₈)alkynyl, —O-phenyl, —CN, —OH, oxo, halo, —C(═O)OH, —COhalo, —OC (═O)halo, —CF₃, N₃, NO₂, —NH₂, —NH((C₁-C₈)alkyl), —N((C₁-C₈)alkyl)₂, —NH(phenyl), —N(phenyl)₂, —C(═O)NH₂, —C(═O)NH((C₁-C₈)alkyl), —C(═O)N((C₁-C₈)alkyl)₂, —C(═O)NH(phenyl), —C(═O)N(phenyl)₂, —OC(═O)NH₂, —NHOH, —NOH((C₁-C₈)alkyl), —NOH(phenyl), —OC(═O)NH((C₁-C₈)alkyl), —OC(═O)N((C₁-C₈)alkyl)₂, —OC(═O)NH(phenyl), —OC(═O)N(phenyl)₂, —CHO, —CO((C₁-C₈)alkyl), —CO(phenyl), —C(═O)O((C₁-C₈)alkyl), —C(═O)O(phenyl), —OC(═O)((C₁-C₈)alkyl), —OC(═O)(phenyl), —OC(═O)O((C₁-C₈)alkyl), —OC(═O)O(phenyl), —S—(C₁-C₈)alkyl, —S—(C₁-C₈)alkenyl, —S—(C₁-C₈)alkynyl, and —S-phenyl, —NHS(O)₂-phenyl, —NHS(O)₂-alkyl, —NHS(O)₂—(C₁-C₈)alkenyl, —NHS(O)₂-(C₁-C₈)alkynyl, —SC(O)-phenyl, —SC(O)-alkyl, —SC(O)—(C₁-C₈)alkenyl, —SC(O)—(C₁-C₈ alkynyl), —O—S(═O)₂—(C₁-C₈)alkyl, —O—S(═O)₂—(C₁-C₈)alkenyl, —O—S(═O)₂—(C₁-C₈)alkynyl, —O—S(═O)₂-phenyl, —(CH₂)ₙNH₂, —(CH₂)ₙ—NH((C₁-C₈)alkyl), —(CH₂)ₙN((C₁-C₈)alkyl)₂, —(CH₂)ₙNH(phenyl), or —(CH₂)ₙN(phenyl)₂, wherein n is 1 to 8.

In some embodiments of any of the foregoing structures I and Ia-Ig, R¹ is an optionally substituted heteroaryl moiety of one of the formulae:

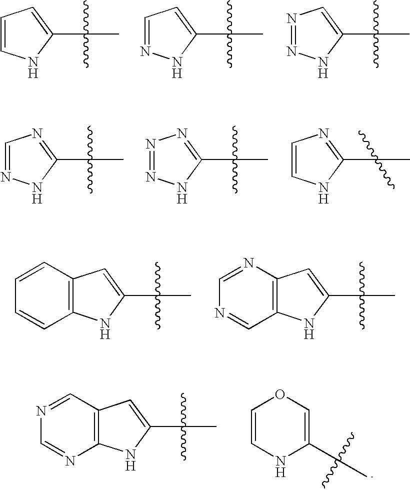

In some embodiments, R¹ is —CO₂H.

In some embodiments of any of the foregoing structures I and Ia-Ig, R² is a farnesyl group.

In some embodiments of any of the foregoing structures I and Ia-Ig, R³ is —NHCOCH₃.

In some embodiments of any of the foregoing structures I and Ia-Ig, Z is —S—.

In some embodiments of any of the foregoing structures I and Ia-Ig, X is —OH.

In some embodiments, an AFC compound has a structure depicted in formula II:

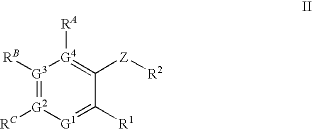

wherein each of G¹, G², G³, and G⁴ is N or CR^{D};

Z is S, O, Se, SO, SO₂, or NH;

R¹ is —C(O)X, wherein X is independently a protecting group, a halogen, R, —OR, —SR, —N(R)₂, a substituted or unsubstituted hydrazine, a substituted or unsubstituted 6-10 membered aryl ring, a substituted or unsubstituted 5-6 membered heteroaryl ring having 1-4 heteroatoms independently selected from nitrogen, oxygen, or sulfur; —NO₂; —PO₃H; —SO₃H; —CN; substituted or unsubstituted heteroaryl; or one of the following moieties:

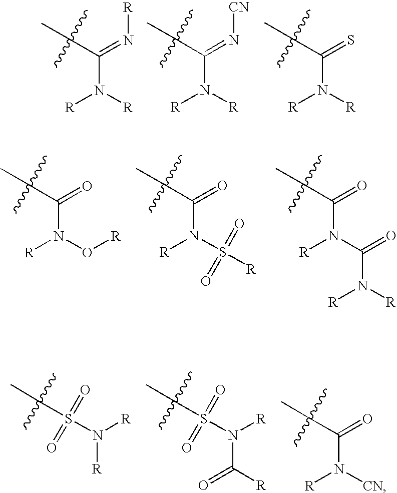

wherein each R is independently hydrogen or an optionally substituted group selected from C₁₋₆ aliphatic, C₁₋₆heteroaliphatic, aryl, heteroaryl, or a cyclic radical;

R² is an optionally substituted aliphatic group;

R^{A}, R^{B}, R^{C}, and R^{D} are independently H, —NO₂, —OR¹⁰, halogen, alkylN(R¹⁰)₂, —N(R¹⁰)₂, —C(═O)R¹⁰, —C(═O)R¹⁰, —S(R¹⁰), azido, —S—C≡N, alkyl, aryl, alkenyl, alkynyl, or a cyclic radical, wherein R^{A}, R^{B}, R^{C}, and R^{D} are further optionally substituted;

R¹⁰ is H, alkyl, aryl, alkenyl, alkynyl, or a cyclic radical, wherein R¹⁰ is further optionally substituted.

In some embodiments, at least one of G¹, G², G³, and G⁴ is N; in some embodiments, at least two of G¹, G², G³, and G⁴ are N; in some embodiments, at least three of G¹, G², G³, and G⁴ are N; in some embodiments, at least four of G¹, G², G³, and G⁴ are N. In some embodiments, G¹ is N. In some embodiments, G¹ is N and at least one of G², G³, and G⁴ is N.

In some embodiments, an AFC compound has a structure depicted in formula

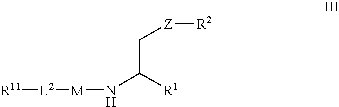

wherein:

L² is a bivalent, branched or unbranched, saturated or unsaturated, C₂-C₆ hydrocarbon chain wherein one or more methylene units of L is independently replaced by —O—, —S—, —NH—, —C(O)—, —CF₂—, —C(═CH₂)—, —CH═CH—, or an optionally substituted arylene, heteroarylene, C₃-C₆ cycloalkylene, C₃-C₆ heterocycloalkylene, or an 8-10-membered bicyclic heterocyclic moiety,

and wherein L² is optionally substituted by one or more groups selected from halogen, C₁-C₆ alkyl, phenyl, biphenyl, -benzyl, —CH₂-phenol, —CH(phenyl)₂, —OH, —NH₂, —NHC(O)CH₃, —NHC(O)NHCH₂CH₃, —C(O)NH₂, —C(O)NHCH₂CH₃, —CH₂C(O)OCH₂phenyl, —(CH₂)₂SCH₃, —(CH₂)₂C(O)NH₂, —(CH₂)₂C(O)OH, an 8-10 membered bicyclic aryl ring, a 5-6 membered heteroaryl ring having 1-4 heteroatoms independently selected from nitrogen, oxygen, or sulfur, an 8-10 membered bicyclic heteroaryl ring having 1-4 heteroatoms independently selected from nitrogen, oxygen, or sulfur, a 5- to 7-membered monocyclic having 1-2 heteroatoms independently selected from nitrogen, oxygen, or sulfur or a 7-10 membered bicyclic heterocyclyl ring having 1-2 heteroatoms independently selected from nitrogen, oxygen, or sulfur;

M is —C(O)—, —C(S), or —SO₂—;

R¹¹ is hydrogen, F, CF₃, C₁-C₄ alkyl, —OH, —C(O)CH₃, —NH(OR¹²), —N(R¹²)₂, —NHN(R¹²)₂, —SO₂R¹², —NH-phenyl, —SO₂-phenyl, -phenyl-NO₂, or —OR¹², wherein each R¹² is independently hydrogen or an optionally substituted group selected from C₁₋₆ aliphatic or C₁-₆ heteroaliphatic;

R¹ is —C(O)X, wherein X is independently R¹², —C(O)NHNH₂, —OR¹², a hydrogen, aryloxy, amino, alkylamino, dialkylamino, heteroaryloxy, hydrazine, a 6-10 membered aryl ring, a 5-6 membered heteroaryl ring having 1-4 heteroatoms independently selected from nitrogen, oxygen, or sulfur; and

R² is a substituted or unsubstituted, branched or unbranched C₁₀-C₂₅ aliphatic moiety;

Z is —O—, —N—, —S—, —Se—, —S(O)—, —S(═N)—, —SO₂—, —Se(O)—, or —Se(O)₂—.

In some embodiments of formula III, R¹ is an optionally substituted heteroaryl moiety of one of the formulae:

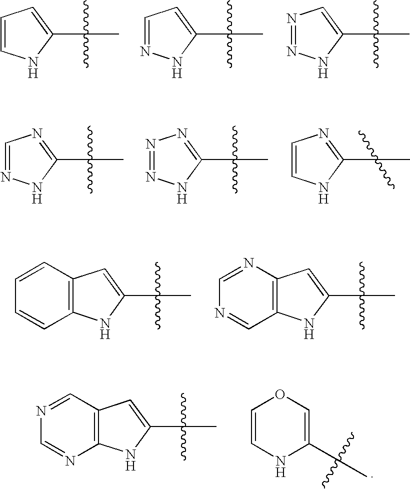

In some embodiments, R¹ is —CO₂H.

In some embodiments of formula III, R² is a farnesyl group.

In some embodiments of formula III, Z is —S—.

In some embodiments, an AFC compound has a structure depicted in formula IV:

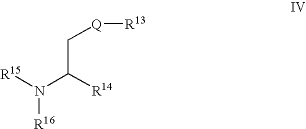

wherein:

R¹⁴ is an optionally substituted heteroaryl group or:

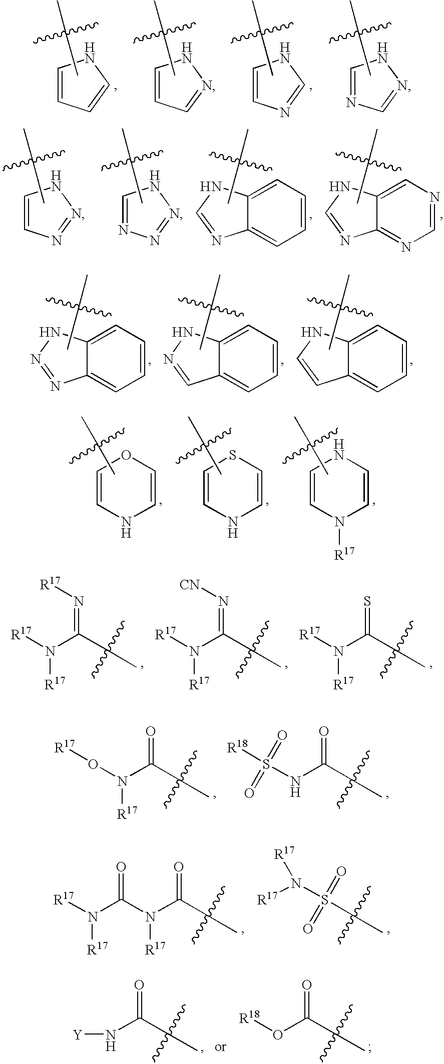

R¹³ is an aliphatic group substituted with one or more R¹⁹ groups;

R¹⁵ is an optionally substituted heteroaryl group, or a group selected from:

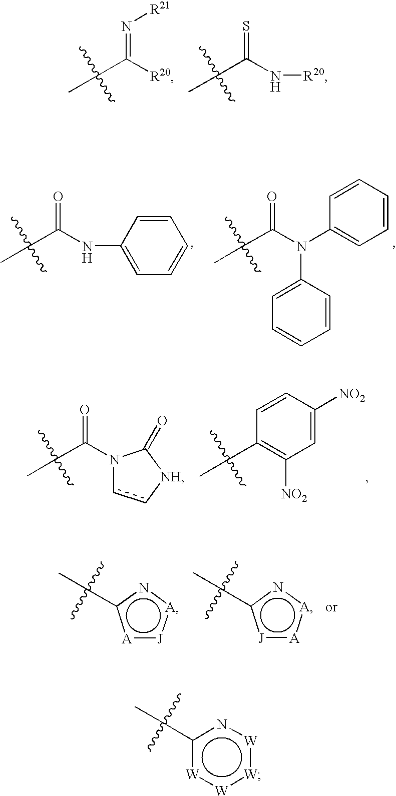

Y is selected from H, —NH₂, —OH, —NH-phenyl, —NHC(O)CH₃, —NHCH₃, or —(C₁-C₈)alkyl;

W is independently —C(R²²)— or N;

R²² is halo, hydrogen, CF₃, N(R¹⁷)₂, oxo, alkyl, alkenyl, alkynyl or aryl;

J is —O—, S, —N—, —N(R¹⁷)—, —C(R²³)— or —C(R¹⁸)—;

A is independently —C(R²³)—, —N— or —O—;

R²³ is hydrogen, F, CH₃, CF₃, OH, —NH₂, —NHNH₂, alkyl, alkenyl, alkynyl or aryl;

R¹⁶ is H, alkyl, aryl, alkenyl, alkynyl, or a cyclic radical, wherein R¹⁶ is optionally substituted with one or two R¹⁹ groups;

R¹⁷ is independently H, alkyl, aryl, alkenyl, or alkynyl, or —C(═O)O-t-butyl wherein R¹⁷ is optionally substituted with one or two R¹⁹ groups;

R¹⁸ is H, alkyl, aryl, alkenyl, alkynyl, or a cyclic radical, wherein R¹⁸ is optionally substituted with one or two R¹⁹ groups;

R¹⁹ is —NHC(═O)(C₁-C₈)alkyl, —(C₁-C₈)alkyl, —(C₁-C₈)alkenyl, —(C₁-C₈)alkynyl, phenyl, —(C₂-C₅)heteroaryl, —(C₁-C₆)heterocycloalkyl, —(C₃-C₇)cycloalkyl, —O—(C₁-C₈)alkyl, —O—(C₁-C₈)alkenyl, —O—(C₁-C₈)alkynyl, —O-phenyl, —CN, —OH, oxo, halo, —C(═O)OH, —COhalo, —OC(═O)halo, —CF₃, N₃, NO₂, —NH₂, —NH((C₁-C₈)alkyl), —N((C₁-C₈)alkyl)₂, —NH(Phenyl), —N(phenyl)₂, —C(═O)NH₂, —C(═O)NH((C₁-C₈)alkyl), —C(═O)N((C₁-C₈)alkyl)₂, —C(═O)NH(phenyl), —C(═O)N(phenyl)₂, —OC(═O)NH₂, —NHOH, —NOH((C₁-C₈)alkyl), —NOH(phenyl), —OC(═O)NH((C₁-C₈)alkyl), —OC(═O)N((C₁-C₈)alkyl)₂, —OC(═O)NH(phenyl), —OC(═O)N(phenyl)₂, —CHO, —CO((C₁-C₈)alkyl), —CO(phenyl), —C(═O)O((C₁-C₈)alkyl), —C(═O)O(phenyl), —OC(═O)((C₁-C₈)alkyl), —OC(═O)(phenyl), —OC(═O)O((C₁-C₈)alkyl —OC(═O)O(phenyl), —S—(C₁-C₈)alkyl, —S—(C₁-C₈)alkenyl, —S—(C₁-C₈)alkynyl, and —S-phenyl, —NHS(O)₂-phenyl, —NHS(O)₂-alkyl, —NHS(O)₂—(C₁-C₈)alkenyl, —NHS(O)₂—(C₁-C₈)alkynyl, —NHS(O)₂, —SC(O)-phenyl, —SC(O)-alkyl, —SC(O)—(C₁-C₈)alkenyl, —SC(O)—(C₁-C₈ alkynyl), —O—S(═O)₂—(C₁-C₈)alkyl, —O—S(═O)₂—(C₁-C₈)alkenyl, —O—S(═O)₂-phenyl, —(CH₂)ₙNH₂, —(CH₂)ₙ—NH((C₁-C₈)alkyl), —(CH₂)ₙN((C₁-C₈)alkyl)₂, —(CH₂)ₙNH(phenyl), or —(CH₂)ₙN(phenyl)₂, wherein n is 1 to 8;

R²⁰ is H, alkyl, alkenyl, alkynyl, aryl, —N(R¹⁷)₂;

R²¹ is H, alkyl, alkenyl, alkynyl, aryl, —CN, —S(═O)₂—R¹⁸ or —C(═O)O-t-butyl; and

Q is —S—, —O—, —Se—, —S(O)—, —SO₂—, or —NH—;

each of the dashed lines independently represents the presence or absence of a double bond.

In some embodiments of formula IV, R¹ is an optionally substituted heteroaryl moiety of one of the formulae:

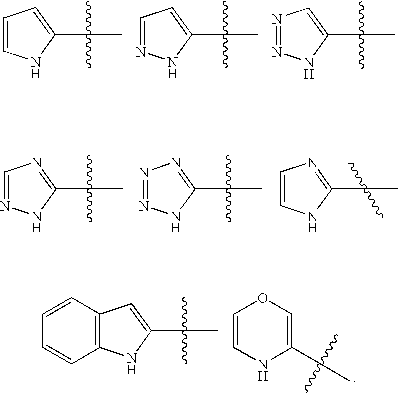

In some embodiments of formula IV, R¹³ is a farnesyl group.

In some embodiments of formula IV, Q is —S—.

In some embodiments, an AFC compound has a structure depicted in formula V:

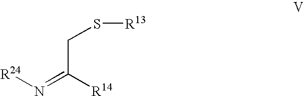

wherein:

R¹⁴ is an optionally substituted heteroaryl group or:

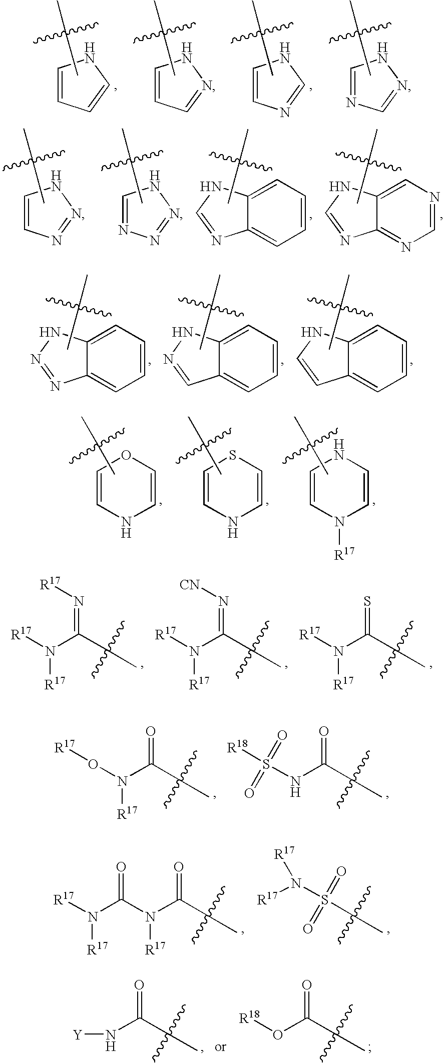

Y is selected from H, —NH₂, —OH, —NH-phenyl, —NHC(O)CH₃, —NHCH₃, or —(C₁-C₈)alkyl;

R¹³ is an aliphatic group substituted with one or more R¹⁹ groups;

R²⁴ is independently H,

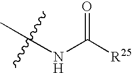

### or —NH—S(O)₂R²⁵;

R²⁵ is independently H, (C₁-C₄)alkyl or aryl;

R¹⁷ is independently H, alkyl, aryl, alkenyl, or alkynyl, wherein R¹⁷ is optionally substituted with one or two R¹⁹ groups;

R¹⁸ is H, alkyl, aryl, alkenyl, alkynyl, or a cyclic radical, wherein R¹⁸ is optionally substituted with one or two R¹⁹ groups;

R¹⁹ is —NHC(═O)(C₁-C₈)alkyl, —(C₁-C₈)alkyl, —(C₁-C₈)alkenyl, —(C₁-C₈)alkynyl, phenyl, —(C₂-C₅)heteroaryl, —(C₁-C₆)heterocycloalkyl, —(C₃-C₇)cycloalkyl, —O—(C₁-C₈)alkyl, —O—(C₁-C₈)alkenyl, —O—(C₁-C₈)alkynyl, —O-phenyl, —CN, —OH, oxo, halo, —C(═O)OH, —COhalo, —OC(═O)halo, —CF₃, N₃, NO₂, —NH₂, —NH((C₁-C₈)alkyl), —N((C₁-C₈)alkyl)₂, —NH(phenyl), —N(phenyl)₂, —C(═O)NH₂, —C(═O)NH((C₁-C₈)alkyl), —C(═O)N((C₁-C₈)alkyl)₂, —C(═O)NH(phenyl), —C(═O)N(phenyl)₂, —OC(═O)NH₂, —NHOH, —NOH((C₁-C₈)alkyl), —NOH(phenyl), —OC(═O)NH((C₁-C₈)alkyl), —OC(═O)N((C₁-C₈)alkyl)₂, —OC(═O)NH(phenyl), —OC(═O)N(phenyl)₂, —CHO, —CO((C₁-C₈)alkyl), -CO(phenyl), —C(═O)O((C₁-C₈)alkyl), —C(═O)O(phenyl), —OC(═O)((C₁-C₈)alkyl), —OC(═O)(phenyl), —OC(═O)O((C₁-C₈)alkyl), —OC(═O)O(phenyl), —S—(C₁-C₈)alkyl, —S—(C₁-C₈)alkenyl, —S—(C₁-C₈)alkynyl, and —S-phenyl, —NHS(O)₂-phenyl, —NHS(O)₂-alkyl, —NHS(O)₂—(C₁-C₈)alkenyl, —NHS(O)₂—(C₁-C₈)alkynyl, —NHS(O)₂, —SC(O)-phenyl, —SC(O)-alkyl, —SC(O)—(C₁-C₈)alkenyl, —SC(O)—(C₁-C₈ alkynyl), —O—S(═O)₂—(C₁-C₈)alkyl, —O—S(═O)₂—(C₁-C₈)alkenyl, —O—S(═O)₂—(C₁-C₈)alkynyl, —O—S(═O)₂-phenyl, —(CH₂)ₙNH₂, —(CH₂)ₙ—NH((C₁-C₈)alkyl), —(CH₂)ₙN((C₁-C₈)alkyl)₂, —(CH₂)ₙNH(phenyl), or —(CH₂)ₙN(phenyl)₂, wherein n is 1 to 8.

In some embodiments of formula V, R¹ is an optionally substituted heteroaryl moiety of one of the formulae:

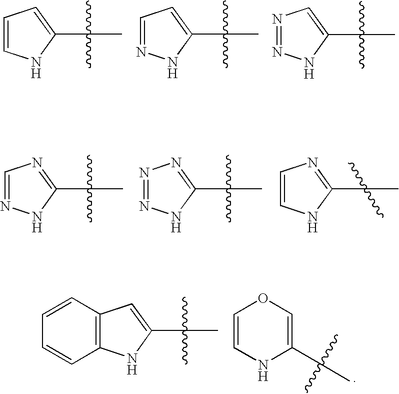

In some embodiments of formula V, R¹³ is a farnesyl group.

AFC, and many AFC compounds are characterized by an ability to reduce methylation of a protein having a carboxyl-terminal —CAAX motif, wherein C=cysteine, A=any aliphatic amino acid, and X=any amino acid. (See Rando, U.S. Pat. No. 5,202,456). The methylation reaction which is inhibited is part of a series of post-translational modifications involving the —CAAX motif. These modifications include polyisoprenylation of the cysteine of the —CAAX motif (on the sulfur), proteolysis of the carboxyl-terminal three amino acids (-AAX) and methylation of the carboxyl group of cysteine.

In certain embodiments, an AFC compound is disclosed in U.S. Pat. No. 5,043,268; U.S. Pat. No. 5,202,456; U.S. Provisional Application 61/007,234, filed Dec. 10, 2007; U.S. Provisional Application 61/065,939, filed Feb. 14, 2008; U.S. Provisional Application 61/113,498, filed Nov. 11, 2008; U.S. Publication 2009/0170917, published on Jul. 2, 2009; U.S. Provisional Application 61/066,075, filed on Feb. 15, 2008; U.S. application Ser. No. 12/616,781, filed Nov. 12, 2009; or PCT Publication WO2009/102997, published on Aug. 20, 2009; each of which is incorporated herein by reference.

### 2. Binding Partners

As mentioned above, a “binding partner” is an agent that is associated with an AFC compound in a complex as described herein.

In some embodiments, the binding partner can be a neutral, charged (i.e., in the form of an ion) or uncharged entity.

In certain embodiments, a binding partner is in the form of an ion.

In certain embodiments, a binding partner comprises a metal. In certain embodiments, the metal is selected from the group consisting of bismuth, cadmium, calcium, chromium, cobalt, copper, gold, iron, manganese, molybdenum, platinum scandium, silver, sodium, strontium, technetium, tin, titanium, vanadium, yttrium, zinc, and combinations thereof. In certain embodiments, the metal is a transition metal. In certain embodiments, the metal is strontium. In certain embodiments, the metal is calcium. In certain embodiments, the metal is sodium. In certain embodiments, the metal is zinc. In certain embodiments, the metal is titanium. In certain embodiments, the metal is silver.

In certain embodiments, a binding partner comprises a small molecule containing a basic nitrogen. In certain embodiments, the small molecule is glucosamine. In certain embodiments, the small molecule is nicotinamide. In certain embodiments, the small molecule is an NSAID. Exemplary NSAIDS include ampyrone, azapropazone, phenazone, piroxicam, droxicam, lornoxicam, tenoxicam and etoricoxib.

In certain embodiments, a binding partner comprises a topical analgesic. In certain embodiments, the topical analgesic may is selected from benzocaine, butamben, dibucaine, lidocaine, oxybuprocaine, pramoxine, proparacaine, proxymetacaine and tetracaine.

In certain embodiments, a binding partner comprises an opiate. Exemplary opiates include codeine, diamorphine, hydrocodone, morphine, naloxone, naltrexone, oxycodone, pethidine, etc.

In certain embodiments, a binding partner comprises a morphinomimetic. Exemplary morphinomimetics include meperidine and other phenylpiperidine derivatives (e.g., alfentanil, fentanyl, remifentanil, sufentanil, etc.).

In certain embodiments, a binding partner comprises an anti-cancer agent. Exemplary anti-cancer agents include camptothecin, irinotecan, lamellarin D, mitomycin, nitrogen mustards, temozolomide, topotecan, vinblastine, vincristine, vindesine, vinorelbine, etc.

In certain embodiments, a binding partner comprises an intraocular pressure reducing agent. Exemplary intraocular pressure reducing agents include brimonidine, timolol, etc.

In certain embodiments, a binding partner comprises a skin whitening agent. Exemplary skin whitening agents include hydroquinone, metronidazole, niacineamide (nicotineamide), etc. In certain embodiments, the skin whitening agent is niacineamide (nicotineamide).

In certain embodiments, a binding partner has anti-inflammatory activity. In certain embodiments, the AFC compound has anti-inflammatory activity. In certain embodiments, the AFC compound does not have anti-inflammatory activity. In certain embodiments, a provided complex shows anti-inflammatory activity that is comparable to or greater than that of the uncomplexed AFC compound. In certain embodiments, a provided complex shows anti-inflammatory activity that is comparable or greater than of the uncomplexed binding partner. In certain embodiments, a provided complex shows anti-inflammatory activity that is greater than the sum of the activities of the uncomplexed AFC compound and binding partner.

In certain embodiments, a binding partner has anti-microbial activity. In certain embodiments, the AFC compound has anti-microbial activity. In certain embodiments, the AFC compound does not have anti-microbial activity. In certain embodiments, a provided complex shows anti-microbial activity that is comparable to or greater than that of the uncomplexed AFC compound. In certain embodiments, a provided complex shows anti-microbial activity that is comparable or greater than of the uncomplexed binding partner. In certain embodiments, a provided complex shows anti-microbial activity that is greater than the sum of the activities of the uncomplexed AFC compound and binding partner.

In certain embodiments, a binding partner has sun-blocking activity. In certain embodiments, the AFC compound has sun-blocking activity. In certain embodiments, the AFC compound does not have sun-blocking activity. In certain embodiments, a provided complex shows sun-blocking activity that is comparable to or greater than that of the uncomplexed AFC compound. In certain embodiments, a provided complex shows sun-blocking activity that is comparable or greater than that of the uncomplexed binding partner.

In certain embodiments, a provided complex shows sun-blocking activity that is greater than the sum of the activities of the uncomplexed AFC compound and binding partner.

### 3. Complexes

The present invention encompasses the finding that a complex comprising an AFC binding partner and a binding partner that is non-covalently associated thereto has desirable attributes, including for example, abilities to inhibit or reduce sensory irritation, MPO, erythema, edema and or vesiculation. In some embodiments, inventive complexes have inhibitory activity similar to or greater than that observed with the corresponding uncomplexed AFC compound; in some embodiments, inventive complexes have inhibitory activity similar to or greater than uncomplexed AFC.

In some embodiments, a provided complex has salt-like character. In other embodiments, a provided complex does not have salt-like character. In certain embodiments, a provided complex is a coordination complex. In some embodiments, a provided complex has d orbital involvement in the non-covalent interaction between compound and binding partner. In some embodiments, a provided complex exists as a solvent separated ion pair. In other embodiments, a provided complex does not exist as a solvent separated ion pair. In some embodiments, a provided binding partner is readily exchangeable with another binding partner. In other embodiments, a provided binding partner is not readily exchangeable with another binding partner. In some embodiments, a provided binding partner is monodentate or monovalent. In other embodiments, a provided binding partner is not monodentate or monovalent.

In certain embodiments, a provided complex is characterized by ¹H NMR. In certain embodiments, a provided complex is characterized by one or more changes in chemical shift relative to the corresponding uncomplexed compound of formula I, II, III, IV, or V. In certain embodiments, a provided complex is characterized by line broadening relative to the corresponding uncomplexed compound of formula I, II, III, IV, or V. In certain embodiments, a provided complex is characterized by both line broadening relative to the corresponding uncomplexed compound of formula I, II, III, IV, or V, and by one or more changes in chemical shift relative to the corresponding uncomplexed compound of formula I, II, III, IV, or V.

In some embodiments, the binding partner and compound of formula I, II, III, IV, or V are present in a ratio within the range of about 0.5:4.5 to 2:1. In some embodiments, the binding partner and compound of formula I, II, III, IV, or V are present in a ratio of about 1:4. In some embodiments, the binding partner and compound of formula I, II, III, IV, or V are present in a ratio of about 1:1. In some embodiments, the binding partner and compound of formula I, II, III, IV, or V are present in a ratio of about 2:1. In some embodiments, the binding partner and compound of formula I, II, III, IV, or V are present in a ratio of about 1:2. In some embodiments, the binding partner and compound of formula I, II, III, IV, or V are present in a ratio of about 0.5:4.5. In some embodiments, the binding partner and compound of formula I, II, III, IV, or V are present in a ratio of about 1:3. In certain embodiments, the binding partner and compound of formula I, II, III, IV, or V are not present in a ratio of about 1:1. In other embodiments, the binding partner and compound of formula I, II, III, IV, or V are not present in a ratio of about 2:1.

In some particular embodiments, when the binding partner is a metal, the binding partner and compound of formula I, II, III, IV, or V are present in a ratio of about 1:4. In certain such embodiments, the binding partner comprises strontium. In some particular embodiments, when the binding partner is a metal, the binding partner and compound of formula I, II, III, IV, or V are present in a ratio of about 2:1. In certain such embodiments, the binding partner comprises sodium.

In some particular embodiments, when the binding partner is a small molecule containing a basic nitrogen, the binding partner and compound of formula I, II, III, IV, or V are present in a ratio of about 1:1. In certain such embodiments, the binding partner comprises glucosamine. In certain other such embodiments, the binding partner comprises nicotinamide. In certain embodiments, when the ratio of binding partner:compound is 1:1, the binding partner comprises sodium.

In certain embodiments, the present invention provides complexes having the structural formula:

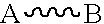

wherein:

[symbol] represents a non-covalent association;

A represents a compound of formula I, II, III, IV, or V; and

B represents a binding partner.

In certain embodiments, the present invention provides complexes having the structural formula:

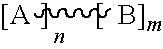

wherein:

[symbol] represents a non-covalent association;

A represents a compound of formula I, II, III, IV, or V;

B represents a binding partner;

n represents an number in the range of 1 to 9, inclusive; and

m represents an number in the range of 1 to 2, inclusive.

In certain embodiments, n is 4 and m is 1. In other embodiments, n is 1 and m is 1. In some embodiments, n is 2 and m is 1. In other embodiments, n is 1 and m is 2.

In certain embodiments, wherein R¹ is an acetyl group, R² is a carboxyl group and R³ is a farnesyl group, the present invention provides a complex comprising a compound of formula (Ih), i.e., N-acetyl-S-farnesyl-L-cysteine (AFC):

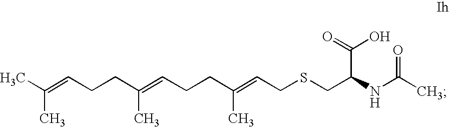

and a binding partner.

In certain embodiments, wherein R¹ is an succinyl group, R² is a carboxyl group and R³ is a farnesyl group, the present invention provides a complex comprising a compound of formula (Ii), i.e., N-succinyl-S-farnesyl-L-cysteine (SFC):

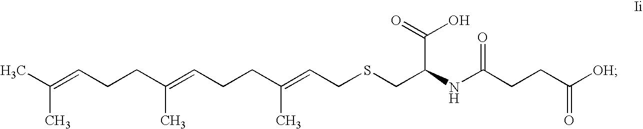

and a binding partner.

In certain embodiments, wherein R¹ is an malonyl group, R² is a carboxyl group and R³ is a farnesyl group, the present invention provides a complex comprising a compound of formula (Ij), i.e., N-malonyl-S-farnesyl-L-cysteine (MFC):

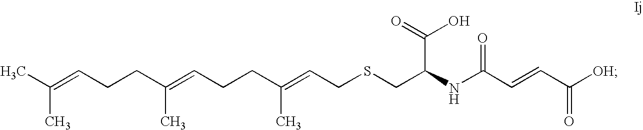

and a binding partner.

In certain embodiments, wherein the compound is of formula Ih and the binding partner is calcium, the complex is of formula:

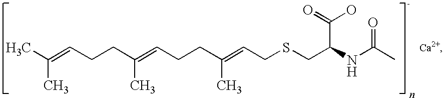

wherein n is within the range of about 1.8-2.2. In some embodiments, n is 2.

In certain embodiments, wherein the compound is of formula Ih and the binding partner is glucosamine, the complex is of formula:

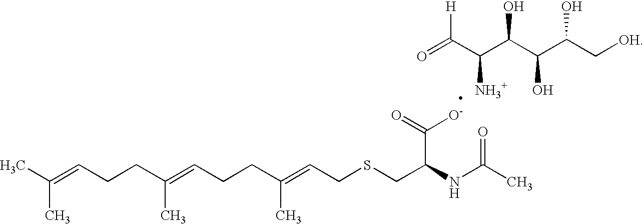

In certain embodiments, wherein the compound is of formula Ih and the binding partner is nicotinamide, the complex is of formula:

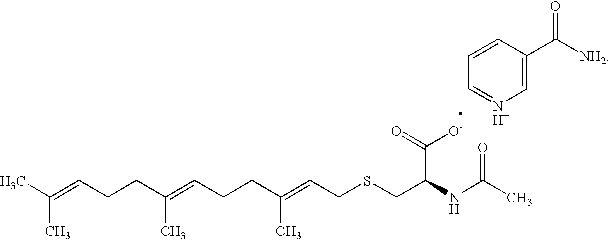

In certain embodiments, wherein the compound is of formula Ih and the binding partner is strontium, the complex is of formula:

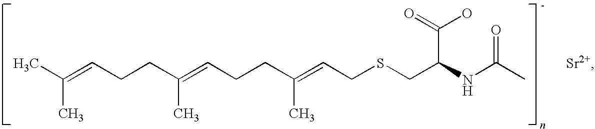

wherein n is within the range of about 1.8-4.2. In some embodiments, n is 4.

In certain embodiments, wherein the compound is of formula Ii and the binding partner is sodium, the complex is of formula:

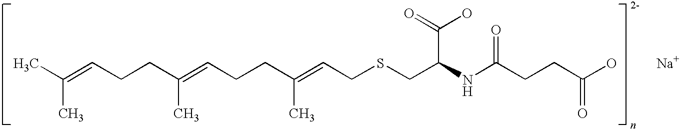

wherein n is within the range of about 0.3 to 1.2. In some embodiments n is within the range of 0.5 to 1. In some embodiments, n is 1. In some embodiments, n is 0.5.

In some embodiments, wherein the compound is of formula Ii and the binding partner is sodium, the complex exists as a hydrate.

The present invention encompasses the surprising finding that certain complexes comprising a compound of formula I have unexpected desirable characteristics. For example, among other things, the present invention demonstrates that complexes comprising a compound of formula Ih with a strontium binding partner (“strontium complex”) is surprisingly more effective at inhibiting inflammation than the complex comprising a compound of formula Ih with a calcium binding partner (“calcium complex”). In certain embodiments, it was found using an MPO Inhibition Protocol (see Example 13), the strontium complex had improved MPO inhibition than the calcium complex. In certain embodiments, it was found using an Edema Inhibition Protocol, that the strontium complex was more effective at decreasing edema than the calcium complex. In certain embodiments, the strontium complex is surprisingly more effective at decreasing sensory irritation than the calcium complex. In certain embodiments, the sensory irritation is pain.

The present invention provides systems for characterizing different complexes as described herein, and optionally for determining their relative activities and/or there activities in relationship to uncomplexed AFC compound and/or to AFC (see, for example, protocols and data presented in the Examples section).

In some embodiments, inventive complexes show anti-inflammatory activity that is at least as potent as that of uncomplexed AFC compound, uncomplexed binding partner, and/or or uncomplexed AFC. In some embodiments, inventive complexes show anti-inflammatory activity that it at least 1.5 fold, 2 fold, 2.5 fold, 3 fold, 2.5 fold, 4 fold, 4.5 fold 5 fold, 5.5 fold, 6 fold, 6.5 fold, 7 fold, 7.5 fold, 8 fold, 8.5 fold, 9 fold, 9.5 fold, 10 fold, 15 fold, 20 fold, 25 fold 30 fold, 40 fold, 50 fold or more potent than the activity of uncomplexed AFC compound, uncomplexed binding partner, and/or or uncomplexed AFC.

The spectroscopic data indicates that in strontium, silver, and zinc complexes of AFC (formula Ih), the metal ion is in close proximity with all three polar moieties of the molecule: carboxylic acid, acetamide, and allylic sulfide. The changes in both chemical shift (2-15 ppm) and line broadening (1-5 Hz) of ¹³C signals adjacent to these moieties are consistent with multiple coordinative bonds between the metal ion and AFC. This finding is consistent with the strong affinity of amides and sulfur containing moieties to bind these and other metal ions, as known in the art. This finding is also consistent with the observed enhanced light and air stability of the AFC silver complex and its solutions, since it is known in the art that ordinary silver salts of carboxylic acids (e.g. silver acetate) are highly photosensitive and readily decompose to form insoluble silver oxide.

### 4. Compositions

In some embodiments, pharmaceutical compositions of the present invention encompass compositions comprising at least one complex of a compound of formula I, II, III, IV, or V and a binding partner, and a pharmaceutically acceptable inert ingredient (i.e., a “carrier”).

In some embodiments, pharmaceutical compositions of the present invention encompass compositions comprising a compound of formula I (e.g., formula Ia, Ib, Ic, Id, Ie, If, Ig, or Ih) or formula II, III, IV, or V, a binding partner and a pharmaceutically acceptable inert ingredient (i.e., a carrier).

In general, one or more complexes of the present invention may be formulated into pharmaceutical compositions that include at least one complex of the invention together with one or more pharmaceutically acceptable carriers, including excipients, such as diluents, binders and the like, and additives, such as stabilizing agents, preservatives, solubilizing agents, and buffers, as desired. Formulation excipients may include polyvinylpyrrolidone, gelatin, hydroxy cellulose, acacia, polyethylene glycol, mannitol, sodium chloride and sodium citrate.

For injection or other liquid formulations, water containing at least one or more buffering constituents is commonly utilized, and stabilizing agents, preservatives and solubilizing agents may also be employed. In some embodiments, a provided pharmaceutical composition is or comprises an isotonic solution.

For solid formulations, any of a variety of thickening, filler, bulking and carrier additives may be employed, such as starches, sugars, fatty acids and the like. For topical administration formulations, any of a variety of creams, ointments, gels, lotions and the like may be employed.

For most pharmaceutical formulations, non-active ingredients will constitute the greater part, by weight or volume, of the preparation. For pharmaceutical formulations, it is also contemplated that any of a variety of measured-release, slow-release or time-release formulations and additives may be employed, so that the dosage may be formulated so as to effect delivery of an inventive complex over a period of time. For example, gelatin, sodium carboxymethylcellulose and/or other cellulosic excipients may be included to provide time-release or slower-release formulations, especially for administration by subcutaneous and intramuscular injection.

In practical use, inventive complexes can be combined as the active ingredient in an admixture with a pharmaceutical carrier according to conventional pharmaceutical compounding techniques. Pharmaceutical compositions for the present invention may be formulated for delivery by any of a variety of routes including, for example, oral, parenteral (including intravenous), urethral, vaginal, nasal, topical (e.g., dermal, transdermal), pulmonary, deep lung, inhalation, buccal, sublingual routes, or the like.

In preparing compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets. Tablets, pills, capsules, and the like may also contain a binder such as gum tragacanth, acacia, corn starch or gelatin; excipients such as dicalcium phosphate; a disintegrating agent such as corn starch, potato starch or alginic acid; a lubricant such as magnesium stearate; and/or a sweetening agent such as sucrose, lactose or saccharin. Capsules may contain, in addition to materials of the above type, a liquid carrier such as a fatty oil.

Tablets may contain inventive complexes in admixture with non-toxic pharmaceutically acceptable additives and/or excipients which are suitable for the manufacture of tablets. Such additives or excipients may be, for example, fillers, wetting agents, inert diluents, such as calcium carbonate, sodium carbonate, lactose, calcium phosphate or sodium phosphate; granulating and noneffervescent disintegrating agents, for example, corn starch or alginic acid; binding agents, for example, starch, gelatin or acacia; and lubricating agents, for example, magnesium stearate, stearic acid or talc.

A tablet may be prepared by traditional methods such as by compressing or molding a powder or granules containing inventive complexes. Compressed tablets may be prepared by compressing, in a suitable machine, inventive complexes in a free-flowing form, such as a powder or granules optionally mixed with a binder, lubricant, inert diluent, and/or surface active/dispersing agent(s). Molded tablets may be made by molding, in a suitable machine, the powdered complexes moistened with an inert liquid binder.

Tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. Tablets also may be coated for controlled delivery. For example, a “delayed release” dosage form releases a product or substance at a time other than promptly after administration. Examples of delayed-release systems include repeat action tablets and capsules, and enteric coated tablets where timed release is achieved by a barrier coating.

For capsule compositions, inventive complexes typically are mixed with an inert solid diluent, for example, calcium carbonate, calcium phosphate or kaolin, or soft gelatin capsules wherein the active ingredient(s) is (are) mixed with water or an oil medium, for example, peanut oil, liquid paraffin, or olive oil.

If in an aqueous solution, inventive complexes may be appropriately buffered by means of saline, acetate, phosphate, citrate, acetate or other buffering agents, which may be at any physiologically acceptable pH, generally from about pH 4 to about pH 7. A combination of buffering agents may also be employed, such as phosphate buffered saline, a saline and acetate buffer, and the like. In the case of saline, a 0.9% saline solution may be employed. In the case of acetate, phosphate, citrate, acetate and the like, a 50 mM solution may be employed. One such preservative that may be employed is 0.05% benzalkonium chloride

Liquid preparations may optionally contain a preservative to prevent the growth of microorganisms. Lyophilized preparations may also be utilized, which are reconstituted, such as with saline, immediately prior to administration, and thus may not require a preservative.

Inventive compositions may be formulated as aqueous suspensions in which inventive complexes are in admixture with excipients additives and/or suitable for the manufacture of aqueous suspensions. Such additives and/or excipients include suspending agents such as, for example, sodium carboxymethylcellulose, methylcellulose, hydroxy propylmethylcellulose, sodium alginate, polyvinylpyrrolidone, gum tragacanth, and gum acacia; dispersing or wetting agents may be a naturally occurring phosphatide such as lecithin, or condensation products of an alkylene oxide with fatty acids, for example, polyoxyethylene stearate, or condensation products of ethylene oxide with long chain aliphatic alcohols, for example, heptadecaethyl eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. Aqueous suspensions also may contain one or more coloring agents, one or more flavoring agents, and one or more sweetening agents, such as sucrose or saccharin.

Compositions of the present invention may be formulated as oily suspensions by suspending inventive complexes in a vegetable oil, for example arachis oil, olive oil, sesame oil or coconut oil, or in a mineral oil, such as liquid paraffin. Such oily suspensions may contain a thickening agent, for example, beeswax, hard paraffin or cetyl alcohol. Sweetening agents, such as those set forth above, and flavoring agents may be added to provide a palatable oral composition. These compositions may be preserved, for example, by the addition of an antioxidant such as ascorbic acid.

Compositions of the invention may be in the form of oil in water emulsions. The oily phase may be a vegetable oil, for example, olive oil or arachis oil, or a mineral oil, for example a liquid paraffin, or a mixture thereof. Suitable emulsifying agents may be naturally occurring gums, for example, gum acacia or gum tragacanth, naturally- occurring phosphatides, for example soy bean, lecithin, and esters or partial esters derived from fatty acids and hexitol anhydrides, for example sorbitan monooleate, and condensation products of the partial esters with ethylene oxide, for example, polyoxyethylene sorbitan monooleate. The emulsions also may contain sweetening and flavoring agents.

Compositions of the invention may be formulated as syrups and elixirs. Syrups and elixirs may be formulated with sweetening agents, for example, glycerol, propylene glycol, sorbitol or sucrose. Such formulations also may contain a demulcent, a preservative, and flavoring and coloring agents. Demulcents are protective agents employed primarily to alleviate irritation, particularly mucous membranes or abraded tissues. A number of chemical substances possess demulcent properties. These substances include the alginates, mucilages, gums, dextrins, starches, certain sugars, and polymeric polyhydric glycols. Others include acacia, agar, benzoin, carbomer, gelatin, glycerin, hydroxyethyl cellulose, hydroxypropyl cellulose, hydroxypropyl methylcellulose, propylene glycol, sodium alginate, tragacanth, hydrogels and the like.

It is also possible and contemplated that inventive complexes may be provided or utilized in a dry and/or particulate form. In some embodiments, particles are between about 0.5 and 6.0 μm, such that the particles have sufficient mass to settle on the lung surface, and not be exhaled, but are small enough that they are not deposited on surfaces of the air passages prior to reaching the lung. Any of a variety of different techniques may be used to make dry powder microparticles, including but not limited to micro-milling, spray drying and a quick freeze aerosol followed by lyophilization. With micro-particles, inventive complexes may be deposited to the deep lung, thereby providing quick and efficient absorption into the bloodstream. Further, with such approach penetration enhancers may not be required, as is sometimes the case in transdermal, nasal or oral mucosal delivery routes. Any of a variety of inhalers can be employed, including propellant-based aerosols, nebulizers, single dose dry powder inhalers and multidose dry powder inhalers. Common devices in current use include metered dose inhalers, which are used to deliver medications for the treatment of asthma, chronic obstructive pulmonary disease and the like. Preferred devices include dry powder inhalers, designed to form a cloud or aerosol of fine powder with a particle size that is always less than about 6.0 μm.

Microparticle size, including mean size distribution, may be controlled by means of the method of making. For micro-milling, the size of the milling head, speed of the rotor, time of processing and the like control the microparticle size. For spray drying, the nozzle size, flow rate, dryer heat and the like control the microparticle size. For making by means of quick freeze aerosol followed by lyophilization, the nozzle size, flow rate, concentration of aerosoled solution and the like control the microparticle size. These parameters and others may be employed to control the microparticle size.

In some embodiments, inventive compositions formulated as dispersible powders and/or granules are suitable for use in preparation of an aqueous suspension, for example, by the addition of water. Inventive complexes in such powders and granules are typically provided in admixture with a dispersing or wetting agent, suspending agent, and one or more preservatives. Suitable dispersing or wetting agents and suspending agents are exemplified by those already mentioned above. Additional excipients or example, sweetening, flavoring and coloring agents also may be present.

In some embodiments, complexes of the present invention may be therapeutically administered by means of an injection, typically a deep intramuscular injection, such as in the gluteal or deltoid muscle, of a time release injectable formulation. In some embodiments, an inventive complex is formulated with a PEG, such as poly(ethylene glycol) 3350, and optionally one or more additional excipients and preservatives, including but not limited to excipients such as salts, polysorbate 80, sodium hydroxide or hydrochloric acid to adjust pH, and the like. In some embodiments, an inventive complex is formulated with a poly(ortho ester), which may be an auto-catalyzed poly(ortho ester) with any of a variable percentage of lactic acid in the polymeric backbone, and optionally one or more additional excipients. In some embodiments, poly (D,L-lactide-co-glycolide) polymer (PLGA polymer) is employed, for example a PLGA polymer with a hydrophilic end group, such as PLGA RG502H from Boehringer Ingelheim, Inc. (Ingelheim, Germany).

Inventive compositions may be prepared, for example, by combining an inventive complex in a suitable solvent, such as methanol, with a solution of PLGA in methylene chloride, and adding thereto a continuous phase solution of polyvinyl alcohol under suitable mixing conditions in a reactor. In general, any of a number of injectable and biodegradable polymers, which are preferably also adhesive polymers, may be employed in a time release injectable formulation. The teachings of U.S. Pat. Nos. 4,938,763, 6,432,438, and 6,673,767, and the biodegradable polymers and methods of formulation disclosed therein, are incorporated herein by reference. The formulation may be such that an injection is required on a weekly, monthly or other periodic basis, depending on the concentration and amount of inventive complex, the biodegradation rate of the polymer, and other factors known to those of skill in the art.

Representative pharmaceutical forms suitable for injectable use include, for example, sterile aqueous solutions or dispersions and sterile powders, such as lyophilized formulations, for the extemporaneous preparation of sterile injectable solutions or dispersions. In all cases, the form should desirably sterile and should be fluid to the extent that it may be administered by syringe. The form must be stable under the conditions of manufacture and storage and may be preserved against the contaminating action of microorganisms such as bacteria and fungi. The carrier can be a solvent or dispersion medium containing, for example, water, ethanol, a polyol, for example glycerol, propylene glycol or liquid polyethylene glycol, suitable mixtures thereof, and vegetable oils.

In general, compositions comprising a therapeutically or pharmaceutically effective amount of an inventive complex may be formulated for administration in unit dosage forms.

Because of their ease of administration, tablets and capsules represent an advantageous oral dosage unit form. If desired, a composition including complex of the invention may be coated by standard aqueous or nonaqueous techniques. In another advantageous dosage unit form, sublingual pharmaceutical compositions may be employed, such as sheets, wafers, tablets or the like. Inventive complexes can also be administered intranasally as, for example, by liquid drops or spray.

Formulations suitable for buccal administration include tablets and lozenges comprising a provided complex in a flavored base, such as sucrose, acacia or tragacanth; and pastilles comprising a provided complex in an inert base, such as gelatin and glycerin or sucrose and acacia.

Formulations of the present invention suitable for topical application to the skin take the form of an ointment, cream, lotion, paste, gel, spray, aerosol, or oil. Additives which may be used include vaseline, lanoline, polyethylene glycols, alcohols, transdermal enhancers, and combinations of two or more thereof.

Formulations suitable for transdermal administration may also be presented as medicated bandages or discrete patches adapted to remain in intimate contact with the epidermis of the recipient for a prolonged period of time. Formulations suitable for transdermal administration may also be delivered by iontophoresis (passage of a small electric current (^{˜}15 mA) to “inject” electrically charged ions into the skin) through the skin. For this, the dosage form typically takes the form of an optionally buffered aqueous solution of the active agent, i.e. a provided complex.

For administration by inhalation, compositions for use in the present invention can be delivered in the form of an aerosol spray in a pressurized package or as a nebulizer, with use of suitable propellants. In the case of a pressurized aerosol, the dosage unit can be determined by providing a valve to deliver a metered dose in accordance with the invention.

Parenterally administered compositions are commonly formulated to allow for injection, either as a bolus or as a continuous infusion. For parenteral application, “parenteral” meaning subcutaneous injections, intravenous, intramuscular, intrasternal injection, or infusion techniques, particularly suitable vehicles consist of solutions, preferably oily or aqueous solutions, as well as suspensions, emulsions, or implants. Formulations for injection can be prepared in unit dosage forms, such as ampules, or in multi-dose units, with added preservatives. Compositions for injection can be in the form of suspensions, solutions, or emulsions, containing either oily or aqueous additives. They may also contain formulatory agents such as suspending agents, stabilizing agents, and/or dispersing agents. Inventive complexes may also be presented in powder form for reconstitution with a suitable vehicle before use.

Compositions of the present invention also may be in the form of a sterile injectable aqueous or oleaginous suspension. Injectable compositions, such as sterile injectable aqueous or oleaginous suspensions, may be formulated according to the known art using suitable dispersing or wetting agents and suspending agents. The sterile injectable composition may also be a sterile injectable solution or suspension in a nontoxic parenterally acceptable diluent or solvent, for example, as a solution in 1,3 butanediol. Among the acceptable vehicles and solvents that may be employed are water, Ringer's solution, and isotonic sodium chloride solution. In some embodiments, formulations of the present invention suitable for parenteral administration conveniently comprise sterile aqueous preparations of inventive complexes, which preparations are preferably isotonic with the blood of the intended recipient. Such preparations may conveniently be prepared by admixing the active compound with water or a glycine buffer and rendering the resulting solution sterile and isotonic with the blood.

In addition, sterile, fixed oils are conventionally employed as a solvent or suspending medium. Aqueous suspensions may contain substances which increase the viscosity of the suspension and include, for example, sodium carboxymethyl cellulose, sorbitol and/or dextran. Optionally, the suspension may also contain stabilizers.

Alternately or additionally, complexes of the present invention can be formulated in a parenteral lipid solution.

### 5. Methods

Among other things, the present invention provides methods of treating, lessening the severity of and/or of delaying onset of one or more symptoms or aspects of inflammation by administering a therapeutically effective amount of an inventive complex.

In some embodiments, inventive complexes are used in the treatment of inflammation (acute or chronic), inflammatory diseases, inflammatory disorders (e.g. asthma, autoimmune diseases, etc.), or inflammatory responses of the immune system. In some embodiments, provided complexes are useful in the manufacture of a medicament used in the treatment of inflammation (acute or chronic), inflammatory diseases, inflammatory disorders (e.g. asthma, autoimmune diseases, etc.), or inflammatory responses of the immune system.

In general, the actual quantity of inventive complex administered to a particular patient will vary depending on the severity and type of indication, the mode of administration, the particular complex used, the formulation used, and the response desired, and may optionally be further influenced by the condition of the patient, including other medications the patient may be receiving, the patient's habits or overall health, etc.

As will be appreciated by those of skill in the art, an appropriate dosage for treatment is administration, by any of the foregoing means or any other means known in the art, of an amount sufficient to bring about the desired therapeutic effect. Thus, a therapeutically effective amount includes an amount of an inventive complex or composition that is sufficient to induce a desired effect, including specifically an anti-inflammation effect. In general, complexes of the invention of this invention are highly active. For example, an inventive complex can be administered at about 10 μg/kg to about 50 mg/kg body weight, depending on the specific complex selected, the desired therapeutic response, the route of administration, the formulation, and other factors known to those of skill in the art.

### (A) Antiinflammatory

In certain embodiments, the present invention relates to a method of treating or lessening the severity of inflammatory diseases or disorders selected from inflammation (acute or chronic), inflammatory diseases or disorders (e.g., asthma, autoimmune diseases, and COPD including emphysema, chronic bronchitis and small airways disease, etc.), inflammatory responses of the immune system, skin diseases or disorders (e.g., reducing acute skin irritation for patients suffering from rosacea, atopic dermatitis, seborrheic dermatitis, psoriasis, irritant contact dermatitis, contact allergy, photosensitivity, contact urticaria, skin abrasion (e.g., as caused by shaving) or any topical pruritis), irritable bowel syndrome (e.g., Crohn's disease and ulcerative colitis, etc.), neurodegenerative disorders (e.g., Parkinson's disease, Alzheimer's disease, Huntington's disease, dementia pugilistica, Pick's disease, guam parkinsonism dementia complex, fronto-temporal dementia, cortico-basal degeneration, pallido-pontal-nigral degeneration, progressive supranuclear palsy, dementia with Lewy bodies (DLB), and multiple system atrophy (MSA)), as well as inflammation associated with spinal cord injury to promote nerve regeneration and inhibition of rejection of genetically engineered cells by the immune sustem during in vivo gene therapy, wherein the method comprises administering to a patient in need thereof a complex or composition of the present invention.

In some embodiments, provided complexes are capable of effectively inhibiting inflammatory responses. Thus, provided complexes are inhibitors of edema, erythema and myeloperoxidase and are therefore useful for treating one or more disorders associated with inflammatory diseases or disorders as described herein. In some embodiments, the present invention provides methods for inhibiting or reducing sensory irritation, erythema, edema or vesiculation by administering an inventive complex as part of a therapeutic regimen. In some embodiments, the sensory irritation is selected from the group consisting of a sting, burn or itch.

In some embodiments, provided anti-inflammatory complexes are capable of effectively inhiting inflammatory responses by decreasing the levels or production of inflammatory mediators such as inflammatory cytokines, for example TNF IL-1α, IL-1β, IL-2, IL-3, IL-4, IL-5, IL-6, IL-7, IL-8, IL-9, IL-10, IL-11, IL-12/IL-23 p40, IL13, IL-17, LI-18, TGF-β, IFN-γ, GM-CSF, Groα, MCP-1 and TNF-α. Thus, provided anti-inflammatory complexes are inhibitors of proinflammatory cytokines and are therefore useful in treating one or more disorders associated with inflammatory diseases, conditions or disorders described herein.

In some embodiments, the treatment of inflammatory diseases or disorders is achieved using provided complexes without having the side effects of corticosteroids or NSAIDS.

In some embodiments, the provided complexes of the present invention are capable of effective inhibiting oxidative burst response from neutrophils. Thus, provided complexes are inhibitors of oxidative burst response and are therefore useful in the treatment or amelioration of symptoms relating to oxidative damage caused by chemical or environmental factor (e.g., UV damage on the skin).

### (B) Immune Stimulatory

In some embodiments, certain complexes of the present invention are capable of promoting inflammatory responses, and are therefore proinflammatory. Thus, provided proinflammatory complexes are promoters of edema, erythema and myeloperoxidase (a marker for neutrophil infiltration) and are therefore useful for treating one or more disorders associated with the suppression of inflammatory responses as described herein. Therefore, such complexes are administered to a subject suffering from or susceptible to one or more diseases, conditions or disorders associated with suppression of inflammatory responses.

In some embodiments, the present invention relates to a method of treating or lessening the severity of diseases, conditions or disorders associated with the suppression of inflammatory responses selected from for example, treatment of secondary bacterial or viral infections inflicting subjects with acquired immune deficiency syndrome (AIDS), suppression of systemic inflammatory response syndromes following severe burn injuries and cardiac surgeries and also the side-effect of a number of drugs, for example thalidomide.

### (C) Skin Conditions

In some embodiments, provided herein is a method for treating, lessening the severity of and/or delaying onset of a skin condition, the method comprising the step of topically applying onto a surface of a subject, including a human, in need thereof, an effective amount of a composition comprising at least one complex as described herein, a carrier and optionally an additional active ingredient. In another aspect, provided herein is a method for treating, lessening the severity of and/or delaying onset of a skin condition, the method comprising the step of topically applying onto a surface of a subject, including a human, in need thereof, a provided complex. In a further embodiment, provided herein is a method of promoting healthy skin in a subject, including a human, in need thereof, the method comprising the step of topically applying onto a surface of a subject, including a human, in need thereof, an effective amount of a composition comprising at least one complex as described herein, a carrier and optionally an additional active ingredient. In a further aspect, provided herein is a method of promoting healthy skin in a subject, including a human, in need thereof, the method comprising the step of topically applying onto a surface of a subject, including a human, in need thereof, a provided complex.

In certain embodiments, the present invention provides for use of a provided complex in the manufacture of a medicament useful for treating a skin condition as described herein.

In some embodiments, the present invention provides a method for treating, lessening the severity of and/or delaying onset of inflammation in a subject, including a human, in need thereof, comprising the step of administering an effective amount of a composition comprising at least one complex as described herein, a carrier and optionally an additional active ingredient. In a further aspect, the present invention provides a method for treating, lessening the severity of and/or delaying onset of inflammation in a subject, including a human, in need thereof, comprising the step of administering a provided complex.

In certain embodiments, the present invention provides uses of provided complexes and/or compositions in the treatment or prevention of diseases, disorders, or conditions associated with suppression of inflammatory responses. In certain embodiments, the present invention provides a composition for treating or preventing conditions associated with suppression of the inflammatory responses in a subject, including a human, in need of treatment, that comprises of at least one complex as described herein, a carrier and optionally, an additional active ingredient. In some embodiments, provided herein is a method for treating, lessening the severity of and/or delaying onset of a disease, disorder, or condition associated with suppression of inflammatory responses in a subject, including a human, in need thereof, the method comprising the step of administering an effective amount of a composition comprising at least one complex as described herein, a carrier and optionally an additional active ingredient. In a further aspect, provided herein is a method for treating, lessening the severity of and/or delaying onset of a disease or condition associated with suppression of inflammatory responses in a subject, including a human, in need thereof, the method comprising the step of administering a provided complex.

Exemplary diseases, disorders or conditions that may be treated with provided complexes in accordance with the present invention are addressed individually below.

### Rosacea

Rosacea is a chronic, inflammatory skin disorder that afflicts about 14 million people in the US (FoxAnalytics, The Dermatology Market Outlook to 2011, B.I. LTD, Editor: London, UK, p. 201; Crandall, M. A. Market Intelligence Report, K. Information, Editor, 2008: New York. p 359). With peak onset between the ages of 51 and 60, its incidence will grow substantially in the years ahead. The condition is characterized by a constellation of symptoms that include central facial erythema, telangiectasias, papules, granulomatous nodules, phyma formation and ocular changes. Flares and remissions occur without rationale. There are no known cures for rosacea. Exemplary cytokines associated with rosacea may include TNFα, ILβ, IL-6, IL-8, MCP-1 and Groα.

### Psoriasis

Psoriasis is a chronic inflammatory skin disease affecting ˜125 million people worldwide and approximately 2-3% of the general population in the US and Europe (Crandall, M. A. Market Intelligence Report, K. Information, Editor, 2008: New York. P. 359; Naldi, L., Curr. Drug Targets Inflamm. Allergy, 2004, 3: 121-128). Although the pathogenesis of psoriasis has not been fully elucidated, recent advances demonstrate targeting key mediators of inflammation as a promising therapeutic approach (Numerof et al., BioDrugs, 2006, 20: 93-103; Menter et al., J. Am. Acad. Dermatol., 2009, 60: 643-659). Direct therapeutic approaches include using antibodies or soluble receptors (i.e., biologics) to directly neutralize the specific cytokine of interest. However, biologic cytokine-derived therapies are expensive to produce, require sustained high blood levels in order to develop significant skin levels, may induce the production of neutralizing antibodies (leading to a diminished response to therapy), and must be administered by injection. Topical treatments have largely been ineffective, so market growth has been driven by systemic agents that have serious potential side effects. Corticosteroids remain the cornerstone of current topical treatment, but they are far from ideal. Long-term steroid use brings safety concerns ranging from issues of systemic absorption to cutaneous atrophy and its various clinical presentations. Today's US market for psoriasis treatments is greatly underserved, as only 60% of sufferers are being treated (Horn et al., J. Am. Acad. Dermatol. 2007, 57: 957-962).

Psoriasis can be conceived in simple terms, as a self reinforcing loop, in which deregulated inflammatory activity stimulates the epidermal Stat3c signaling pathway in the epidermis resulting in epidermal hyperplasia. The affected keratinocytes secrete cytokines which simulate the immune system, including T-helper cell (THc) infiltration and accumulation. Cytokines from the activated immune cells positively feedback on to the epidermal Stat3c pathway maintaining and amplifying the pathophysiology. Inhibition of THc infiltration and accumulation would decrease Stat3c expression and the onset of psoriasis. Exemplary cytokines associated with psoriasis may include TNFα, IL1α, ILβ, IL-2, IL-6, IL-8, IL-12, MCP-1, Groα and IFNγ.

In some embodiments, the present invention provides methods of treating, lessening the severity of and/or delaying onset of inflammatory skin conditions such as psoriasis comprising administering to a subject in need thereof a dosage form comprising a provided complex, wherein cytokine levels and/or activity (e.g., levels and/or activity of one or more of TNFα, IL1α, ILβ, IL-2, IL-6, IL-8, IL-12, MCP-1, Groa and IFNγ) are reduced by more than about 20% as compared to levels and/or activity in a subject who has not been administered a provided complex (e.g., as determined using a K5.Stat3c psoriasis mouse model).

### Atopic Dermatitis

Atopic dermatitis, or eczema, is characterized by chromic inflammation and irritation of the skin. Its causes are varied but immunological in nature. In the US, prevalence is 10% to 20% in children and 1% to 3% in adults. Topical dermatitis is caused by exposure to substances such as poison ivy, detergents and cosmetics that trigger allergic skin reactions. According to present theories, atopic dermatitis is thought to be caused by skin barrier defects that lead to increased exposure to substances such as allergens exposed by inhalation or ingestion. When dermatitis occurs, corticosteroids are the primary treatment. Atopic dermatitis, however, disproportionately affects children, and long-term steroid use in this population raises safety concerns. Exemplary cytokines associated with atopic dermatitis include but are not limited to TNFα, IL1β, IL-6, IL-8, MCP-1, Groα, IL-4, IL-5, IL-10, IL-13, IL-17 and IFNγ.

In some embodiments, the present invention provides methods of treating, lessening the severity of and/or delaying onset of inflammatory skin conditions such as atopic dermatitis comprising administering to a subject in need thereof a dosage form comprising a provided complex, wherein cytokine levels and/or activity (e.g., levels and/or activity of one or more of TNFα, IL1β, IL-6, IL-8, MCP-1, Groα, IL-4, IL-5, IL-10, IL-13, IL-17 and IFNγ) are reduced by more than about 20% as compared to levels and/or activity in a subject who has not been administered a provided complex (e.g., as determined using a ovalbumin-challenged ft/ft atopic dermatitis mouse model).

### Seborrhic Dermatitis

Seborrheic dermatitis, commonly called dandruff, is a disease that causes redness, itchiness, and flaking of the skin. It affects the scalp, face, trunk, and particularly the sebum-gland rich areas of the skin, usually causing the skin to look inflamed and scaly.

Seborrheic dermatitis most often occurs in adults from 30 to 60 years of age and is more common in men than in women. Although the exact cause is not known, those afflicted with seborrhoeic dermatitis often have an unfavorable epidermic response caused by infections. Seborrheic dermatitis has also been linked to neurologic disorders such as Parkinson's disease and epilepsy. The treatment of seborrheic dermatitis depends on its location on the body. Treatment also depends on the person's age. Dandruff is often treated with a shampoo that contains salicylic acid, the prescription medicine selenium sulfide, zinc pyrithione, ketoconazole or coal tar. Steroid lotions may be used in adolescents and adults. Exemplary cytokines associated with seborrhic dermatitis include but are not limited to TNFα, ILβ, IL-6, IL-8, MCP-1, and Groα.

### Inflammatory Cytokines and Rosacea, Psoriasis, Atopic Dermatitis and Seborrhic Dermatitis

As described herein, the present invention provides methods of treating, lessening the severity of and/or delaying onset of inflammatory skin conditions (e.g., rosacea, psoriasis, atopic dermatitis and seborrhic dermatitis).

In some embodiments, the present invention provides methods of treating, lessening the severity of and/or delaying onset of inflammatory skin conditions (e.g., rosacea, psoriasis, atopic dermatitis and seborrhic dermatitis) by administering a provided complex or composition thereof. In certain embodiments, the present invention provides methods of treating, lessening the severity of and/or delaying onset of inflammatory skin conditions (e.g., rosacea, psoriasis, atopic dermatitis and seborrhic dermatitis) by administering a provided complex or composition thereof.

According to one aspect, the present invention provides methods of treating, lessening the severity of and/or delaying onset of inflammatory skin conditions (e.g., rosacea, psoriasis, atopic dermatitis and seborrhic dermatitis) comprising administering to a subject in need thereof a dosage form comprising a provided complex, wherein inflammatory activity (e.g., MPO activity) is reduced by more than about 30% as compared to control (e.g., as determined using an MPO activity assay).

According to one aspect, the present invention provides methods of treating, lessening the severity of and/or delaying onset of inflammatory skin conditions (e.g., rosacea, psoriasis, atopic dermatitis and seborrhic dermatitis) comprising administering to a subject in need thereof a dosage form comprising a provided complex, wherein inflammatory activity (e.g., MPO activity) is reduced by more than about 60% as compared to control (e.g., as determined using an MPO activity assay).

According to one aspect, the present invention provides methods of treating, lessening the severity of and/or delaying onset of inflammatory skin conditions (e.g., rosacea, psoriasis, atopic dermatitis and seborrhic dermatitis) comprising administering to a subject in need thereof a dosage form comprising a provided complex, wherein inflammatory activity (e.g., erythema activity) is reduced by more than about 30% as compared to control (e.g., as determined using an erythema activity assay).

According to one aspect, the present invention provides methods of treating, lessening the severity of and/or delaying onset of inflammatory skin conditions (e.g., rosacea, psoriasis, atopic dermatitis and seborrhic dermatitis) comprising administering to a subject in need thereof a dosage form comprising a provided complex, wherein inflammatory activity (e.g., edema activity) is reduced by more than about 30% as compared to control (e.g., as determined using an edema activity assay).

In some embodiments, the present invention provides methods of treating, lessening the severity of and/or delaying onset of inflammatory skin conditions (e.g., rosacea, psoriasis, atopic dermatitis and seborrhic dermatitis) comprising administering to a subject in need thereof a dosage form comprising a provided complex, wherein cytokine levels and/or activity (e.g., levels and/or activity of one or more of TNF-α, IL-1β, IL-8, IL-6, MCP-1, and Groα) are reduced by more than about 20% as compared to levels and/or activity in a subject who has not been administered said dosage form (e.g., as determined using a TPA-induced mouse ear inflammatory model).

In some embodiments, the present invention provides methods of treating, lessening the severity of and/or delaying onset of inflammatory skin conditions (e.g., rosacea, psoriasis, atopic dermatitis and seborrhic dermatitis) comprising administering to a subject in need thereof a dosage form comprising a provided complex, wherein cytokine levels and/or activity (e.g., levels and/or activity of one or more of TNF-α, IL-1β, IL-8, IL-6, MCP-1, and Groα) are reduced by more than about 20% as compared to levels and/or activity in a subject who has not been administered said dosage form (e.g., as determined using an LPS-TLR4-induced cytokine release inflammatory model in HMEC-1 cell line).

In some embodiments, the present invention provides methods of treating, lessening the severity of and/or delaying onset of inflammatory skin conditions (e.g., rosacea, psoriasis, atopic dermatitis and seborrhic dermatitis) comprising administering to a subject in need thereof a dosage form comprising a provided complex, wherein cytokine levels and/or activity (e.g., levels and/or activity of one or more of TNF-α, IL-1β, IL-8, IL-6, MCP-1, and Groα) are reduced by more than about 20% as compared to levels and/or activity in a subject who has not been administered said dosage form (e.g., as determined using an ATPγS-purinergic receptor-induced cytokine release inflammatory model in HMEC-1 cell line).

In some embodiments, the present invention provides methods of treating, lessening the severity of and/or delaying onset of inflammatory skin conditions (e.g., rosacea, psoriasis, atopic dermatitis and seborrhic dermatitis) comprising administering to a subject in need thereof a dosage form comprising a provided complex, wherein cytokine levels and/or activity (e.g., levels and/or activity of one or more of TNF-α, IL-1β, IL-8, IL-6, MCP-1, and Groα) are reduced by more than about 20% as compared to levels and/or activity in a subject who has not been administered said dosage form (e.g., as determined using a TPA-induced cytokine release inflammatory model in NHEK cell line).

In some embodiments, the present invention provides methods of treating, lessening the severity of and/or delaying onset of inflammatory skin conditions (e.g., rosacea, psoriasis, atopic dermatitis and seborrhic dermatitis) comprising administering to a subject in need thereof a dosage form comprising a provided complex, wherein cytokine levels and/or activity (e.g., levels and/or activity of one or more of TNF-α, IL-1β, IL-8, IL-6, MCP-1, and Groα) are reduced by more than about 20% as compared to levels and/or activity in a subject who has not been administered said dosage form (e.g., as determined using a TNFα-induced cytokine release inflammatory model in HUVEC cell line).

(D) Sun Screen (Protection from UV Damage)

Oxidative stresses caused by environmental insults such as ultraviolet (“UV”) rays from the sun, chemical irritants, cigarette smoke exposure, consumption of foods with high saturated fat and environmental pollutants as well as the natural process of aging, contributing to the generation of free radicals and reactive oxygen species (“ROS”), stimulate inflammatory responses, especially in the skin (Pilla et al. Intl J. Cosm. Sci. 2005 v 27 p 17-34). High levels of ROS contribute to adverse effects on the skin including erythema, edema, photoaging and skin cancer (Trouba et al. Antioxid. Redox Signal 2002 v 4 p 665-673). Neutrophil infiltration during inflammatory responses is associated with increased oxygen consumption and generation of ROS. Extracellular inflammatory agonists such as fMLP bind to GPCRs sich as formyl peptide receptors (“FPR”), to trigger the oxidative burst response (i.e., the rapid rapid release of ROS). In some instances, sun screening agents absorbed into the skin lead to an increase in reactive oxygen species.

In certain embodiments, the present invention provides methods of treating, lessening the severity of and/or delaying onset of UV damage to especially the skin of a subject in need thereof, by administering a provided complex or composition thereof. According to one aspect, the present invention provides methods of treating, lessening the severity of and/or delaying onset of UV damage to especially the skin of a subject, in need thereof, comprising administering to a subject in need thereof a dosage form comprising a provided complex which inhibits more than about 20% of superoxide formation.

In certain embodiments, the present invention provides methods of decreasing the amount of reactive oxygen species in a cell, comprising the step of contacting the cell with a provided complex, wherein the complex inhibits more than about 20% of superoxide formation. In certain embodiments, the present invention provides methods of decreasing the amount of reactive oxygen species in a subject in need thereof, comprising the step of administering to the subject a provided complex, wherein the complex inhibits more than about 20% of superoxide formation.

In certain embodiments, the present invention provides for use of a provided complex in the manufacture of a sun-screening composition.

In certain embodiments, one or more provided complexes are used in combination with one or more conventional sun-screening agents. In certain embodiments, the present invention provides a method of using one or more provided complexes in combination with one or more conventional sun-screening agents, wherein the complex inhibits more than about 20% of superoxide formation.

### (E) Antimicrobial Effects

In some embodiments, the present invention provides methods of treating, lessening the severity of and/or delaying onset of the symptoms of epithelial-related conditions, caused or aggravated by bacteria in animals, particularly humans, in need thereof. In some embodiments, provided methods are useful for epithelial-related conditions (e.g. skin conditions, respiratory conditions, nasal conditions, ocular conditions, oral conditions, conditions of the external ear, vaginal conditions, genitourinary conditions, rectal conditions, bacterial-related conditions of similar tissues, etc.).

In some embodiments, skin conditions include cellulitis; erysipelas; impetigo; ecthyma; cutaneous anthrax; necroticizing fasciitis; toe web infections; sycosis barbae; furuncles and carbuncles; Staphylococcal scalded skin syndrome; blistering distal dactylitis; acute paronychia; folliculitis; acne vulgaris; cutaneous diphtheria; erythrasma; bacterial colonization of open wounds (e.g., cuts, lesions, scrapes, burns, lacerations, chronic wounds, infected animal bites, etc.).

In some embodiments, respiratory conditions include pneumonia; hypersensitivity pneumonitis; upper and lower respiratory tract infections (e.g., secondary bacterial infections in chronic bronchitis, asma, etc.); chronic obstructive pulmonary disease; diphtheria; bronchopulmonary dysplasia; pertussis; legionellosis (e.g., Legionnaires' disease, Pontiac fever; pharyngitis, etc.).

In some embodiments, nasal conditions include bacterial rhinitis; paranasal sinusitis, etc. In some embodiments, ocular conditions include chronic blepharitis; endophthalmitis, etc. In some embodiments, oral conditions include gingivitis; dental caries; early childhood caries, etc. In some embodiments, conditions of the external ear include otitis media, etc.

In some embodiments, vaginal conditions include bacterial vaginosis; chanchroid; syphilis; donovanosis; gonorrhea; lymphogranuloma venereum; non-gonococcal urethritis; staphylococcal infection, etc.

In some embodiments, bacteria include Gram positive bacteria, Gram negative bacteria. Particularly relevant bacteria include for example Actinobacillus sp. (e.g., Actinobacillus pleuropneumoniae, etc.); Actinomyces sp. (e.g., Actinomyces israelli, etc.); Bacillus sp. (e.g., Bacillus anthracis, etc.); Bordatella sp. (e.g., Bordatella pertussis, etc.); Branhamella (Moraxella) sp. (e.g., Branhamella catarrhalis, etc.); Calymmatobacterium sp. (e.g., Calymmatobacterium granulomatis, etc.); Chlamydia sp. (e.g., Chlamydia trachomatis, etc.); Chlamydophila sp. (e.g., Chlamydophila pneumoniae, etc.); Corynebacterium sp. (e.g., Corynebacterium diphtheriae, etc.); Eikenella sp. (e.g., Eikenella corrodens, etc.); Enterobacter sp. (e.g., Enterobacter aerogenes, Enterobacter cloacae, etc.); Enterococcus sp.(e.g., Enterococcus faecalis, etc.); Escherichia sp. (e.g., Escherichia coli, etc.); Fusobacterium sp. (e.g., Fusobacterium nucleatum, etc.); Gardnerella sp. (e.g., Gardnerella vaginalis, etc.); Granuloma sp. (e.g., Granuloma inguinale, etc.); Haemophilus sp. (e.g., Haemophilus influenza, Haemophilus ducreyi, etc.); Histophilus sp. (e.g., Histophilus somnus, etc.); Klebsiella sp. (e.g., Klebsiella pneumoniae, etc.); Legionella sp. (e.g., Legionella pneumophila, etc.); Mannheimia sp. (e.g., Mannheimia haemolytica, etc.); Mobiluncus sp. (e.g., Mobiluncus curtisii, Mobiluncus mulieris, etc.); Mycobacterium sp. (e.g., Mycobacterium immunogenum, Mycobacterium tuberculosis, etc.); Mycoplasma sp. (e.g., Mycoplasma pneumonia, Mycoplasma hyopneumoniae, Mycoplasma gallisepticum, Mycoplasma synoviae, Mycoplasma meleagridis, Mycoplasma gallinarum, Mycoplasma anatis, Mycoplasma hominis, etc.); Neisseria sp. (e.g., Neisseria gonorrhoeae, etc.); Nocardia sp. (e.g., Nocardia asteroides, Nocardia brasiliensis, Nocardia caviae, etc.); Ornithobacterium sp. (e.g., Ornithobacterium rhinotracheale, etc.); Pasteurella sp. (e.g., Pasteurella multocida, etc.); Pneumocystis sp. (e.g., Pneumocystis carinii, etc.); Prevotella sp. (e.g., Prevotella melaninogenica, Prevotella intermedia, etc.); Propionibacterium sp. (e.g., Propionibacterium acnes, Propionibacterium propionicus, Propionibacterium freudenreichii, etc.); Proteus sp. (e.g., Proteus vulgaris, Proteus mirabilis, Proteus penneri, etc.); Psuedomonas sp. (e.g., Psuedomonas aeruginosa, etc.); Staphylococcus sp. (e.g., Staphylococcus aureus, Staphylococcus pseudintermedius, Staphylococcus epidermidis, Staphylococcus saprophyticus, etc.); Streptococcus sp. (e.g., Streptococcus pneumoniae, Streptococcus mutans, Streptococcus mitis, Streptococcus salivarius, etc.); Treponema sp. (e.g., Treponema pallidum, etc.); Ureaplasma sp. (e.g., Ureaplasma urealyticum, etc.); Vibrio sp. (e.g., Vibrio vulnificus, etc.); Yersinia sp. (e.g., Yersinia pestis, etc.), etc.

In some embodiments, epithelial-related conditions may be associated with clinical indications (e.g., infection). In some embodiments, epithelial-related conditions may not be associated with clinical indications (e.g., infection). In some embodiments, epithelial-related conditions are associated with clinical indications (e.g., infection).

In some embodiments, methods of the present invention are useful in treating, lessening the severity of and/or delaying onset of epithelial-related conditions caused or aggravated by bacteria in animals, including humans, in need thereof. In some embodiments, methods of the present invention are useful in treating epithelial-related conditions in animals, including veterinary animals, in need thereof.

In some embodiments, methods described herein comprise a step of administering to an animal, including a human, in need thereof, an effective amount of a provided complex.

In some embodiments, provided herein, is a method for disinfection of a surface such as skin or surface of a medical device, etc.

In certain embodiments, the present invention provides for use of a provided complex in the manufacture of a medicament useful for treating an epithelial-related condition caused or aggravated by bacteria.

In some embodiments, provided methods kill, inactivate, inhibit the growth of and/or decolonize bacteria on a surface. In some embodiments, provided methods are useful in killing, inactivating, inhibiting the growth of and/or decolonizing bacteria in biofilms on a surface. In some embodiments, provided methods are useful in preventing growth or colonization of bacteria to form biofilms on a surface.

Although not wishing to be bound by one theory, it is believed that bacterial challenge triggers certain signal transduction cascades eliciting certain immune and/or inflammatory responses, which result in the release of a set of inflammatory mediators, such as cytokines and chemokines. In some embodiments, provided complexes and/or compositions thereof modulate levels of inflammatory mediators, for example, cytokines. Non-limiting examples of inflammatory mediators modulated by provided complexes and compositions include but are not limited to IL-1α, IL-1β, IL-2, IL-3, IL-4, IL-5, IL-6, IL-7, IL-8, IL-9, IL-10, IL-11, IL-12/IL-23 p40, IL13, IL-17, IL-18, TGF-β, IFN-γ, GM-CSF, GRoα, MCP-1 and TNF-α.

Although not wishing to be bound by one theory, it is believed that provided complexes and compositions thereof modulate levels of inflammatory mediators that are associated with a variety of signal transduction pathways. Non-limiting examples of signal transduction pathways that result in release of inflammatory mediators such as cytokines, include but are not limited to G-protein-mediated, PPAR-mediated, Toll-like receptors-mediated, and TNF-α receptor-mediated. Although not wishing to be bound by one theory, it is believed that provided complexes and compositions thereof modulate T-helper cell infiltration and accumulation.

In some embodiments, complexes of the present invention are capable of effectively inhibiting inflammatory responses triggered by bacterial challenge by decreasing levels or production of inflammatory mediators such as inflammatory cytokines, for example TNF IL-1α, IL-1β, IL-2, IL-3, IL-4, IL-5, IL-6, IL-7, IL-8, IL-9, IL-10, IL-11, IL-12/IL-23 p40, IL13, IL-17, IL-18, TGF-β, IFN-γ, GM-CSF, Groα, MCP-1 and TNF-α.

In some embodiments, an inventive complex is capable of effectively inhibiting inflammatory responses that are triggered by bacterial challenges. Thus, provided complexes are inhibitors of infiltration and activation of inflammatory cells such as neutrophils (as measured by the activity of myeloperoxidase (MPO)), lymphocytes, monocytes, mast cells, etc., and/or inhibitors of expression and activation of cell surface adhesion molecules (e.g. VCAM-1 and ICAM-1), and are therefore useful for treating one or more conditions caused or aggravated by bacteria and associated with inflammation, as described herein.

In general, the actual quantity of inventive complexes administered to a particular patient will vary depending on the severity and type of indication, the mode of administration, the particular complex used, the formulation used, and the response desired, and may optionally be further influenced by the condition of the patient, including other medications the patient may be receiving, the patient's habits or overall health, etc.

As will be appreciated by those of skill in the art, an appropriate dosage for treatment is administration, by any of the foregoing means or any other means known in the art, of an amount sufficient to bring about the desired therapeutic effect. Thus, a therapeutically effective amount includes an amount of an inventive complex or composition that is sufficient to induce a desired effect, including specifically an anti-bacterial effect, an anti-inflammation effect or an anti-bacterial and an anti-inflammation effect. In general, complexes of the invention of this invention are highly active. For example, an inventive complex can be administered at about 10 μg/kg to about 50 mg/kg body weight, depending on the specific complex selected, the desired therapeutic response, the route of administration, the formulation and other factors known to those of skill in the art.

### Acne Vulgaris

Acne vulgaris (acne) is one of the most common skin disorders, affecting about 40-50 million people in the United States (James, W. D., N Engl J Med, 2005, 352: 1463-1472). The etiology of acne is now believed to involve genetic, hormonal, microbiological as well as immunological mechanisms (reviewed in Akhavan et al., Am J Clin Dermatol, 2003, 4: 473-492). The pathogenesis of acne is initiated by the follicular occlusion of adherent keratinocytes and hormone-triggered secretion of sebum resulting in the formation of pathophysiological microstructures called microcomedomes. These may enlarge to form visible non-inflammatory acne lesions, often referred to as open or closed comedomes. Conversion of such non-inflammatory acne lesions to an inflamed acne stage occurs principally as a result of the colonization of microcomedomes and comedomes with Propionibacterium acnes, an aerotolerant anerobic Gram-positive bacterium, which is largely commensal and constitutes a part of the human skin flora. Exemplary inflammatory mediators, for example cytokines whose levels may be elevated during the inflamed stages of acne (upon P. acnes colonization of microcomedomes and comedones) include TNFα, ILβ, IL-6, IL-8, MCP-1 and Groα.

The most common topical acne treatment options include topical antibiotics, topical retinoids, benzoyl peroxide, salicylic acid, sulfur and azelaic acid, which either have anti-bacterial effects or anti-inflammatory effects but not both. In addition, the most-commonly-used anti-inflammatory treatment options for acne have little no effect on inflammatory mediator release.

Surprisingly, the inventors have discovered that certain complexes of the present invention exhibit an anti-bacterial effect and a bacteria-triggered anti-inflammatory effect and are therefore useful in the treatment, prevention and/or amelioration of symptoms of acne. In such embodiments, inventive complexes are therefore considered to be anti-acne agents. In some embodiments, certain complexes of the present invention exhibit an anti-inflammatory effect, wherein the level of inflammatory mediators is inhibited. In some embodiments, some complexes of the present invention have a superior anti-bacterial effect when compared to other anti-bacterial compounds known in the art, such as benzoyl peroxide (BPO).

According to one aspect, the present invention provides methods of treating, lessening the severity of and/or delaying onset of one or more symptoms of an epithelial-related condition, such as acne vulgaris, caused and aggravated by bacteria, such as Propionibacterium acnes in a subject in need thereof, wherein the method comprises the step of administering to a subject in need thereof a therapeutically effective dose of provided complex, having an anti-bacterial effect, as measured by the IC50 on bacterial growth of less than about 300 μg/mL. In certain embodiments, the present invention provides methods of treating, lessening the severity of and/or delaying onset of one or more symptoms of an epithelial-related condition, such as acne vulgaris, caused and aggravated by bacteria, such as Propionibacterium acnes in a subject in need thereof, wherein the method comprises the step of administering to a subject in need thereof a therapeutically effective dose a provided complex, having an anti-bacterial effect, as measured by the minimum bactericidal concentration of less than about 200 μg/mL.

According to one aspect, the present invention provides methods of treating, lessening the severity of and/or delaying onset of one or more symptoms of an epithelial-related condition, such as acne vulgaris, caused and aggravated by bacteria, such as Propionibacterium acnes in a subject in need thereof, wherein the method comprises of the step of administering to a subject in need thereof a therapeutically effective dose of a provided complex, having an anti-inflammatory effect, as exemplified by the inhibition of neutrophil infiltration, as measured by an inhibition of more than about 30% in an MPO activity assay, as determined using an in vivo mouse ear model in which inflammation is induced by P. acnes challenge. In certain embodiments, the present invention provides methods of treating, lessening the severity of and/or delaying onset of one or more symptoms of an epithelial-related condition, such as acne vulgaris, caused and aggravated by bacteria, such as Propionibacterium acnes in a subject in need thereof, wherein the method comprises of the step of administering to a subject in need thereof a therapeutically effective dose of a provided complex, having an anti-inflammatory effect, as exemplified by inhibition of inflammatory mediator release, as measured by an inhibition of more than about 30% in a mediator release assay, as determined using mouse ear model or cell-based models. Exemplary inflammatory mediators, such as cytokines, include IL-6, TNF-α, IL-8. IL-1β, MCP-1 and Groα.

### (F) Joint Inflammation

Inflammation can result from any of a wide variety of skeletal diseases, such as joint-related diseases. Diseases involving inflammation of the bone and joints include (a) arthritis, including, but not limited to, psoriatic arthritis, osteoarthritis, rheumatoid arthritis, juvenile rheumatoid arthritis, juvenile psoriatic arthritis, and gouty arthritis, (b) soft tissue rheumatic diseases, which are rheumatic diseases that affect the tissues and structures that surround a joint and produce pain, swelling or inflammation, such as tendonitis, bursitis, and myofascial syndrome, (c) Reiter's syndrome, a triad of disorders that can appear consecutively or concurrently that include inflammation of the urethra, the iris and ciliary body, and the joints (d) Paget's disease, a metabolic bone disease that involves bone destruction and regrowth which results in deformity, (e) Still's disease, in adults, an illness with fever, rash, and joint pain, which can lead to chronic arthritis; it is more common in children, where it is called systemic juvenile rheumatoid arthritis, (f) sarcoidosis, an immune system disorder characterized by non- necrotising granulomas (small inflammatory nodules) that can affect any organ, though the lungs and lymph nodes appear most often affected, (g) Marfan syndrome, a connective tissue multisystemic disorder characterized by skeletal changes (arachnodactyly, long limbs, joint laxity, pectus), cardiovascular defects (aortic aneurysm which may dissect, mitral valve prolapse), and ectopia lentis (h) Lyme disease, an inflammatory disorder caused by infection with Borrelia burgdorferi, a nonpyogenic spirochete, (i) lupus, any of several forms of ulcerative skin diseases, including, e.g., systemic lupus erythematosus (SLE) and juvenile SLE, (j) gout, (k) polymyalgia rheumatica, a syndrome within the group of collagen diseases involving pain and stiffness in the hip or shoulder area, (1) fibromyalgia, (m) Ehlers-Danlos syndrome, a group of inherited disorders of the connective tissue, occurring in at least ten types, I to X, based on clinical, genetic, and biochemical evidence, varying in severity from mild to lethal, and often characterized by hyperelasticity and fragility of the skin, hypermobility of the joints, and fragility of the cutaneous blood vessels, (n) dermatomyositis, a connective-tissue disease that is characterized by inflammation of the muscles and the skin, (o) polymyositis, a chronic, progressive inflammatory disease of skeletal muscle, (p) scleroderma, a chronic hardening and thickening of the skin, a finding in various different diseases, occurring in a localized or focal form as well as a systemic disease, (q) spondyloarthropathy, any of several diseases affecting the joints of the spine such as ankylosing spondylitis, (r) Behcet's disease, a chronic condition that causes canker sores or ulcers in the mouth and on the genitals, and inflammation in parts of the eye, (s) avascular necrosis, a disease resulting from the temporary or permanent loss of the blood supply to the bones, (t) psoriasis, (u) skin cancer, and (v) bone cancer.

According to one aspect, the present invention provides methods of treating, lessening the severity of and/or delaying onset of one or more symptoms of bone and/or joint inflammation in a subject in need thereof, wherein the method comprises of the step of administering to a subject in need thereof a therapeutically effective dose of a provided complex. In certain embodiments, the present invention provides methods of treating, lessening the severity of and/or delaying onset of one or more symptoms of bone and/or joint inflammation in a subject in need thereof, wherein the method comprises of the step of administering to a subject in need thereof a therapeutically effective dose of a provided complex, having an anti-inflammatory effect, as exemplified by the inhibition of inflammatory mediator release, as measured by an inhibition of more than about 30% in a mediator release assay, as determined using mouse ear model or cell-based models. Exemplary inflammatory mediators, such as cytokines, include TNF, IL-1, IL-4, IL-6, IL-8, IL-12, and interferon-gamma.

In certain embodiments, the present invention provides for use of a provided complex in the manufacture of a medicament useful for treating joint inflammation.

### (G) Wound Healing

Skin wound healing involves a number of phases: inflammation, first with neutrophil and later monocyte/macrophage inflammation, new tissue formation, including matrix formation and differentiation of a neoepithelium, and finally remodeling and maturation. The initial inflammatory phase allows clot formation, controls infection, and promotes vascularization, and produces growth factors. If not controlled properly, the inflammation can lead to pathological healing, e.g., ulcers or scars.

Fibroblasts deposit provisional matrix or granulation tissue, while the newly formed provisional matrix is later degraded in a tissue remodeling process. Degradation of extracellular matrix is mediated by proteases, such as matrix metalloproteases (MMP), gelatinase, and collagenase, as well as protease inhibitors. An imbalance in matrix formation and degradation leads, at one extreme, to chronic ulcers and, on the other extreme, to fibrosis. For example, keloids, an “overhealed response,” are fibrous tissue outgrowths (Michalik, et al. (2001) J. Cell Biol. 154:799-814; Okada, et al. (1997) J. Cell Biol. 137:67-77; Fedyk, et al. (2001) J. Immunol. 166:5749-5755; Ravanti and Kahari (2000) Int. J. Mol. Med. 6:391-407; Peled, et al. (2000) Clin. Past. Surg. 27:489-500).

In wound healing, cells such as platelets, monocyte/macrophages, T cells, and other immune cells, infiltrate the wound and produce factors that regulate growth of tissue. These factors include TGF, tumor necrosis factor (TNF), IL-1, IL-4, IL-6, oncostatin M, GRO-alpha, various angiogenic factors, and chemokines. In turn, these factors stimulate, for expression of, e.g., extracellular matrix and tissue inhibitor of metalloproteases (Ihn and Tamaki (2000) J. Immunol. 165:2149-2155; Feugate, et al. (2002) J. Cell. Biol. 156:161-172). Myofibroblasts, cells that are fibrogenic, are important for wound closure and contraction. Disease states characterized by accumulation of myofibroblasts include pulmonary fibrosis and scleroderma (Feugate, et al. (2002) J. Cell Biol. 156:161-172).

Wound healing of skin and other tissues is a complex process involving proliferation and migration of immune cells, endothelial cells, fibroblasts, stromal cells, myofibroblasts, smooth muscle cells, pericytes, and keratinocytes.

Parameters used to measure healing include rate of healing, breaking strength of healed wounds, degree of epithelialization, thickness of granulation tissue, and density of extracellular matrix (Matsuda, et al. (1998) J. Exp. Med. 187:297-306).

Glucocorticoids are known in the art to interfere with wound healing. In certain embodiments, provided complexes do not interfere with wound healing. In some embodiments, provided complexes interfere with wound healing to a lesser extent than glucocorticoids, and as such are advantageous as compared to glucocorticoids.

According to one aspect, the present invention provides a method of administering a provided complex to a subject, wherein the complex interferes with wound healing to a lesser extent than a standard (e.g., a glucocorticoid).

Parameters and endpoints used to assess wound healing and response to therapeutic, pharmacological, and diagnostic agents, include a number of histological, physiological, and biochemical parameters, e.g., infiltration, activation, or differentiation of neutrophils, monocytes, and macrophages, e.g., differentiation of monocytes to reparative macrophages, and appearance of new stroma, blood vessels, and nerves. Suitable parameters also include expression levels of signaling agents, e.g., transforming growth factor, interleukin-1, and insulin-like growth factor. Measures of epithelization, e.g., rate and thickness, migration of epidermal cells, granulation thickness, degradation and maturation of extracellular matrix, e.g., provisional matrix versus collagenous matrix, wound strength (breaking strength), and fibroblast proliferation rate and phenotype, are also suitable parameters. Increased granulation tissue thickness can resulting stronger healed wounds (see, e.g., Singer and Clark, supra, Werner and Grose (2002) Physiol. Rev. 83:835-870; Matsuda, et al. (1998) J. Exp. Med. 187:297-306; Wankell, et al. (2001) EMBO J. 20:5361-5372).

### (H) Skin Thinning

Skin thinning is a major adverse effect of chronic topical glucocorticoid use (Schwartz et al., J Invest Dermatol, 1994, 102: 241-246). In some embodiments, certain complexes of the present invention do not cause skin thinning. In some embodiments, certain complexes of the present invention cause less skin thinning relative to a standard (e.g., a glucocorticoid). In certain embodiments, provided complexes are therefore considered safer than topical glucocorticoids. According to one aspect, the present invention provides methods of treating, lessening the severity of and/or delaying onset of one or more symptoms of inflammation in a subject in need thereof, wherein the method comprises the step of administering to a subject in need thereof a therapeutically effective dose of provided complex, wherein the provided complex causes less skin thinning relative to a standard (e.g., a glucocorticoid). In certain embodiments, the present invention provides methods of treating, lessening the severity of and/or delaying onset of one or more symptoms of inflammation in a subject in need thereof, wherein the method comprises the step of administering to a subject in need thereof a therapeutically effective dose of provided complex, wherein the provided complex causes less skin thinning than a topical glucocorticoid.

### (I) Cosmetic Uses

Provided complexes may also be useful in the following: reducing fine lines and wrinkles, as anti-dandruff agents, in the treatment of skin disorders due to exposure to UV radiation, combating aging of the skin (e.g., light-induced or chronological aging), for delaying the onset of or repairing stretch marks, in treating or delaying the onset of alopecia of various origins, hair uses (e.g., hair relaxant, reducing irritation and/or inflammation of the scalp, adding natural shine, detangling, adding hair elasticity, hair moisturizer, restoring damaged, e.g, cysteine-depleted hair).

External insults such as pollution, UV radiation, chemical irritants, cigarette smoke, motor vehicle emissions, lotions, cosmetics, etc., cause an increase in the number of free radicals on the skin. Once formed, these highly reactive free radicals can enter the body and deplete electrons from healthy cells, causing inflammation. If left unchecked, increased free radicals could lead to the chronic inflammatory response that could likely cause skin aging, and contribute to premature appearance of fine lines and wrinkles. Provided complexes can protect the skin from UV damage, reverse skin aging and reduce the appearance of fine lines and wrinkles by targeting two key players in the aging process—free radicals, such as superoxide, and inflammation. In certain embodiments, the present invention provides a method of reducing fine lines and wrinkles on a subject in need thereof, comprising the step of administering to the subject a provided complex. In certain embodiments, the present invention provides a method of treating, lessening the severity of, or delaying the onset of a skin disorder caused by UV radiation in a subject in need thereof, comprising the step of administering to the subject a provided complex. In certain embodiments, the present invention provides a method of treating, lessening the severity of, or delaying the onset of aging of the skin in a subject in need thereof, comprising the step of administering to the subject a provided complex. In certain embodiments, the aging is light-induced. In certain embodiments, the aging is chronological aging.

Alopecia areata (AA) is a condition affecting humans, in which hair is lost from some or all areas of the body, usually from the scalp. AA affect ˜0.2% of the population and its incidence has been growing for the last few decades. AA is a T-cell mediated disease of the hair follicle. Predicted mechanisms of action include diversion of the T-cell response from the hair follicle to the epidermis, interference with lymphocyte homing, induction of nonspecific, localized immunosuppression as a result of a chronic immune response, and production of immunosuppressive cytokines (e.g. TGF-β and IL-10). In certain embodiments, the present invention provides a method of treating, lessening the severity of, or delaying the onset of alopecia in a subject in need thereof, comprising the step of administering to the subject a provided complex.

Skin stretching creates inflammatory reactions, and inflammatory cells activate skin matrix degradation to form stretch marks. Down-regulation of pro-inflammatory cytokines, such as IL-1a , IL-1b, IL-8, and TNF-a, could repair, lessen the severity of, or delay onset of stretch marks. In certain embodiments, the present invention provides a method of repairing, lessening the severity of, or delaying the onset of stretch marks in a subject in need thereof, comprising the step of administering to the subject a provided complex.

Cysteine bonds are responsible for toughness and overall abrasion resistance of hair. The cysteine bond, also known as disulfide bond, is formed by cross-links between cysteine residues found in the main polypeptide chains that make up hair. Such disulfide bonds that are perpendicular to the axis of the hair and connect the polypeptide chains hold the hair fibers together and contribute to hair's toughness and abrasion resistance. The major metabolite of AFC compounds and complexes is farnesylated cysteine. Farnesylated cysteine can be used by the cellular machinery of hair follicles as a source of cysteine. Thus, AFC complexes may be used in the treatment of cysteine depleted hair such as damaged hair and split ends.

In some embodiments, the present invention provides a hair product comprising a provided complex. In certain embodiments, the hair product restores damaged hair. In certain embodiments, the hair product is hair relaxant. In certain embodiments, the hair product adds natural shine. In certain embodiments, the hair product is a detangler. In certain embodiments, the hair product adds hair elasticity. In certain embodiments, the hair product moisturizes the hair. In certain embodiments, the thinning hair is cysteine-depleted. In certain embodiments, the present invention provides a method of treating, lessening the severity of, or delaying the onset of irritation and/or inflammation of the scalp in a subject in need thereof, comprising the step of administering to the subject a provided complex. In certain embodiments, the present invention provides a method of treating, lessening the severity of, or delaying the onset of dandruff in a subject in need thereof, comprising the step of administering to the subject a provided complex.

Application of certain agents to hair such as, for example, hair relaxants, which commonly comprise basic agents (e.g., NaOH), can cause skin irritation (e.g., irritation and/or inflammation of the scalp). In some embodiments, one or more inventive complexes is/are administered together with such an agent (e.g., hair relaxant) to reduce skin irritation and/or inflammation.

### 6. Combination Therapy

In some embodiments of the present invention, one or more inventive complexes is/are administered in combination with one or more other therapeutically active agents. In some embodiments, active agents administered in combination are administered as part of a single composition; in some embodiments, active agents administered in combination are administered as separate compositions.

To give but a few examples, in some embodiments, different inventive complexes are administered in combination.

In some embodiments, one or more inventive complexes is/are administered together with one or more other anti-inflammatory agents. Representative such anti-inflammatory agents include, for example, NSAIDs such as acetominaphen (Tylenol), aspirin, celecoxib (Celebrex), diclofenac (Voltaren), diflunisal (Dolobid), etodolac (Lodine), ibuprofen (Motrin), indomethacin (Indocin), ketoprofen (Orudis), ketorolac (Toradol), nabumetone (Relafen), naproxen (Aleve, Naprosyn), oxaprozin (Daypro), piroxicam (Feldene), salsalate (Amigesic), sulindac (Clinoril), tolmetin (Tolectin); and/or steroids such as glucocorticoids, cortisol, testoterone, estrogen, estradiol, progesterone, etc.

In some embodiments, one or more inventive complexes is/are administered together with one or more pain-relieving agents. Representative such pain relieving agents include, for example, NSAIDs such as acetominaphen (Tylenol), aspirin, celecoxib (Celebrex), diclofenac (Voltaren), diflunisal (Dolobid), etodolac (Lodine), ibuprofen (Motrin), indomethacin (Indocin), ketoprofen (Orudis), ketorolac (Toradol), nabumetone (Relafen), naproxen (Aleve, Naprosyn), oxaprozin (Daypro), piroxicam (Feldene), salsalate (Amigesic), sulindac (Clinoril), tolmetin (Tolectin); and/or steroids such as glucocorticoids, cortisol, testoterone, estrogen, estradiol, progesterone, etc. Alternatively or additionally, representative pain-relieving agents include, for example, articaine, benzocaine, bupivacaine, carticaine, chloroprocaine, cinchocaine/dibucaine, cocaine, cyclomethycaine, dimethyocaine/larocaine, etidocaine, levobupivacaine, lidocaine/lignocaine, mepvacaine, piperocaine, prilocaine, propoxycaine, procaine/novocaine, proparacaine, ropivacaine, saxitoxin, tetracaine/amethocaine, trimecaine, and/or combinations thereof.

In some embodiments, inventive complexes are administered together with glucocortocoids, aspirin, diclofenac, lidocaine, etc., and/or combinations thereof.

### Examples

Complexes of AFC compounds and binding partners as provided by the present invention may be prepared by any method known if the art. Non-limiting examples for preparing inventive complexes are illustrated below.

AFC compounds utilized in preparation of inventive complexes may be prepared or synthesized according to methods known in the art. To give but some examples, AFC compounds may be prepared or synthesized by methods disclosed in one or more of U.S. Pat. No. 5,043,268; U.S. Pat. No. 5,202,456; U.S. Provisional Application 61/007,234, filed Dec. 10, 2007; U.S. Provisional Application 61/065,939, filed Feb. 14, 2008; U.S. Provisional Application 61/113,498, filed Nov. 11, 2008; U.S. Publication 2009/0170917, published on Jul. 2, 2009; U.S. Provisional Application 61/066,075, filed on Feb. 15, 2008; U.S. application Ser. No. 12/616,781, filed Nov. 12, 2009; US Patent Publication Number 2009/0192332, published on Jul. 30, 2009, or PCT Publication Number WO2009/102997, published on Aug. 20, 2009; each of which is incorporated herein by reference in its entirety.

### Example 1

### Preparation of a Strontium Complex

Synthesis of Strontium (R)-2-acetamido-3-((2E,6E)-3,7,11-trimethyldodeca-2,6,10-trienylthio)propanoate (“AFC-strontium complex”): AFC (1 mmol, 0.367 g) was mixed with NaOH (1.1 mmol, 0.044 g) in water (10 ml, distilled water) while stirring. Upon formation of homogeneous solution SrCl₂ (1.1 mmol, 0.292 g) was added and the desired product crashed out as an off white solid (with a cottage cheese-like consistency). The reaction mixture was stirred for an additional 30 minutes. The desired product was then isolated by filtration and washed with water (4×10 ml) while agitating with spatula. Finally, the strontium complex was re-dissolved in THF (10 ml), filtered, concentrated and dried in high vacuum to yield pure (>98% by HPLC, 6.67 min, FASTGRAD method) product (0.471 g, 57% yield) as a beige glassy solid. CHN analysis of the strontium complex was consistent with 4 AFC : 1 Sr. ¹H NMR (500 MHz, CDCl₃): δ 1.52-1.71 (m, 12H), 1.86-1.97 (m, 11H), 2.91 (br s, 2H), 3.07 (br s, 2H), 4.51 (br s, 1H), 5.03 (br s, 2H), 5.11 (br s, 1H); ¹³C NMR (125 MHz, CDCl₃): δ 15.8 16.3, 16.5, 25.4, 25.8, 26.1, 27.7, 30.2 (br s), 40.7, 65.2, 118.1, 120.3, 123.1, 131.4, 132.3, 140.6, 149.2 (br s), 171.3, 174.2. SrNO₂ may be used in the above procedure in the place of SrCl₂ if desired.

### Example 2

### Preparation of Calcium and Magnesium Complexes

Calcium and magnesium complexes of AFC (“AFC-calcium complex” and “AFC-magnesium complex”) were prepared in a substantially similar manner as the AFC-strontium complex described in Example 1, except that instead of SrCl₂, CaCl₂, and MgCl₂ were used as starting materials.

### Example 3

### Preparation of a Strontium Complex

Synthesis of Strontium (R)-2-amino-3-((2E,6E)-3,7,11-trimethyldodeca-2,6,10-trienylthio)propanoate (“Farnesyl cysteine-strontium complex”): S-Farnesyl-L-cysteine (1 mmol, 0.325 g) was mixed with NaOH (1.1 mmol, 0.044 g) in water (10 ml, distilled water) while stirring. Upon formation of homogeneous solution SrCl₂ (1.1 mmol, 0.292 g) was added and the desired product crashed out as an off white solid (with a cottage cheese-like consistency). The reaction mixture was stirred for an additional 30 minutes. The desired product was then isolated by filtration and washed with water (4×10 ml) while agitating with spatula. Finally, the strontium complex was re-dissolved in THF (10 ml), filtered, concentrated and dried in high vacuum to yield pure (>96% by HPLC, 2.51 min, FASTGRAD method) product (0.492 g, 61% yield) as off-white amorphous solid.

### Example 4

### Preparation of a Glucosamine Complex

Synthesis of Glucosamine (R)-2-acetamido-3-((2E,6E)-3,7,11-trimethyldodeca-2,6,10-trienylthio)propanoate (“AFC-glucosamine complex”): AFC (1 mmol, 0.367 g) was mixed with NaOH (1.1 mmol, 0.044 g) in water/ethanol mixture (10 ml, 1:1 ratio) while stirring. Upon formation of homogeneous solution, glucosamine hydrochloride (1.1 mmol, 0.236 g) was added and the reaction mixture was stirred for about 30 minutes. Next, the mixture was concentrated to dryness and reconstituted in ethanol (20 ml). The organic solution was filtered, concentrated and dried in high vacuum to yield pure (>98% by HPLC, 6.67 min, FASTGRAD method) product (0.403 g, 76% yield) as a beige glassy solid.

### Example 5

### Preparation of a Nicotinamide Complex

Synthesis of Nicotinamide (R)-2-acetamido-3-((2E,6E)-3,7,11-trimethyldodeca-2,6,10-trienylthio)propanoate (“AFC-nicotinamide complex”): N-Acetyl-S-trans,trans-farnesyl-L-cysteine (367 mg, 1 mmol) was mixed with NaOH (40 mg in 10 mL of water, 1 mmol) and concentrated to dryness. The residue was dissolved in ethanol (10 ml) and nicotinamide monochloride (158.3 mg, 1 mmol) was added. The reaction mixture was additionally stirred for 1 hr. The resulting solution was filtered and concentrated. The desired product was crystallized from ethanol/acetonitrile as glassy solid (170 mg, 35% yield). ¹H NMR (500 MHz, CDCl₃): δ 1.52 (s, 6H), 1.54 (s, 3H), 1.58 (s, 3H), 1.84-2.07 (m, 8H), 2.76 (dd, J=12.1 Hz, J=14.2 Hz, 1H), 2.93 (s, J=7.4 Hz, J=14.2 Hz, 1H), 3.14 (d, J=12.1 Hz, 2H), 4.32 (t, J=4.5 Hz, 1H), 4.51 (br.s, 2H), 4.59 (t, J=7.5 Hz, 1H), 4.98-5.00 (m, 2H), 5.15 (t, J=12.1 Hz, 1H), 7.56 (s, 1H), 8.21 (s, 1H), 8.69 (s, 1H), 8.89 (s, 1H); ¹³C NMR (125 MHz, CDCl₃): δ 16.3, 16.4, 23.3, 25.9, 26.0, 27.7, 27.8, 30.4, 40.7, 40.9, 53.9, 117.1, 117.8, 118.3, 121.4, 125.1, 125.5, 126.9, 129.3, 131.9, 132.1, 134.4, 136.2, 140.6, 150.3, 150.4, 154.5, 168.2, 171.6, 175.9.

### Example 6

### Preparation of a Nicotinamide Complex

Synthesis of Nicotinamide (E)-4-((R)-1-carboxy-2-((2E,6E)-3,7,11-trimethyldodeca-2,6,10-trienylthio)ethylamino)-4-oxobut-2-enoate (“MFC-nicotinamide complex”): N-fumaryl-S-trans,trans-farnesyl-L-cysteine (423 mg, 1 mmol) was mixed with NaOH (80 mg in 10 mL of water, 2 mmol) and concentrated to dryness. The residue was dissolved in ethanol (10 ml) and nicotinamide monochloride (316.6 mg, 2 mmol) was added and the reaction mixture was additionally stirred for 1 hr. The resulting solution was filtered and concentrated. The desired product was crystallized from ethanol/acetonitrile as an off-white solid (301 mg, 45% yield). ¹H NMR (500 MHz, CDCl₃): δ 1.52 (s, 6H), 1.54 (s, 3H), 1.58 (s, 3H), 1.84-2.07 (m, 5H), 2.76 (dd, J=12.1 Hz, J=14.2 Hz, 1H), 2.93 (s, J=7.4 Hz, J=14.2 Hz , 1H), 3.14 (d, J=12.1 Hz, 2H), 4.32 (t, J=4.5 Hz, 1H), 4.51 (br.s, 2H), 4.59 (t, J=7.5 Hz, 1H), 4.98-5.00 (m, 2H), 5.15 (t, J=12.1 Hz, 1H) 7.35 (d, J=15.4 Hz, 1H), 6.69 (d, J=15.4 Hz, 1H), 7.56 (d, J=7.2 Hz, 2H), 8.22 (d, J=7.2 Hz, 2H), 8.68 (d, J=7.2 Hz, 2H), 8.89 (s, 2H); ¹³C NMR (125 MHz, CDCl₃): δ 16.3, 16.5, 25.3, 25.9, 27.7, 27.7, 31.2, 40.5, 40.9, 53.9, 117.2, 117.8, 118.4, 121.4, 125.1, 125.5, 126.9, 127.6, 129.3, 131.9, 132.1, 134.4, 136.2, 140.6, 150.3, 150.4, 154.5, 167.3, 168.2, 175.9.

### Example 7

### Preparation of a Calcium Complex

Synthesis of Calcium 4-((R)-1-carboxy-2-((2E,6E)-3,7,11-trimethyldodeca-2,6,10-trienylthio)ethylamino)-4-oxobutanoate (“SFC-calcium complex”): A dry 4-necked 10 L round bottom flask equipped with a mechanic stirrer and a reflux condenser was charged with t,t-farnesyl cysteine (991.8 g, 3.05 mol), succinic anhydride (366.5 g, 3.66 mol) and tetrahydrofuran under argon atmosphere. The mixture was vigorously stirred and under reflux condition for 1 hr. The progress of the reaction was analyzed by HPLC to ensure completion. The reaction mixture was cooled down to room temperature. The crude material was combined with NaOH and CaCl₂ solution as follows: to every 1 L of reaction solution, was added (1) 2 L deionized H₂O; (2) 500 mL 5 N NaOH; (3) 500 mL 6.8 N CaCl₂. The resulting as-formed precipitate was filtered and the collected solid was washed with deionized H₂O (3 L×5), and subsequently with acetone (4 L×1). The washed solid was then dried under high vacuum overnight to yield 2270 g of the product.

### Example 8

### Preparation of a Zinc Complex

Synthesis of Zinc (R)-2-acetamido-3-((2E,6E)-3,7,11-trimethyldodeca-2,6,10-trienylthio)propanoate (“AFC-zinc complex”): AFC (367 mg, 1 mmol) was mixed with NaOH (40 mg in 10 mL of water, 1 mmol). After stirring at ambient temperature for 10 min, ZnCl₂ (136.3 mg in 5 ml of water, 1 mmol) was added and the reaction mixture was additionally stirred for 1 hr. The resulting solid was then filtered and washed with water (3×10 ml), subsequently dissolved in ethanol (20 ml), filtered and concentrated to yield the desired product (267 mg, 67% yield) as off-yellow solid. ¹H NMR (500 MHz, CDCl₃): δ 1.51-1.72 (m, 12H), 1.87-1.99 (m, 11H), 2.89 (br s, 2H), 3.11 (br s, 2H), 4.54 (br s, 1H), 5.01 (br s, 2H), 5.14 (br s, 1H); ¹³C NMR (125 MHz, CDCl₃): δ 15.9 16.3, 16.4, 25.4, 25.9, 26.0, 27.7, 30.4 (br s), 40.7, 64.9, 119.1, 120.8, 123.3, 131.4, 134.7, 140.6, 149.4 (br s), 171.2, 175.2.

### Example 9

### Preparation of a Titanium Complex

Synthesis of Titanium (R)-2-acetamido-3-((2E,6E)-3,7,11-trimethyldodeca-2,6,10-trienylthio)propanoate (“AFC-titanium complex”): AFC (367 mg, 1 mmol) was mixed with NaOH (40 mg in 10 mL of water, 1 mmol) and concentrated to dryness. The residue was dissolved in tetrahydrofuran (10 ml) and after stirring at ambient temperature for 10 min, TiCl₄ (53.1 mg in 5 ml of water, 0.25 mmol) was added dropwise and the reaction mixture was additionally stirred for 1 hr. The resulting solution was filtered and concentrated to yield the desired product (344 mg, 91% yield) as off-yellow glassy solid.

### Example 10

### Preparation of a Silver Complex

Synthesis of Silver (R)-2-acetamido-3-((2E,6E)-3,7,11-trimethyldodeca-2,6,10-trienylthio)propanoate (“AFC-silver complex”): AFC (367 mg, 1 mmol) was mixed with NaOH (40 mg in 10 mL of water, 1 mmol). After stirring at ambient temperature for 10 min, AgNO₃ (169.9 mg in 5 ml of water, 1 mmol) was added and the reaction mixture was additionally stirred for 1 hr. The resulting solid was then filtered, washed with water (3×10 ml) and subsequently dissolved in ethanol (20 ml), filtered and concentrated to yield the desired product (397 mg, 84% yield) as yellowish glassy solid. ¹H NMR (500 MHz, CDCl₃): δ 1.52-1.64 (m, 12H), 1.91-2.07 (m, 11H), 3.14 (br s, 2H), 3.47-3.51 (br d, J=7.1 Hz, 2H), 4.75 (br s, 1H), 4.98-5.00 (m, 2H), 5.15 *br s, 1H), ¹³H NMR (125 MHz, DCl₃): δ 15.9, 16.1, 16.4, 25.3, 26.6, 26.8, 30.3, 34.2 (br s), 36.3 (br s), 40.0, 54.6 (br s), 118.0, 123.0, 123.7, 132.1, 136.3, 143.5 (br s), 170. (br s), 174.9 (br s).

### Example 11

### Preparation of a Strontium Complex

Synthesis of Strontium (E)-4-((R)-1-carboxy-2-((2E,6E)-3,7,11-trimethyldodeca-2,6,10-trienylthio)ethylamino)-4-oxobut-2-enoate (“MFC-strontium complex”): N-fumaryl-S-trans,trans-farnesyl-L-cysteine (423 mg, 1 mmol) was mixed with NaOH (80 mg in 10 mL of water, 2 mmol). After stirring at ambient temperature for 10 min, Sr(NO₃)₂ (211.6 mg in 10 ml of water, 1 mmol) was added and the reaction mixture was additionally stirred for 1 hr. The resulting solid was then filtered and washed with water (3×10 ml) and subsequently dissolved in ethanol (20 ml), filtered and concentrated to yield the desired product (347 mg, 78% yield) as white solid.

### Example 12

### Preparation of a Strontium Complex

Synthesis of Strontium 4-(R)-1-carboxy-2-((2E,6E)-3,7,11-trimethyldodeca-2,6,10-trienylthio)ethylamino)-4-oxobutanoate (“SFC-strontium complex”): N-succinyl-S-trans,trans-farnesyl-L-cysteine (423 mg, 1 mmol) was mixed with NaOH (80 mg in 10 mL of water, 2 mmol). After stirring at ambient temperature for 10 min Sr(NO₃)₂ (211.6 mg in 10 ml of water, 1 mmol) was added and the reaction mixture was additionally stirred for 1 hr. Then the resulting solid was filtered and washed with water (3×10 ml). The resulting solid was then dissolved in ethanol (20 ml), filtered and concentrated to yield the desired product (378 mg, 85% yield) as off-white solid.

### Example 13

Synthesis of Monosodium 4-(R)-1-carboxy-2-((2E,6E)-3,7,11-trimethyldodeca-2,6,10-trienylthio)ethylamino)-4-oxobutanoate (“SFC-monosodium”): A solution of sodium hydroxide (90 mg, 2.26 mmol) in DI H₂O (1 mL) was prepared first and cooled down to room temperature. Then, a 50 mL RB flask equipped with stir bar was charged a solution of SFC (960 mg, 2.26 mmol) in methanol (5 mL). The sodium hydroxide aqueous solution was added dropwise. The mixture was stirred at room temperature for 10 minutes and pH of the mixture was 6.65. The solvent was removed in vacuo and dried under high vacuum overnight to yield the desired product (749 mg, 74% yield). ¹H NMR (500 MHz, D₂O) δ 1.500 (s, 3H), 1.512 (s, 3H), 1.574 (s, 3H), 1.599 (s, 3H), 1.873-2.028 (m, 8H), 2.440-2.577 (m, 4H), 2.767 (dd, J=8.5, 14.0 Hz, 1H), 2.926 (dd, J=4.0, 14.0 Hz, 1H), 3.159 (d, J=7.5 Hz , 2H), 4.372 (dd, J=4.5, 8.0 Hz, 1H), 5.027-5.039 (m, 2H), 5.167 (t, J=7.5 Hz, 1H); ¹³C NMR (125 MHz, D₂O) δ 15.548, 15.579, 17.179, 25.172, 26.092, 26.334, 29.329, 30.901, 31.081, 33.149, 39.274, 39.338, 53.871, 119.513, 123.967, 124.400, 131.24, 135.166, 139.796, 174.147, 176.472, 178.645; ES-MS: mass calcd for Chemical Formula: C₂₂H₃₄NNaO₅S 447.56. Found (M+1) m/z 448.20.

### Example 14

Synthesis of Disodium 4-(R)-1-carboxy-2-((2E,6E)-3,7,11-trimethyldodeca-2,6,10-trienylthio)ethylamino)-4-oxobutanoate (“SFC-disodium”): A solution of sodium hydroxide (1.885 g, 47.126 mmol) in DI H₂O (5 mL) and methanol (20 mL) was prepared. Then, a 250 mL RB flask equipped with stir bar was charged a solution of SFC (10.0143 g, 23.563 mmol) in methanol (20 mL) and the sodium hydroxide solution. The mixture was stirred at room temperature for 10 minutes. The solvent was removed in vacuo and dried under high vacuum overnight to give the desired product (11.01 g, 99.6% yield). ¹H NMR (500 MHz, D₂O) δ 1.551 (s, 6H), 1.608 (s, 3H), 1.621 (s, 3H), 1.935-1.964 (m, 2H), 2.012-2.111 (m, 6H), 2.377-2.420 (m, 2H), 2.451-2.494 (m, 2H), 2.755 (dd, J=8.0, 13.5 Hz, 1H), 2.912 (dd, J=5.0, 14.0 Hz, 1H), 3.178 (d, J=8.0 Hz , 2H), 4.312 (dd, J=4.5, 7.5 Hz, 1H), 5.127 (dd, J=7.0, 13.5 Hz, 2H), 5.210 (t, J=7.5 Hz, 1H); ¹³C NMR (125 MHz, D₂O) δ 15.151, 15.268, 16.919, 24.811, 25.333, 25.680, 28.918, 32.247, 32.888, 38.704, 38.736, 54.319, 119.555, 124.071, 124.378, 133.246, 136.385, 140.445, 174.991, 177.066, 181.033; ES-MS: mass calcd for Chemical Formula: C₂₂H₃₃NNa₂O₅S 469.55 Found (M−Na+H+1) m/z 448.20; Anal. Calcd for C22H35NNa2O6S (SFC-disodium salt mono hydrate) C, 54.20; H, 7.24; N, 2.87; S, 6.58. Found: C, 54.51; H, 7.40; N, 2.84; S, 6.50.

### Example 15

### Synthesis of an Exemplary AFC Compound, N-acetyl-S-phytyl-L-cysteine

The present Example describes preparation of a particular exemplary AFC compound, N-acetyl-S-phytyl-L-cysteine, using a direct fusion synthetic method.

As used in this Example, the term “room temperature” or “RT” means a temperature between about 22° C. and about 27° C.

Proton Nuclear Magnetic Resonance (¹HNMR) spectroscopy was recorded on a Varian NMR 400 MHz spectrometer; dimethyl sulfoxide, chloroform (CDCl₃) was used as ¹HNMR solvent. The residual proton absorption of the deuterated solvent was used as the internal standard. All ¹HNMR chemical shift were reported as δ values in the parts per million (ppm).

HPLC analysis was performed using a phenomenex luna C₁₈(2)50×4.6 mm column. The mobile phase was 60% water, 40% acetonitrile containing 0.05% trifluoroacetic acid at 2 ml per minute flow rate for the first 2.5 minutes, followed by a gradient to 100% acetonitrile containing 0.05% TFA over 12 minutes. The eluent was observed at 214 nm.

Thin-layer chromatography (TLC) on silica gel plates is a useful method to monitor the progress of the reactions. Compounds with free amino groups can be detected with ninhydrin or ninhydrin-collidine reagents. An anisaldehyde-sulphuric acid reagent is useful for the detection of a wide variety of organic compounds, and makes it easy to monitor both reactants and products. Such TLC methods were used in the analysis of the following phytyl derivatives.

N-acetyl-L-cysteine (0.5 g, 3.06 mmol) and phytol (1.1 g, 3.7 mmol) were mixed in a 25 mL round bottomed flask. The resulting mixture was stirred at 120° C. After ten minutes, a catalytic amount of concentrated H₂SO₄ was added (about 200 μL). About 1 h after the addition of H₂SO₄, TLC/HPLC showed completion of the reaction. The reaction mixture was quenched with NaOH, washed with heptanes to remove the non-polar impurities. The aqueous phase was adjusted to a pH of 2 by addition of aqueous HCl and the product was extracted from ethyl acetate (3×10 mL). The combined organic phase (ethyl acetate) was washed with water, brine and dried over Na₂SO₄. The solvent was removed under reduced pressure to afford crude product, which was further purified by preparative HPLC. 630mg of pure product was collected as a semisolid with about a 48% yield. 1H-NMR (CDCl3): δ 6.45 (d, 1H), 5.20 (t, 1H), 4.75 (q, 1H), 3.22-3.14 (dd, 2H), 3.00-2.90 (dd, 2H), 2.05 (s, 3H), 1.62 (s, 3H), 1.40-1.00(m, 18H), 0.85 (s, 3H), 0.83 (s, 6H), 0.80 (s, 3H).

### Example 16

### Synthesis of Zinc complex of N-acetyl-S-phytyl-L-cysteine

N-acetyl-S-phytyl-L-cysteine (444 mg, 1 mmol) from Example 15 is mixed with NaOH (40 mg in 10 mL of water, 1 mmol). After stirring at ambient temperature for 10 min, ZnCl₂ (136.3 mg in 5 ml of water, 1 mmol) is added, and the reaction mixture is additionally stirred for 1 hr. Then the resulting solid is filtered and washed with water (3×10 ml) dissolved in ethanol (20 ml), filtered and concentrated to yield the desired product.

### Example 17

### Mouse Model of Inflammation-Edema, Erythema and MPO Background

The mouse ear model of contact irritation has been established as an appropriate model to determine whether topically applied anti-inflammatories inhibit the development of acute, chemically induced dermal irritation [see Van Arman, C. G. et al., Anti-inflammatory Drugs, Clin. Pharmacol. Ther. 16, 900-4 (1974); Young et al., Tachyphylaxis in 12-Otetradecanoylphorbolacetate- and Arachidonic Acid-Induced Ear Edema; J. Invest. Dermatol. 80:48-52, (1983); Tramposch et al., In Vivo Models of Inflammation, (Morgan D W, Marshall L A eds), Birkha\ddot{} user Verlag: Basel, pp 179-204, 1999; and Gordon et al., Topical N-Acetyl-S-Farnesyl-L-Cysteine Inhibits Mouse Skin Inflammation, and Unlike Dexamethasone, Its Effects Are Restricted to the Application Site, J. Invest. Dermatol., 128(3):643-54, 2008 March)]. Moreover, the mouse ear model has been used by various groups to identify and compare members of differing classes of anti-inflammatory agents with multiple mechanisms of action (reviewed in Tramposch et al., 1999, supra). The commonly used end points of inflammation are edema (Young et al., 1983, supra), (assayed by increase in ear thickness), neutrophil infiltration (which is measured by assaying for the neutrophil marker myeloperoxidase (“MPO”) (see Bradley et al., Cellular and Extracellular Myeloperoxidase in Pyogenic Inflammation, Blood, 60(3):618-22; 1982) and erythema (skin redness). Using this model, we investigated the in vivo anti-inflammatory activity of provided complexes to identify which structures possess physical or chemical properties critical for inhibiting innate inflammation in the skin.

### (a) Protocol-Edema Inhibition

The protocol for inducing in vivo acute contact inflammation on the ears of live mice has been described elsewhere (reviewed in Tramposch, 1999, supra). In brief, mice were sedated and their ears were treated with 1.2 μg/20 uL TPA (i.e., tetradecanoylphorbol-13-acetate). After 5 minutes, we dosed these TPA-treated ears with a single 8 μg/20 μL dose, a 2 μg/20 μL dose, or both doses, of provided complexes. After 24 hours, the mice were sacrificed and edema was measured by taking micrometer readings of each ear. The percent inhibition of edema was determined by taking the average ear thickness of compound-treated ears and dividing it by the average thickness of 12 ears that only received TPA and subtracting that value from 100%. These values were corrected for the thickness of normal, non TPA-treated mouse ears of littermate controls.

### (b) Protocol-Erythema Inhibition

Another well documented biomarker of skin inflammation is skin redness, termed erythema, which is caused by capillary congestion and dilation in response to various chemical and environmental insults (see Denig, N.I. et al., Irritant Contact Dermatitis. Clues to Causes, Clinical Characteristics, and Control, Postgrad Med., May (1998); 103(5):199-200, 207-8, 212-3). The protocol for measuring erythema inhibition by provided complexes was developed in-house by utilizing the CR-400 chroma meter from Konica Minolta (2). This instrument was used to measure the Δa*redness value from 6 mm biopsy punches taken 24 hours post TPA/compound treatment as described in the edema inhibition section above. The percent inhibition of erythema was determined by taking the average Δa*redness value of compound-treated ears and dividing it by the average Δa*value of 12 ears that only received TPA and subtracting that value from 100%. These values were corrected for the Δa* value of non TPA-treated mouse ears of littermate controls.

### (c) Protocol-MPO Inhibition

To assay for inhibition of dermal neutrophil infiltration by provided complexes, a standard method was used (see Bradley et al., 1982, supra; Young et al., 1983, supra; De Young et al, “Edema and Cell Infiltration in the Phorbol Ester-treated Mouse Ear are Temporally Separate and can be Differentially Modulated by Pharmacologic Agents”, Agents Actions, 26(3-4) : 335-41 (Mar 1989); and Rao et al. (1993) Comparative Evaluation of Arachidonic Acid (AA)- and Tetradecanoylphorbol Acetate (TPA)-Induced Dermal Inflammation, Inflammation 17:723-41). Briefly, we homogenized 6mm biopsy punches taken from both compound-treated ears as well as TPA-treated and non-treated control groups. We quantitated the levels of MPO by a colorimetric reaction that was measured spectrophotometrically. The percent inhibition of neutrophil infiltration by each complex was determined by comparing the average MPO levels in the presence and absence of these compounds. The calculation for percent inhibition of MPO was determined similar to that as described for calculating the percent edema inhibition, see the Edema Inhibition protocol, supra.

### Results

At the dose tested, the AFC-glucosamine complex showed relativity high activity for inhibiting edema and neutrophil infiltration, 70.84±5.25% and 73.15±6.0%, respectively. On the other hand, the AFC oil inhibited edema and neutrophil infiltration at lower levels than did the AFC-glucosamine complex (56.35% and 55.90%, respectively (n=1, data not shown)).

Surprisingly, the AFC-strontium complex had an ED₅₀ for edema inhibition of 21.7 mM as compared with 54.8 mM for the AFC oil. AFC-strontium complex also gave an almost 8-fold lower ED₅₀ value for inhibiting neutrophil infiltration than did the AFC oil, i.e., 5.02 mM for the strontium complex and 38.8 mM for the AFC oil.

### Example 18

### Biological Activity of Certain Complexes

The biological activity of certain inventive complexes as described herein was characterized using a variety of different assays as described herein. Tables 1a-c below summarize % inhibition of edema, MPO and erythema is the mouse ear inflammation assay (4% concentration), respectively. For Table 1a (edema), “+”indicates a range of 0-20%, “++” indicates a range of 20-50%, and “+++” indicates >50% inhibition of edema. For Table 1b (MPO), “+”indicates a range of 0-40%, “++” indicates a range of 40-80%, and “+++” indicates >80% inhibition of MPO. For Table 1c (erythema), “+”indicates a range of 0-20%, “++” indicates a range of 20-40%, and “+++” indicates >40% inhibition of erythema.

TABLE 1a

| | % Edema Inhibition |
|---|---|
| | in the Mouse Ear |
| | Inflammation Assay |
| Name | (4% concentration) |
| Compound of formula Ih (“AFC”) | +++ |
| Strontium chloride | + |
| Farnesyl Cysteine-Strontium complex | ++ |
| Farnesyl Cysteine-Calcium complex | + |
| Compound of formula Ih and | +++ |
| strontium (“AFC-strontium complex”) | |
| Compound of formula Ih and | +++ |
| glucosamine (“AFC-Glucosamine | |
| complex”) | |
| SFC | +++ |
| SFC-calcium complex | +++ |
| SFC-monosodium | ++ |
| SFC-disodium | ++ |

TABLE 1b

| | % MPO Inhibition |
|---|---|
| | in the Mouse Ear |
| | Inflammation Assay |
| Name | (4% concentration) |
| Compound of formula Ih (“AFC”) | ++ |
| Farnesyl Cysteine-Calcium complex | ++ |
| Compound of formula Ih and | +++ |
| strontium (“AFC-strontium complex”) | |
| Compound of formula Ih and | ++ |
| glucosamine (“AFC-Glucosamine | |
| complex”) | |
| SFC | +++ |
| SFC-calcium complex | ++ |
| SFC-monosodium | ++ |
| SFC-disodium | ++ |

TABLE 1c

| | % Erythema Inhibition |
|---|---|
| | in the Mouse Ear |
| | Inflammation Assay |
| Name | (4% concentration) |
| Compound of formula Ih (“AFC”) | ++ |
| Strontium chloride | + |
| Farnesyl Cysteine-Strontium complex | ++ |
| Compound of formula Ih and | +++ |
| strontium (“AFC-strontium complex”) | |
| SFC | + |
| SFC-calcium complex | +++ |
| SFC-monosodium | ++ |
| SFC-disodium | ++ |

As can be seen from the data above, the AFC-strontium complex consistently showed potency across the assays. The AFC-calcium complex showed comparable MPO inhibition to AFC compound alone, and was as effective at decreasing edema.

Assays were conducted at several concentrations to assay for dose-dependent inhibitory activity. Inhibitory activity curves and ED₅₀ values were then calculated. The AFC-strontium complex has an ED₅₀ of 315 μg/ear in the MPO assay, compared with an ED₅₀ of 651 μg/ear for AFC alone.

The findings described herein demonstrate, among other things, that certain complexes have superior activity, as measured by inhibition of MPO, than other complexes or uncomplexed AFC compounds. Ability to inhibit MPO reflects ability to inhibit dermal infiltration, such as neutrophil infiltration, among other things.

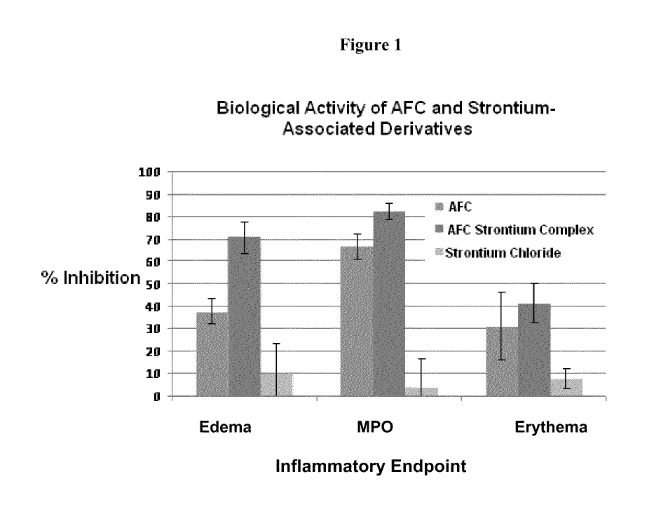

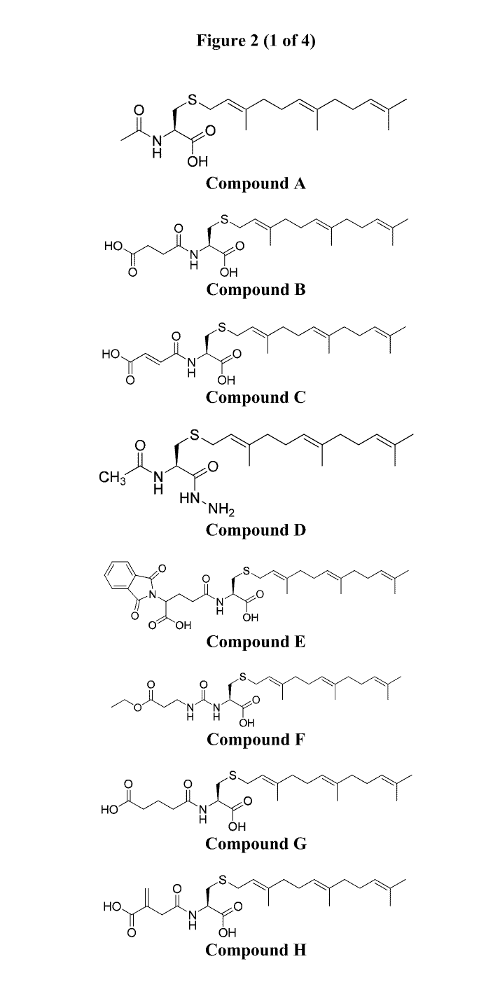

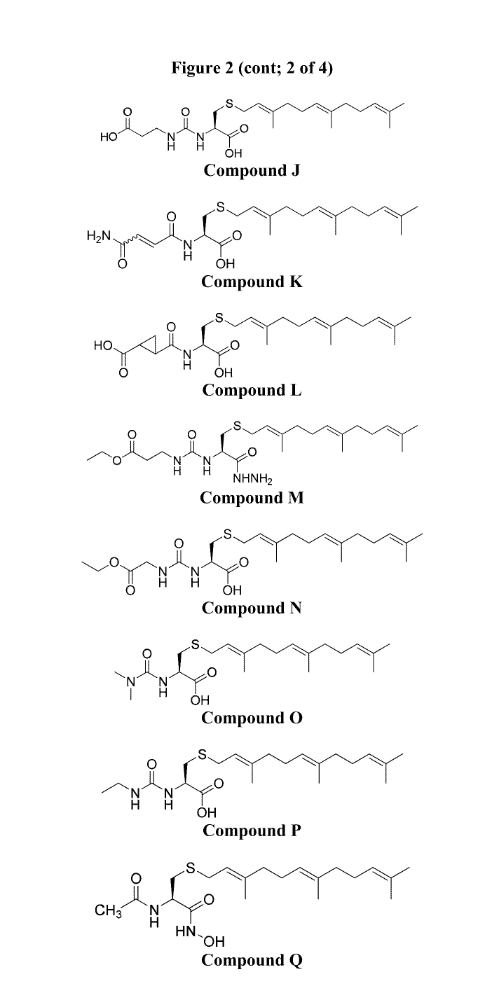

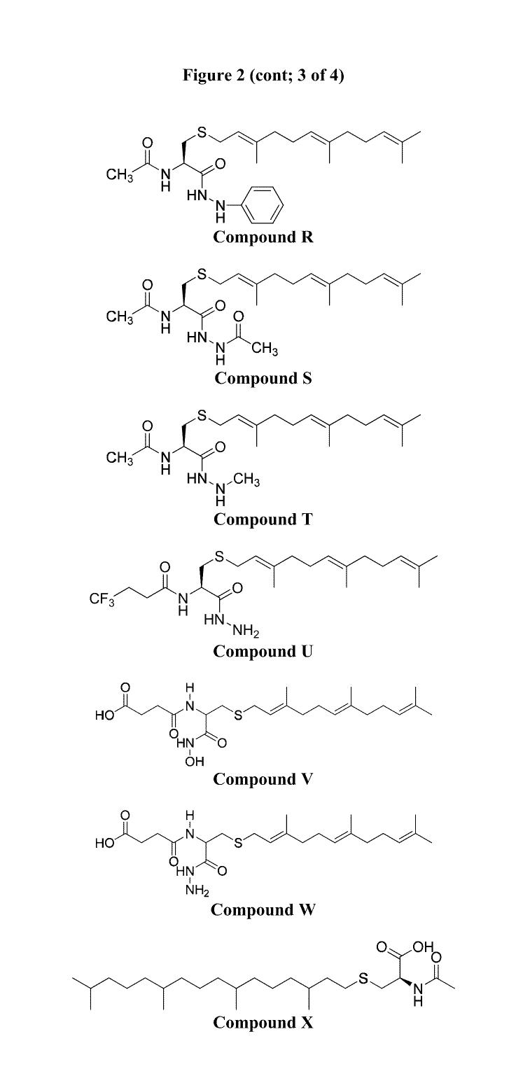

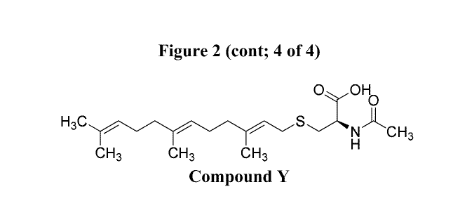

Furthermore, studies (see FIG. 1) have demonstrated that the increased activity observed with a strontium complex (as described herein) is not due solely to the strontium itself, as strontium chloride showed no significant activity.

### Example 19

### TPA-Induced Mouse Ear Model of Inflammation—Inhibition of Cytokine Levels

The protocol for inducing acute inflammation in mouse ears has been described elsewhere (reviewed in Tramposch, 1999, supra Skin Inflamation. In Vivo Models of Inflammation. D. W. Morgan and L. A. Marshall. Basel, Birkhäuser Verlag: 179-204). In brief, male Swiss Webster (ICR) mice 10-12 weeks age (Hilltop Lab Animals) are used for these experiments (6 animals per group). Mice receive 1.2 μg/20 μl TPA dissolved in acetone [10 μl applied both to the dorsal and ventral surfaces of the mouse ear (20 μl total) using a solvent pipette] to each ear to induce acute irritation. After 5 minutes, a sample of an AFC complex is applied at several concentrations in ethanol. After 24 hours treatment, mice are euthanized and 6-mm punch biopsy specimens are obtained from each ear, snap frozen in liquid nitrogen and stored in −80° C. until use. Ear biopsy specimens are homogenized with HTAB buffer using a Bio-Pulverizer (MP Biomedicals, 2×45 sec at 4 m/s). Samples are centrifuged at 10,000 rpm for 10 min at 4° C. Supernatants are subjected to cytokine profiling by ELISA for the stimulated production of cytokines using protein standards for quantification.

### Example 20

### LPS-TLR4-induced Inflammation Model in HMEC-1 Cells—Inhibition of Cytokine Levels

Human Microvascular Endothelial cells (HMECs) are cultured in EC basal medium (EBM; Cambrex, Walkersville, Md.), supplemented with 0.5% fetal bovine serum (FBS), epidermal growth factor (EGF) (10 ng/ml) hydrocortisone (1 μg/ml) and 100 U/ml penicillin/100 μg/ml streptomycin at 37° C. with 5% CO₂ (referred to as supplemented media). In order to avoid possible immunomodulating effects of these agents during agonist/antagonist treatments, for some periods, cells are kept in EBM supplemented only with 0.5% FBS and penicillin/streptomycin without EGF or hydrocortisone (referred to as depleted media). Cells are plated at a concentration of 0.25×10⁶ cells/well in supplemented media in 12-well plates. After cells are allowed to adhere (6-8 hours), media is changed to depleted media. After 24 hours, depleted media is removed and samples of fresh depleted media containing various concentrations of a provided complex in triplicate are added to the appropriate wells. Two hours later, to induce a pro-inflammatory response, LPS is added (100μM) in separate wells (in triplicate) (Bender et al. (2008) Exp. Dermatol. 17(9): 752-60; and Seiffert et al. (2006). J. Invest. Dermatol. 126(5): 1017-27). Cell cultures are examined for viability by Trypan blue exclusion and the reduction of 3-(4,5-dimethylthiazol-2-yl)-5-(3-carboxymethoxyphenyl)-2-(4-sulfophenyl)-2H-tetrazolium (MTS assay; Promega, Madison, Wis.) to determine the percentage of viable cells of various treatment concentrations of the AFC complex. After 6 hours of incubation, supernatants are harvested and assayed by enzyme-linked immunosorbent assays (ELISA) for the stimulated release of cytokine using appropriate protein standards (BD Pharmigen).

### Example 21

### ATPγS-Purinergic Receptor-Induced Inflammation Model in HMEC-1 Cells—Inhibition of Cytokine Levels

Human Microvascular Endothelial cells (HMECs) are cultured in EC basal medium (EBM; Cambrex, Walkersville, Md.), supplemented with 0.5% fetal bovine serum (FBS), epidermal growth factor (EGF) (10 ng/ml) hydrocortisone (1 μg/ml) and 100 U/ml penicillin/100 μg/ml streptomycin at 37° C. with 5% CO₂ (referred to as supplemented media). In order to avoid possible immunomodulating effects of these agents during agonist/antagonist treatments, for some periods, cells are kept in EBM supplemented only with 0.5% FBS and penicillin/streptomycin without EGF or hydrocortisone (referred to as depleted media). Cells are plated at a concentration of 0.25×10⁶ cells/well in supplemented media in 12-well plates. After cells are allowed to adhere (6-8 hours), media is changed to depleted media. After 24 hours, depleted media is removed and samples of fresh depleted media containing various concentrations of a provided complex, each in triplicate, are added to the appropriate wells. Two hours later, to induce a pro-inflammatory response, ATPγS was added (100 μM) in separate wells (in triplicate) (Bender et al. (2008) Exp. Dermatol. 17(9): 752-60; and Seiffert et al. (2006). J. Invest. Dermatol. 126(5): 1017-27). Cell cultures are examined for viability by Trypan blue exclusion and the reduction of 3-(4,5-dimethylthiazol-2-yl)-5-(3-carboxymethoxyphenyl)-2-(4-sulfophenyl)-2H-tetrazolium (MTS assay; Promega, Madison, Wis.) to determine the percentage of viable cells of various treatment concentrations of the AFC complex. After 6 hours of incubation, supernatants are harvested and assayed by enzyme-linked immunosorbent assays (ELISA) for the stimulated release of cytokines using appropriate protein standards (BD Pharmigen).

### Example 22

### TPA-Induced Inflammation Model in NHEK Cells—Inhibition of Cytokine levels

NHEK cells are cultured in keratinocyte growth medium (KGM; Gibco, Carlsbad, Calif.), in a serum-free environment, supplemented with EGF (10 ng/ml), hydrocortisone (1 μg/ml), bovine insulin (5 μg/ml) and human pituitary gland extract (2 mL) at 37° C. with 5% CO₂. To avoid any possible modulating effects of these agents during agonist/antagonist treatments, cells are kept in KGM supplemented without EGF or hydrocortisone (depleted medium). Cells are plated at a concentration of 0.25×10⁶ cells/ml in 12 well plates in supplemented media. After the cells are allowed to adhere (6-8 hours), media is changed to depleted media. After 24 hours, the depleted media is removed and samples of fresh depleted media containing various concentrations of a provided complex, each in triplicate, are added to appropriate wells. After 8 hours, the media is changed to media without the complex. After 16 hours, cell viability is determined by Trypan blue exclusion and MTS assay to determine the percent viability of various treatment concentrations of the complex. Cells are cultured in TPA (5 ng/ml) to induce a pro-inflammatory response and release of cytokines. After 5 hours of incubation, supernatants are harvested and assayed by ELISA for the stimulated release of cytokines. Various concentrations of the complex are added to tissue culture wells in triplicate two hours before addition of TPA as well as to cells not exposed to TPA. Cell viability is determined by Trypan blue exclusion and MTS assay, 16 hours after stimulation in a duplicate experiment, where cells are washed and fresh media added without TPA or a provided complex at the end of the stimulation period.

### Example 23

### TNFα-Induced Inflammation Model in HUVEC Cells—Inhibition of TNFα-Induced Cytokine Release

Human Umbilical Vein Endothelial (HUVEC) cells are cultured in endothelial growth medium-2 (EGM-2; Lonza; Walkersville, Md.), in a low serum environment (2% FBS), and supplemented with EGM-2 Bullet Kit (Lonza) at 37° C. with 5% CO₂. To avoid any possible modulating effects of these agents during agonist/antagonist treatments, cells are kept in EGM-2 supplemented without serum or growth factors (depleted medium). Cells are plated at a concentration of 1×10⁵ cells/ml in 96-well plates in supplemented media. After the cells are allowed to adhere (6-8 hours), media is changed to depleted media. Twenty-four hours later, media is removed and samples of fresh depleted media containing various concentrations of a provided complex in triplicate are added to appropriate wells. After 30 minutes of pre-incubation, cells are stimulated with recombinant Human TNF-α (1×10⁴ U/mL; Millipore, Billerica, Mass.) to induce a pro-inflammatory response and release of cytokines. After 4 hours of incubation, supernatants are harvested and assayed by ELISA for the stimulated release of cytokines. Cell viability is determined by Trypan blue exclusion and MTS assay to determine the percent viability of various treatment concentrations of the provided complex.

### Atopic Dermatitis

### Example 24

### Effects on Ovalbumin-Challenged Flaky Tail Mouse Model for Atopic Dermatitis

The flaky tail mouse strain carries a mutation in the gene for the epidermal protein filaggrin that is comparable for the mutation underlying human atopic dermatitis or eczema and is a model for the disease (Fallon et al. 2009. Nat. Genetics 41: 602-608). Topically challenging these mice with ovalbumin results in a atopic dermatitis like condition, exhibiting eczema and increased skin levels of TH2 and the cytokines IL4, IL5 and IL10, usually appearing 4-5 weeks following ovalbumin application. Using this model, we investigate the effectiveness of provided complexes in inhibiting and/or reducing the various end-points associated with atopic dermatitis. Exemplary end points include but are not limited to skin flakiness, skin levels of TH2 and other cytokines like IL4, IL5 and IL10. The protocol for cutaneous application of Ovalbumin to the intact skin of flaky tail mice has been described elsewhere (Fallon et al. 2009. Nat. Genetics 41: 602-608). In brief, the abdomens of 3-5 week ft/ft mice (6 animals per groups) are shaved 24 hours prior to cutaneous application and suspensions of Ovalbumin (50 μg in 50 μl PBS) are applied to the abdomen according to a strict regimen as described previously (Fallon et al. 2009. Nat. Genetics 41: 602-608). Two sets of experiments are conducted: in the first set, the mice are pretreated with a provided complex prior to and during the application of ovalbumin to study the effects of preventing and inhibiting the development of AD phenotype; and in the second set, the mice are treated with the complex following 4-5 weeks of ovalbumin treatment when the phenotype appears to study the effects of the compounds in treating the symptoms. For the complex tested, samples of the complex are applied at several concentrations in ethanol to study dose dependent effects. Following each experiment, mice are euthanized and 6-mm punch biopsy specimens from each abdomen are harvested, snap frozen in liquid nitrogen and stored in −80° C. until use. The abdominal skin specimens are homogenized with HTAB buffer using a Bio-Pulverizer (MP Biomedicals, 2×45 sec at 4 m/s). Samples are centrifuged at 10,000 rpm for 10 min at 4° C. Supernatants are subjected to cytokine profiling by ELISA for the levels of TH2, IL4, IL5, and IL10 using protein standards for quantification.

### K5.Stat3C Mouse Psoriasis Model

### Example 25

### K5.Stat3c Mouse Psoriasis Model—Inhibition of Helper-T-lymphocyte infiltration

Described below is an assay used to measure the biological activity of provided compounds, including the anti-psoriasis properties of the compounds, as measured by inhibition of T-helper lymphocyte infiltration determined using a psoriasis mouse model.

The spontaneous and injury induced appearance of plaques having the full psoriatic phenotype in a transgenic mouse constituitively expressing signal transducer and activator of transcription 3 (STAT3) under regulation of the keratin-5 promoter in basal epidermal keratinocytes (“K5.Stat3C mice”) has been recently reported (Sano et al. (2005). Nat Med 11(1): 43-9). In addition, the skin from these K5.Stat3C mice when allografted to immunodeficient nude mice do not develop plaques unless they are co-engrafted with activated T-cells, as occurs when human psoriatic skin when grafted to Severe Combined Immunodeficiency (SCID) mice (Wrone-Smith et al. 1996. J. Clin. Invest. 98: 1878-1887; Nickoloff et al. 1999. Am. J. Pathol. 155: 145-158), establishing the necessary interaction between the altered epidermis and the immune system. These CD3+ T-helper cells play a critical role in the psoriatic pathogenesis by controlling infiltration of T-lymphocytes. Thus, inhibiting CD3 cell expression is an attractive target for treating psoriasis. The protocol for studying the CD3+ Helper-T expression using the K5.Stat3C psoriasis mouse model has been previously described (Sano et al. (2005). Nat Med 11(1): 43-9). 5 mice per treatment group are used. Dorsal skin of 7-9 wk old BK5.STAT3C mice are shaved 48 hours prior to tape stripping. Mice are then anesthetized with Avertin and receive 30 strokes of tape stripping. Samples of a provided complex, dexamethasone (Dex, positive control) or acetone vehicle control are topically applied to the shaved area at indicated times and doses. Mice are injected with BrdU 30 minutes prior to sacrifice at day 5, and skin sections collected for histological assessment of dermal inflammatory infiltrates.

### Antiinflammatory and Antixoxidant Effects

### Example 26

### Inhibition of Oxidative Burst from Neutrophils

During inflammation, neutrophils produce and release superoxide ions, otherwise known as oxidative burst response, causing cytotoxicity. The present example demonstrates that certain AFC complexes show inhibition of oxidative burst response from neutrophils, thereby inihibiting neutrophil-mediated cytotoxic events. The superoxide release assay is based on published protocols (Goldstein et al. (1975). J. Clin. Invest. 56(5): 1155-63). Briefly, cells were pre-incubated for 10 min at 37° C. with a mixture of cytochrome c (75 μM final concentration), cytochalasin B (5 μg/ml) with or without superoxide dismutase (SOD) (10 μg/ml) and with or without AFC, AFC-Calcium complex, and AFC-Strontium complex (ranging from 0 to 100 μM). To initiate O₂⁻ release, fMLP (0.2 μM) was added the cells are incubated for 10 min at 37° C. Samples were then placed on ice for 5 min and subsequently centrifuged at 3,000 rpm at 4° C. The supernatant was then analyzed by spectrophotometric measurement at 550 and 556.5 nm. The ED₅₀ values for the reduction of superoxide formation, obtained with AFC, AFC-Calcium complex, and AFC-Strontium complex are depicted in Table 2.

TABLE 2

| Compound Name | ED50 Respiratory Burst (μM) |
|---|---|
| AFC | 43.26 |
| AFC-Calcium complex | 48.86 |
| AFC-Strontium complex | 35.60 |

### Antimicrobial Effects/Anti-Acne Effects

Described below are assays used to measure the biological activity of provided compounds, including the anti-bacterial properties of the compounds, as measured by inhibition of bacterial growth (Example 27) and determination of minimum bactericidal concentration (“MBC”) (Example 28).

### Example 27

### Inhibition of Propionibacterium Acnes Growth

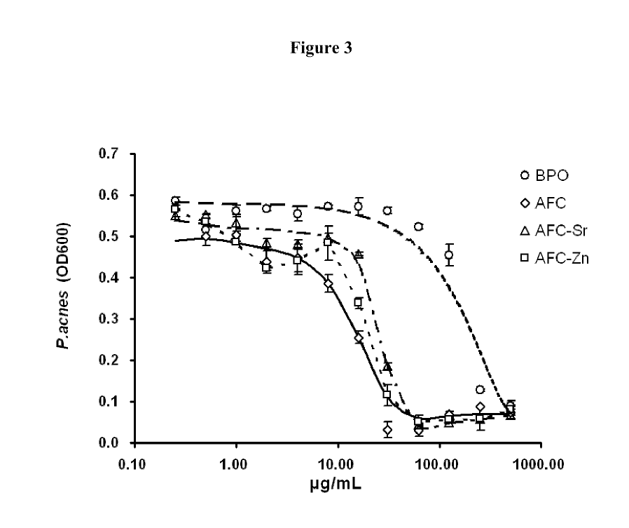

The present example demonstrates that certain AFC complexes of the present invention exhibit superior or similar anti-microbial activity when compared to benzoyl peroxide, a well-known anti-microbial and anti-acne agent. The assay for the inhibition of growth of Propionibacterium acnes bacteria is described elsewhere (Nakatsuji et al., J Invest Dermatol, 2009, 129: 2480-2488). In brief, the strain ATCC 6919 of P. acnes (American Type Culture Collection, Manassas, Va.) was cultured on Brucella agar (R01254, Remel, Lenexa, Kans.) supplemented with 5% (v/v) defibrinated sheep blood, vitamin K (5 mg/ml, Remel, Lenexa, Kans.), and hemin (50 mg/ml, Remel, Lenexa, Kans.), under an anaerobic condition using Gas-Pak (BD, Sparks, Md.) at 37° C. A single colony was inoculated in Reinforced Clostridium Medium (Oxford, Hampshire, England) and cultured at 37° C. under the anaerobic condition. Each of the inventive complexes—AFC-Sr, AFC-Ag and AFC-Zn as well as AFC were dissolved in 100% (v/v) DMSO. Samples of each inventive complex, AFC and a solution of benzoyl peroxide (“BPO”) were then each incubated with an inoculum of P. acnes at a concentration of 1×10⁶ CFU per mL in Reinforced Clostridium Medium in a 96-well microplate (100 μL per well) under anaerobic conditions for 72 hours. Samples of each were tested at final concentrations per well of 0.25 μg/mL, 0.5 μg/mL, 1.0 μg/mL, 1.95 μg/mL, 3.9 μg/mL, 7.8 μg/mL, 15.625 μg/mL, 31.25 μg/mL, 62.5 μg/mL, 125 μg/mL, 250μg/mL, and 500 μg/mL. A control well received only 5% (v /v) of DMSO in place of a sample of an inventive complex. After 72 hours incubation under anaerobic conditions, the P. acnes cultures in the 96-well microplate were mixed well and then absorbance readings at 600 nm were taken to determine bacterial growth. P. acnes growth curves was plotted and the concentration of each inventive AFC compound tested that yielded 50% inhibition of bacterial growth (IC50) was determined using SigmaPlot. IC50 results demonstrating the effect of inventive compounds on inhibition of P. acnes growth for benzoyl peroxide (“BPO”), AFC, AFC-Strontium complex, AFC-Silver complex and AFC-Zinc complex are shown in Table 3. Growth curves of P. acnes determined for BPO, AFC, AFC-Sr, AFC-Ag and AFC-Zn are depicted in FIG. 3.

TABLE 3

| Compound Name | IC50 (μg/mL) |
|---|---|
| BPO | 161 |
| AFC | 12.9 |
| AFC-Strontium complex | 12.6 |
| AFC-Zinc complex | 9.2 |
| AFC-Silver complex | 16.9 |

### Example 28

### Determination of Minimum Bactericidal Concentration

The present example demonstrates that certain AFC complexes of the present invention exhibit anti-microbial activity and exhibit low minimum bactericidal concentrations. The Minimal Bactericidal Concentration (“MBC”) of the inventive AFC complexes against P. acnes is determined using the following method. Sample solutions of AFC complexes, for example, AFC, AFC-Sr, AFC-Ag and AFC-Zn, dissolved in 100% (v/v) DMSO, are each incubated with an inoculum of P. acnes at a concentration of 1×10⁷ CFU/mL in a 96-well microplate with a total culture of 100 μl per well under anaerobic conditions, to yield final compound concentrations per well of 1 μg/mL, 5 μg/mL, 10 μg/mL, 25 μg/mL, 50 μg/mL, 100 μg/mL and 200 μg/mL. A control well receives only 5% (v /v) of DMSO in place of the test solution of inventive complex. Following 5 hours of incubation, the reaction mixture is serially (1:10-1:10⁶) diluted with PBS. The MBC is determined by inoculating the diluted culture (5 μl) onto a brucella agar plate (R01254, Remel, Lenexa, Kans.). 72 hours after inoculation, colonies on the plates are counted, CFUs (Colony Forming Units) are calculated and the data is plotted by using SigmaPlot.

### Example 29

### P. acnes-Induced Mouse Ear Model of Inflammation—MPO Endpoint

The methods for a model of P. acnes-inflammation are described elsewhere (Nakatsuji et al., J Invest Dermatol, 2008, 128: 2451-2457). Using this mouse in vivo model for P. acnes-inflammation, the present example further demonstrates that certain AFC complexes of the present invention, when topically applied to a site of inflammation induced by bacterial challenge, for example by P. acnes exhibit in vivo anti-inflammatory activity, as evidenced by the effect on the commonly-used inflammatory end-points such as neutrophil infiltration (MPO neutrophil marker), and are therefore useful as anti-acne agents.

The protocol for inducing inflammation using P. acnes bacterial challenge on the mouse ear was slightly modified from the method described previously (Natatsuji et al., 2008). Briefly, Swiss Webster (ICR, 6-8 weeks old) mouse ears were injected with living P. acnes culture intradermally. An amount of 20 μl aliquots of living P. acnes (ATCC 6919, 3×10⁶ CFU) suspended in PBS was intradermally injected in the central portion of the ear. As a control, 20 μl of PBS was injected into control animals. Significant cutaneous erythema, ear swelling (edema), and granulomatous response (MPO activity) were observed in P. acnes-injected ear 24 hours after the bacterial injection, but not induced by phosphate-buffered saline (PBS) injection.

To assay for inhibition of dermal neutrophil infiltration by AFC complexes, a standard method was used (see Bradley et al., J Invest Dermatol, 1982,78: 206-209; Young et al., J Invest Dermatol, 1983, 80: 48-52; De Young et al, Agents Actions, 1989, 26: 335-41; and Rao et al., Inflammation, 1993, 17: 723-41). Briefly, 6mm biopsy punches taken from both compound-treated ears as well as non-treated control ears were homogenized in 400 μl of 0.5% hexadecyltrimethylammonium bromide in 50 mM potassium phosphate buffer (pH 6.0) using the Fast Prep 24 (MP Biomedicals, Solon, Ohio). Supernatants were assayed for MPO activity using a model EL 340 96-well plate reader (BioTek Instruments, Winooski, Vt.). The percent inhibition of neutrophil infiltration by each AFC complex was determined by comparing the average MPO levels in the presence and absence of these compounds. The percent inhibition of MPO was determined by taking the average MPO activity of compound-treated ears and dividing it by the average MPO activity of 12 ears that only received the P. acnes challenge and subtracting that value from 100%. These values were corrected for the MPO activity of normal, non P. acnes-treated mouse ears of littermate controls. Summary of MPO activity ranges determined from an MPO activity assay for dexamethasone (administered at a dose of 1.6 mg/20 μL), clobetasol (administered at a dose of 0.1 mg/20 μL), salicylic acid (administered at a dose of 0.4 mg/20 μL), AFC (administered at a dose of 0.4 mg/20 μL), AFC-Strontium complex (administered at a dose of 0.4 mg/20 μL) and SFC-disodium are presented in Table 4. “+” indicates a range of 0-20%, “++” indicates a range of 20-40%, and “+++” indicates >40% inhibition.

TABLE 4

| Compound Name | MPO % Inhibition |
|---|---|
| Dexamethasone | +++ |
| Clobetasol | +++ |
| Salicylic Acid | ++ |
| AFC | +++ |
| AFC-Strontium complex | +++ |
| SFC-Disodium | +++ |

### Example 30

### P. acnes-Induced Mouse Ear Model of Inflammation—Cytokine Release

The protocol for inducing acute inflammation in mouse ears using P. acnes has been described elsewhere (Nakatsuji et al., J Invest Dermatol, 2008, 128: 2451-2457) and similar to the protocol described in Example 25. Using this mouse in vivo model for contact irritation, the present example demonstrates that certain inventive complexes, when topically applied, exhibit in vivo anti-inflammatory activities at sites of inflammation induced by P. acnes, in part, by inhibiting the levels of pro-inflammatory cytokines, such as IL-6, TNF-α, IL-8 and IL-1β, resulting in the observed effects on the inflammatory end-point of neutrophil infiltration (MPO neutrophil marker), as demonstrated in Example 25. The present example therefore demonstrates that certain inventive complexes are useful for treating bacterial induced inflammation and are therefore useful as anti-acne agents.

The protocol for inducing inflammation using P. acnes bacterial challenge on the mouse ear is slightly modified from the method described previously (Natatsuji et al., 2008). Briefly, Swiss Webster (ICR, 6-8 weeks old) mouse ears are injected with living P. acnes (strain ATCC 6919) culture intradermally. An amount of 20 μl aliquots of living P. acnes (ATCC 6919, 3×10⁶ CFU) suspended in PBS is intradermally injected in the central portion of the ear. As a control, 20 μl of PBS is injected into control animals. Ear tissue biopsies (6 mm), taken from both compound-treated ears and non-treated control ears are obtained and homogenized using a Fast Prep 24 (MP Biomedicals, Solon, Ohio) for two cycles of 45 seconds with Lysing Matrix A in mammalian extraction buffer (Pierce) with protease inhibitors cocktail (Roche). Supernatants are assayed by enzyme-linked immunosorbent assays (ELISA) for the stimulated release of IL-6, TNF-α, IL-8, and IL-1β, using appropriate protein standards (BD Pharmigen).

### Sunblocking Effects/Photoprotection

### Example 31

### Inhibition of UVB-Induced Cytokine Release in Reconstituted Epidermis

Acute exposure of human skin to ultraviolet radiation in the 280-320 nm range (UVB radiation) results in acute inflammatory response, characterized by both erythema and edema (Terui et al., Acta Derm Venereol, 2001, 81: 8-13). The present example demonstrates that certain complexes of the present invention exhibit anti-inflammatory and sunblocking/photoprotection effects, as observed with reconstituted epidermal cultures when exposed to UVB radiation. In brief, fully three-dimensional reconstituted human epidermal cultures (cultured adult human keratinocytes on a collagen base) are grown on the air-liquid interface for 17 days in deWned growth medium. A provided complex is applied topically at a dose of 2 μl of 0.1% solution in ethanol. In parallel, ethanol vehicle and AFC (positive control) are applied onto the surface of the stratum corneum (0.5 cm²). Tissues are irradiated with UVB radiation at a dose of 300 mJ/cm² using Solar Simulator BIOSUM. UVB-irradiated and non-irradiated control tissues are stored in the dark at room temperature. At the end of the UV exposure, the tissues are incubated at 37° C. with 5% CO₂ for 6 hrs. After 6 hrs of culture, supernatants from UVB-irradiated and non-irradiated (control) cells are collected for cytokine/chemokine ELISA assays.

### Example 32

### UVB-SKH1Hairless Mouse Model—Inhibition of Cytokine Release

The present example demonstrates that certain complexes of the present invention exhibit anti-inflammatory and sunblocking/photoprotection effects in vivo, as observed by the effects on UVB-induced erythema using UVB-SKH1 hairless mouse model. The protocol for UVB exposure is described elsewhere (Ahsan et al., Photochem Photobiol, 2007, 83: 986-993). In brief, 6-8 week-old female SKH1 hairless mice from Charles River Laboratories (Wilmington, Mass.) are employed. For UVB exposure, a UVR generator lamp (providing 280-320 nm radiation) (Daavlin Compan, Bryan, Ohio) is used as previously described (Ahsan et al., 2007) and monitored by periodic calibrations using a digital light meter (International Light IL 1400, Daavlin Company). To determine the UVB dose at which neutrophil infiltration (as measured by MPO activity), sunburnt cell and edema reaches maximal activities, five groups of five animals each are irradiated with UVB at a dose of 500 mJ/cm² daily for 5 days. One group not receiving any treatment serves as the control group. The four remaining groups of irradiated mice are biopsied immediately after the first UVB dose, 24 hours after the first UVB exposure, 24 hours after a second UVB exposure and 24 hours after a third exposure, respectively. Prior to each UVB dose and biopsy sampling (4 mm diameter punch), skin fold thickness ix measured for UVB-induced edema induction analysis using a precision caliper. Two biopsy specimens are obtained from each animal. One biopsy specimen is assayed histologically for infiltrating cells and sunburn cells after staining with hematoxylin and eosin (H&E) or immunohistochemically staining Lys16 antibody (Millipore) specific for neutrophils and quantified by image analysis. The second biopsy specimen is used to measure cytokines (IL-6, IL-8, TNF-a and GM-CSF) in skin homogenates processed as described in Example 19. Histochemical staining for hydrogen peroxide is performed by incubating skin sections with 100 mM Tris-HCl buffer (pH 7.5), containing 1 mg/ml glucose and 1 mg/ml diaminobenzidine (DAB) for 5-6 hours at 37° C. Sections are washed in distilled water and counterstained with methyl green (2% for 60 min). For biochemical analysis of neutrophil infiltration, biopsies are assayed for MPO activity as described in Example 17.

For studying the effect of provided complexes on UVB-induced inflammation when topically applied, SKH-1 hairless mice are divided into six groups of five animals each. The first group does not receive any treatment, the second group receives a topical application of 100 μl ethanol vehicle. The third group receives an application of a provided complex (4% w/v in ethanol using 100 μl per dose) on their dorsal skin. The fourth group is irradiated with UVB at a dose of 500 mJ/cm² daily for 5 days. The fifth group receive a topical application of a provided complex (4% w/v in EtOH using 100 μl per dose) followed by UVB irradiation, 30 min following treatment with a provided complex (pre-treatment group). The sixth group receive UVB treatment first followed by treatment with a provided complex (4% w/v in EtOH using 100 μl per dose), 5 min following UVB treatment (post-treatment group). After treatments with the complex and UVB dosing, biopsy specimens are obtained and skin fold thickness is measured for UVB-induced edema, cytokines, MPO activity and histological analysis as described above.

### Example 33

### Inhibition of UVA-Induced DNA damage in NHEK Cells

Ultraviolet A radiation (UVA, 320-400 nm) accounts for more than 90% of solar radiation and induces genomic damage to skin cells. The present example demonstrates that certain provided complexes have a protective effect on UVA-induced DNA damage in NHEK cells and therefore considered to have anti-oxidant/sunblocking/photoprotection effects. NHEK cells are cultured and pretreated with different concentrations of a provided complex for 2 to 24 hrs. Untreated and pretreated cells are then embedded in low melting point agarose and immediately exposed to UVA radiation (1 to 6 J/cm²). UVA-induced DNA damages are then quantified using single cell gel electrophoresis (Comet assay) (Singh et al., Exp Cell Res, 1988, 175: 184-191; De Meo et al., Mut Res, 1991, 260: 295-306).

### Safety Evaluation

### Example 34

### Mouse wound Healing Model

The healing of wounded skin is mediated by epidermal reepithelialization that requires keratinocyte migration. Glucocorticoids are known to inhibit wound healing (Ehrlich et al., Proc Soc Exp Biol Med, 1971, 137: 936-938; Leibovich et al., Am J Pathol, 1975, 78, 71-100). The inventors have shown that repithelialization of clobetasol-treated wounds are significantly delayed while AFC has no effects on mouse skin reepithelialization. The present example demonstrates that certain provided complexes, like AFC, do not interfere with the healing of wounded skin. The methods for studying mouse skin reepithelialization using both partial and full thickness mouse wound healing models have been described elsewhere (Kim et al. Wound Repair Regen, 2001, 9: 386-390). In brief, for partial thickness wounds, a 3 mm-diameter circle of epidermis is removed from the ventral auricle. The resulting wound is cleansed and dressed with an occlusive polyurethane film. The mouse ears are excised on days 3, 5, 8, and 11 post-operation and fixed in 10% buffered formalin. Following day 8 post-operation, sampling of treated mice is performed to determine completion of healing. For full thickness wounds, mice heads are shaved and depilated prior to excising the skin. The cleaned and dressed wounds are harvested and fixed at days 3, 5, 8 and 10 and fixed in 10% buffered formalin.

### Example 35

Skin Thinning Effect—Histology for Dermal and Epidermal Thickness

Skin thinning is a major adverse effect of chronic topical glucocorticoid use (Schwartz et al., J Invest Dermatol, 1994, 102: 241-246). The present example demonstrates that AFC and certain provided complexes do not cause skin thinning. AFC, provided complexes, Dexamethasone and vehicle control samples at different concentrations are applied to the flank skin of hairless mice daily for 2 weeks at doses that produce maximal inhibition of MPO in the ear. At the conclusion of the 2-week period, skin fold thickness is determined using a spring-loaded caliper. Histological evaluation is made to assess epidermal and dermal thickness using image analysis. Collagen synthesis is assayed on tissue sections immunohistochemically using a monoclonal antibody against the N-terminal peptide of pro-collagen following methods described elsewhere (Griffiths et al., N Engl J Med, 1993, 329: 530-535).

### Joint Inflammation

### Example 36

### Lewis Rat Adjuvant-Induced Arthritis Model

The protocol for using the Lewis rat adjuvant-induced arthritis model are described elsewhere (Yamashita et al., J Immunol, 2002, 168: 450-457; Zhao et al., J Orthop Sci, 2000, 5:397; and Barbier et al., Ann Rheum Dis, 1986, 45:67). In brief, male Lewis rats are immunized with the adjuvant at the base of the tail on Day 0. On Day 7, 14, or 21 (or shorter or longer, depending on the study's goals) the rats' rear paw volume are measured macroscopically by blinded observers for edema. The paws are then amputated and x-rayed for bone/joint destruction via a grading system of 0-4 called the “radiological index”.

### Example 37

### Type II Collagen Induced Arthritis

The methods for using the Type II collagen induced arthritis model has been described elsewhere (Lubberts et al. J Clin Invest, 2000, 105: 1697; and Lubberts et al., J Immunol, 2003, 2003: 2655-2662). Briefly, male DBA-1/BOM mice are immunized in the tail with bovine type II collagen that has been prepared and emulsified with an adjuvant. After an incubation period, the paws of the mice are macroscopically inspected for edema and assigned a grade by independent, blinded observers. The whole joints are removed, fixed, embedded in paraffin, sectioned, and stained for inflammatory cell infiltration. Bone and joint destruction, a characteristic of collagen-induced arthritis, are measured as well.

### Irritable Bowel Disease Model

### Example 38

### Acetic Acid Induced Irritable Bowel Disease in Rats

The methods for using the acetic acid-induced IBD rat model has been described elsewhere (La et al., World J. Gastroenterol., 2005, 11: 237-241. In brief, Irritable Bowel Syndrome (IBS) is induced in male Sprague-Dawley rats by intracolonic instillation of 4% acetic acid solution. The diseased rats are allowed to recover for six days. On day seven, collection and measurement of inflammation is performed. Colonic segments are removed and put under tension to simulate motor activity of intestinal circular muscles. The effect of a provided complex on the colonic motor activity is quantified by measuring the mean intraluminal pressure at a given concentration. The mean intraluminal pressure is digitally calculated by dividing an integral value of pressure (area under the pressure trace) by the number of data points (tracing time).

### Induced Asthma Model

### Example 39

### Rat Lung Sensitization with OVA and Bortdella pertussis

The protocol for using the Bordatella-induced rat asthma model has been described in Ramos-Barbón et al., J Clin Invest, 2005, 115: 1580-1589. Briefly, inbred Brown Norway rats are sensitized with ovalbumin (OVA) adsorbed in aluminum hydroxide dissolved in PBS, and heat killed Bordatella pertussis. Then the rats undergo an airway challenge using 5% aerosolized OVA or BSA delivered through orotracheal intubation using a rodent pulmonary mechanics system. The lungs are then processed via 2 mM EDTA/PBS perfusion through the right ventricle; broncho-alveolar lavage (BAL) fluid samples are collected through tracheostomy; the lungs are fixed at 25 cm H₂O by tracheobronchial infusion of 4% paraformaldehyde/PBS or 10% formalin/PBS; and tissue sections are obtained in parahilar and mid-sagittal orientation.

### Equivalents

Those skilled in the art will recognize, or be able to ascertain using no more than routine experimentation, that while the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any targeting moiety, any disease, disorder, and/or condition, any method of administration, any therapeutic application, etc.) can be excluded from any one or more claims, for any reason, whether or not related to the existence of prior art.

Publications discussed above and throughout the text are provided solely for their disclosure prior to the filing date of the present application. Nothing herein is to be construed as an admission that the inventors are not entitled to antedate such disclosure by virtue of prior disclosure.

Thus, although the invention has been described in detail with particular reference to these preferred embodiments, other embodiments can achieve the same results. Variations and modifications of the present invention will be obvious to those skilled in the art and it is intended to cover all such modifications and equivalents. The entire disclosures of all references, applications, patents, and publications cited above and/or in the attachments, and of the corresponding application(s), are hereby incorporated by reference.

1. A complex comprising a compound of formula I: 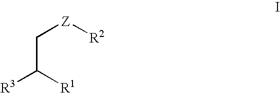
wherein:
R¹ is —C(O)X, wherein X is independently a protecting group, a halogen, R, —OR, —SR, —N(R)₂, a substituted or unsubstituted hydrazine, a substituted or unsubstituted 6-10 membered aryl ring, a substituted or unsubstituted 5-6 membered heteroaryl ring having 1-4 heteroatoms independently selected from nitrogen, oxygen, or sulfur; —NO₂; —PO₃H; —SO₃H;
—CN; substituted or unsubstituted heteroaryl; or one of the following moieties: 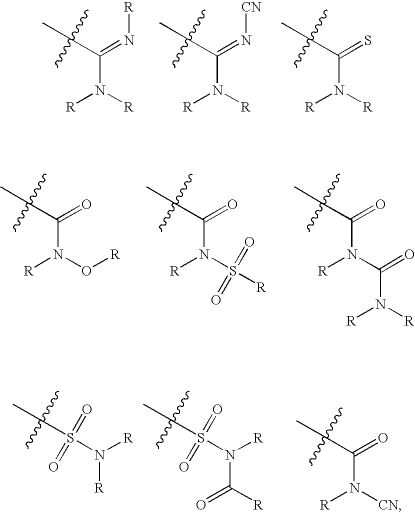
wherein each R is independently hydrogen or an optionally substituted group selected from C₁₋₆ aliphatic, C₁₋₆ heteroaliphatic, aryl, heteroaryl, or a cyclic radical;
R² is a substituted or unsubstituted, branched or unbranched C₁₀-C₂₅ aliphatic moiety;
R³ is —NH₂, a peptide, or —N(R⁴)(R⁵);
R⁴ is hydrogen or an optionally substituted group selected from C₁₋₆ aliphatic, C₁₋₆ heteroaliphatic, a cyclic radical, aryl or heteroaryl;
R₅ is heteroaryl; —C(═N—R⁶)(R⁷), wherein R⁶ is selected from hydrogen, aliphatic, and —N(R)₂, and R⁷ is selected from hydrogen, aliphatic, aryl, cyano, and —SO₂R; or C(O)LR⁸, wherein L is a covalent bond or a bivalent, branched or unbranched, saturated or unsaturated, C₂-C₆ hydrocarbon chain wherein one or more methylene units of L is independently replaced by —O—, —S—, —NH—, —C(O)—, —C(═CH₂)—, or C₃-C₆ cycloalkylene, wherein L is optionally substituted by one or more groups selected from halogen, phenyl, an 8-10 membered bicyclic aryl ring, a 5-6 membered heteroaryl ring having 1-4 heteroatoms independently selected from nitrogen, oxygen, or sulfur, an 8-10 membered bicyclic heteroaryl ring having 1-4 heteroatoms independently selected from nitrogen, oxygen, or sulfur, a 5- to 7-membered monocyclic having 1-2 heteroatoms independently selected from nitrogen, oxygen, or sulfur or a 7-10 membered bicyclic heterocyclyl ring having 1-2 heteroatoms independently selected from nitrogen, oxygen, or sulfur; and R₈ is —R, —OR, —N(R)₂, a cyclic radical, aryl, heteroaryl, wherein each R is independently hydrogen or an optionally substituted group selected from C₁₋₆ aliphatic, C₁₋₆ heteroaliphatic, aryl, heteroaryl, or a cyclic radical; or a substituted or unsubstituted peptidic moiety; and
Z is —S—, —O—, —NH—, —Se—, —S(═O)—, —S(═N)—, —Se(═O)—, or —SeO₂—; and
a binding partner non-covalently associated with the compound of formula I, and the amount of the binding partner is present in a ratio within the range of about 0.5:4.5 to 2:1 relative to the compound of formula I.

2. The complex according to claim 1 wherein the compound is of formula Ia: 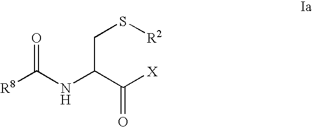
wherein
X is —OH, halogen, methyl, —SH, —NH₂, or —N(R)₂, wherein R is hydrogen or C₁₋₃ alkyl; and
R⁸ is C₁₋₃ alkyl.

3. The complex according to claim 1 wherein the compound is of formula Ib: 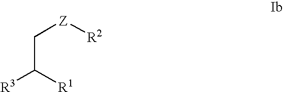
wherein
R¹ is —CO₂H, —CO₂R, —CONH₂, —NO₂, —PO₃H, —CN, or —SO₃H;
R² is farnesyl, phytyl, geranylgeranyl, substituted farnesyl, substituted phytyl, or substituted geranylgeranyl; and
R³ is —NH₂ or a peptide.

4. The complex according to claim 1 wherein the compound is of formula Ic: 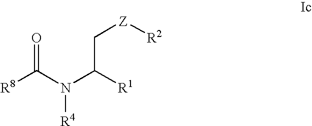
wherein
R¹ is substituted or unsubstituted heteroaryl, or one of the following moieties: 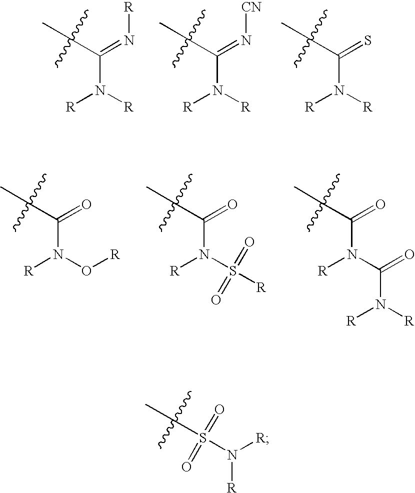
and
Z is —S—, —O—, —Se—, —SO—, —SO₂—, or —NH—.

5. The complex according to claim 1 wherein the compound is of formula Id: 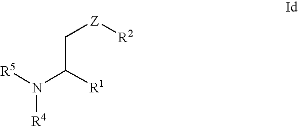
wherein
R¹ is substituted or unsubstituted heteroaryl, or one of the following moieties: 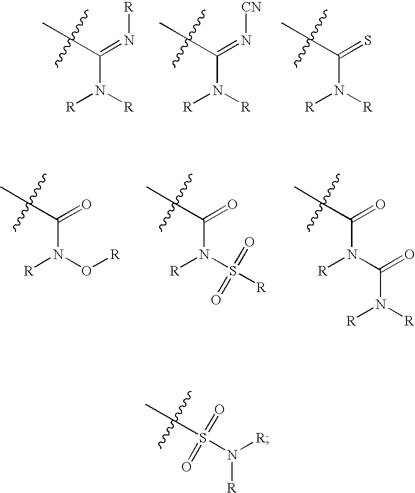
R⁵ is heteroaryl or —C(═NR⁶)(R⁷), where R⁶ and R⁷ are as described herein; and
Z is —S—, —O—, —Se—, —SO—, —SO₂—, or —NH—.

6. The complex according to claim 1 wherein the compound is of formula Ie: 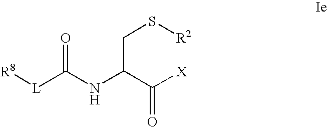
wherein
X is R, —OR, a hydrogen, aryloxy, amino, alkylamino, dialkylamino, heteroaryloxy, hydrazine, a 6-10 membered aryl ring, a 5-6 membered heteroaryl ring having 1-4 heteroatoms independently selected from nitrogen, oxygen, or sulfur, wherein each R is independently hydrogen or an optionally substituted group selected from C₁₋₆ aliphatic or C₁₋₆ heteroaliphatic;
L is a bivalent, branched or unbranched, saturated or unsaturated, C₂-C₆ hydrocarbon chain wherein one or more methylene units of L is independently replaced by —O—, —S—, —NH—, —C(O)—, —C(═CH₂)—, or C₃-C₆ cycloalkylene, wherein L is optionally substituted by one or more groups selected from halogen, phenyl, an 8-10 membered bicyclic aryl ring, a 5-6 membered heteroaryl ring having 1-4 heteroatoms independently selected from nitrogen, oxygen, or sulfur, an 8-10 membered bicyclic heteroaryl ring having 1-4 heteroatoms independently selected from nitrogen, oxygen, or sulfur, a 5- to 7-membered monocyclic having 1-2 heteroatoms independently selected from nitrogen, oxygen, or sulfur or a 7-10 membered bicyclic heterocyclyl ring having 1-2 heteroatoms independently selected from nitrogen, oxygen, or sulfur; and
R⁸ is hydrogen, —OH or —OR, wherein each R is independently hydrogen or an optionally substituted group selected from C₁ aliphatic or C₁₋₆ heteroaliphatic.

7. The complex according to claim 1 wherein the compound is of formula If: 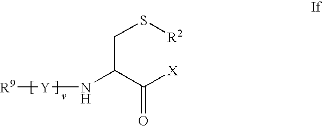
wherein
Y is a natural or unnatural amino acid;
v is an integer between 1 and 100, inclusive; and
R⁹ is hydrogen, a protecting group, or an optionally substituted group selected from C₁₋₆ aliphatic, C₁₋₆heteroaliphatic, aryl or heteroaryl.

8. The complex according to claim 1, wherein R¹ is a heteroaryl moiety of one of the formulae:

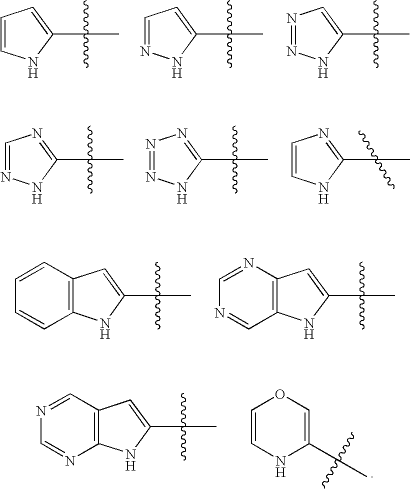

9. The complex according to claim 1, wherein R¹ is —CO₂H.

10. The complex according to claim 1, wherein R² is a farnesyl group.

11. The complex according to claim 1, wherein R³ is —NHCOCH₃.

12. The complex according to claim 1, wherein Z is —S—.

13. The complex according to claim 1, wherein the compound of formula I is N-acetyl-S-farnesyl-L-cysteine.

14. The complex according to claim 1 wherein the compound is of formula Ig: 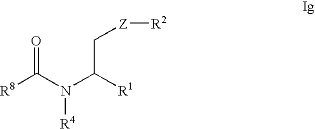
or a pharmaceutically acceptable salt, enantiomer, diastereomer, or double bond isomer thereof, wherein:
Z is —S—, —O—, Se—, —S(O)—, —SO₂—, or —NH—;
R¹ is a heteroaryl group, or a moiety selected from: 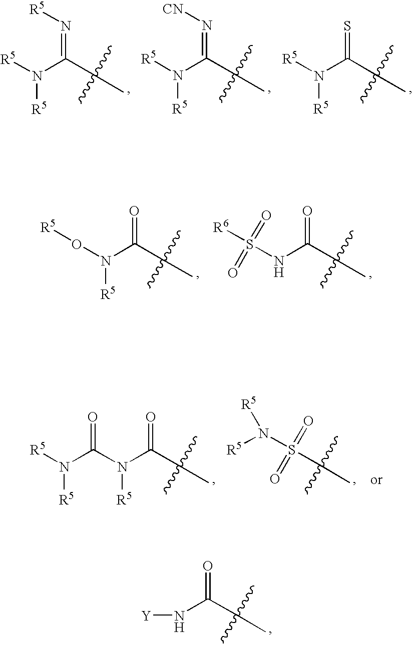
wherein at least one R⁵ group is H;
R⁵ is independently selected from H, alkyl, aryl, alkenyl, or alkynyl, wherein R⁵ is optionally substituted with one or two R⁷ groups;
R⁶ is H, alkyl, aryl, alkenyl, alkynyl, or a cyclic radical, where R⁶ is optionally substituted with one or two R⁷ groups;
Y is selected from H, —NH₂, —OH, —NH-phenyl, —NHC(O)CH₃, —NHCH₃, or —(C₁-C₈)alkyl;
R² is an aliphatic group substituted with one or more R⁷ groups;
R⁸ is alkoxy, aminoalkyl, alkyl, aryl, alkenyl, alkynyl, or a cyclic radical, where R⁸ is optionally substituted with one or two R⁷ groups;
R⁴ is H, alkyl, aryl, alkenyl, alkynyl, or a cyclic radical, where R⁴ is optionally substituted with one or two R⁷ groups; and
R⁷ is —NHC (═O)(C₁-C₈)alkyl, —(C₁-C₈)alkyl, —(C₁-C₈)alkenyl, —(C₁-C₈)alkynyl, phenyl —(C₂-C₅)heteroaryl, —(C₁-C₆)heterocycloalkyl, —(C₃-C₇)cycloalkyl, —O—(C₁-C₈)alkyl, —O—(C₁-C₈)alkenyl, —O—(C₁-C₈)alkynyl, —O-phenyl, —CN, —OH, oxo, halo, —C(═O)OH, —COhalo, —OC(═O)halo, —CF₃, N₃, NO₂, —NH₂, —NH((C₁-C₈)alkyl), —N((C₁-C₈)alkyl)₂, —NH(phenyl), —N(phenyl)₂, —C(═O)NH₂, —C(═O)NH((C₁-C₈)alkyl), —C(═O)N((C₁-C₈)alkyl)₂, —C(═O)NH(phenyl), —C(═O)N(phenyl)₂, —OC(═O)NH₂, —NHOH, —NOH((C₁-C₈)alkyl), —NOH(phenyl), —OC(═O)NH((C₁-C₈)alkyl), —OC(═O)N((C₁-C₈)alkyl)₂, —OC(═O)NH(phenyl), —OC(═O)N(phenyl)₂, —CHO, —CO((C₁-C₈)alkyl), —CO(phenyl), —C(═O)O((C₁-C₈)alkyl), —C(═O)O(phenyl), —OC(═O)((C₁-C₈)alkyl), —OC(═O)(phenyl), —OC(═O)O((C₁-C₈)alkyl), —OC(═O)O(phenyl), —S—(C₁-C₈)alkyl, —S—(C₁-C₈)alkenyl, —S—(C₁-C₈)alkynyl, and —S-phenyl, —NHS(O)₂-phenyl, —NHS(O)₂-alkyl, —NHS(O)₂—(C₁-C₈)alkenyl, —NHS(O)₂—(C₁-C₈)alkenyl, —SC(O)-phenyl, —SC(O)-alkyl, —SC(O)—(C₁-C₈)alkenyl, —SC(O)—(C₁-C₈)alkynyl, —O—S(═O)₂—(C₁-C₈)alkyl, —O—S(═O)₂—(C₁-C₈)alkenyl, —O—S(═O)₂—(C₁-C₈)alkynyl, —O—S(═O)₂—phenyl, —(CH₂)ₙNH₂, —(CH₂)ₙ—NH((C₁-C₈)alkyl), —(CH₂)ₙN((C₁-C₈)alkyl)₂, —(CH₂)ₙNH(phenyl), or —(CH₂)ₙN(phenyl)₂, wherein n is 1 to 8.

15. A complex comprising a compound of formula II: 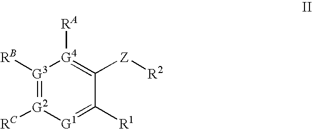
wherein each of G¹, G², G³, and G⁴ is N or CR^{D};
Z is S, O, Se, SO, SO₂, or NH;
R¹ is —C(O)X, wherein X is independently a protecting group, a halogen, R, —OR, —SR, —N(R)₂, a substituted or unsubstituted hydrazine, a substituted or unsubstituted 6-10 membered aryl ring, a substituted or unsubstituted 5-6 membered heteroaryl ring having 1-4 heteroatoms independently selected from nitrogen, oxygen, or sulfur; —NO₂; —PO₃H; —SO₃H; —CN; substituted or unsubstituted heteroaryl: or one of the following moieties: 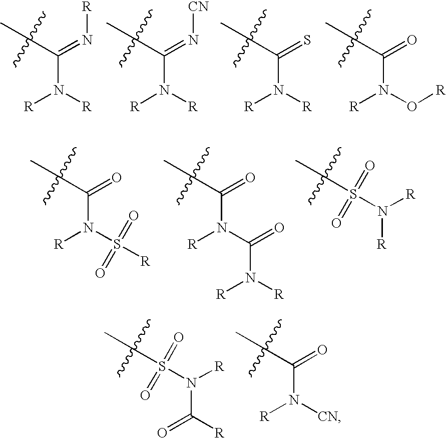
wherein each R is independently hydrogen or an optionally substituted group selected from C₁₋₆ aliphatic, C₁₋₆ heteroaliphatic, aryl, heteroaryl, or a cyclic radical;
R² is an optionally substituted aliphatic group;
R^{A}, R^{B}, R^{C}, and R^{D} are independently H, —NO₂, —OR¹⁰, halogen, alkylN(R¹⁰)₂, —N(R¹⁰)₂, —C(═O)R¹⁰, —C(═O)OR¹⁰, —S(R¹⁰), azido, —S—C≡N, alkyl, aryl, alkenyl, alkynyl, or a cyclic radical, wherein R^{A}, R^{B}, R^{C}, and R^{D} are further optionally substituted;
R¹⁰ is H, alkyl, aryl, alkenyl, alkynyl, or a cyclic radical, wherein R¹⁰ is further optionally substituted; and
a binding partner non-covalently associated with the compound of formula II, and the amount of the binding partner is present in a ratio within the range of about 0.5:4.5 to 2:1 relative to the compound of formula II.

16. A complex comprising a compound of formula III: 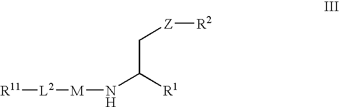
wherein:
L² is a bivalent, branched or unbranched, saturated or unsaturated, C₂-C₆ hydrocarbon chain wherein one or more methylene units of L is independently replaced by —O—, —S—, —N—, —C(O)—, —CF₂—, —C(═CH₂)—, —CH═CH—, or an optionally substituted arylene, heteroarylene, C₃-C₆ cycloalkylene, C₃-C₆ heterocycloalkylene, or an 8-10-membered bicyclic heterocyclic moiety,
and wherein L² is optionally substituted by one or more groups selected from halogen, C₁-C₆ alkyl, phenyl, biphenyl, -benzyl, —CH₂-phenol, —CH(phenyl)₂, —OH, —NH₂, —NHC(O)CH₃, —NHC(O)NHCH₂CH₃, —C(O)NH₂, —C(O)NHCH₂CH₃, —CH₂C(O)OCH₂phenyl, —(CH₂)₂SCH₃, —(CH₂)₂C(O)NH₂, —(CH₂)₂C(O)OH, an 8-10 membered bicyclic aryl ring, a 5-6 membered heteroaryl ring having 1-4 heteroatoms independently selected from nitrogen, oxygen, or sulfur, an 8-10 membered bicyclic heteroaryl ring having 1-4 heteroatoms independently selected from nitrogen, oxygen, or sulfur, a 5- to 7-membered monocyclic having 1-2 heteroatoms independently selected from nitrogen, oxygen, or sulfur or a 7-10 membered bicyclic heterocyclyl ring having 1-2 heteroatoms independently selected from nitrogen, oxygen, or sulfur;
M is —C(O)—, —C(S), or —SO₂—;
R¹¹ is hydrogen, F, CF₃, C₁-C₄ alkyl, —OH, —C(O)CH₃, —NH(OR¹²), —N(R¹²)₂, —NHN(R¹²)₂, —SO₂R¹², —NH-phenyl, —SO₂-phenyl, -phenyl-NO₂, or —OR¹², wherein each R¹² is independently hydrogen or an optionally substituted group selected from C₁₋₆ aliphatic or C₁-₆ heteroaliphatic;
R¹ is —C(O)X, wherein X is independently R¹², —C(O)NHNH₂, —OR¹², a hydrogen, aryloxy, amino, alkylamino, dialkylamino, heteroaryloxy, hydrazine, a 6-10 membered aryl ring, a 5-6 membered heteroaryl ring having 1-4 heteroatoms independently selected from nitrogen, oxygen, or sulfur; and
R² is a substituted or unsubstituted, branched or unbranched C₁₀-C₂₅ aliphatic moiety;
Z is —O—, —N—, —S—, —Se—, —S(O)—, —S(═N)—, —SO₂—, —Se(O)—, or —Se(O)₂—; and
a binding partner non-covalently associated with the compound of formula III, and the amount of the binding partner is present in a ratio within the range of about 0.5:4.5 to 2:1 relative to the compound of formula III.

17. A complex comprising a compound of formula IV: 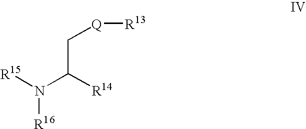
wherein:
R¹⁴ is an optionally substituted heteroaryl group or: 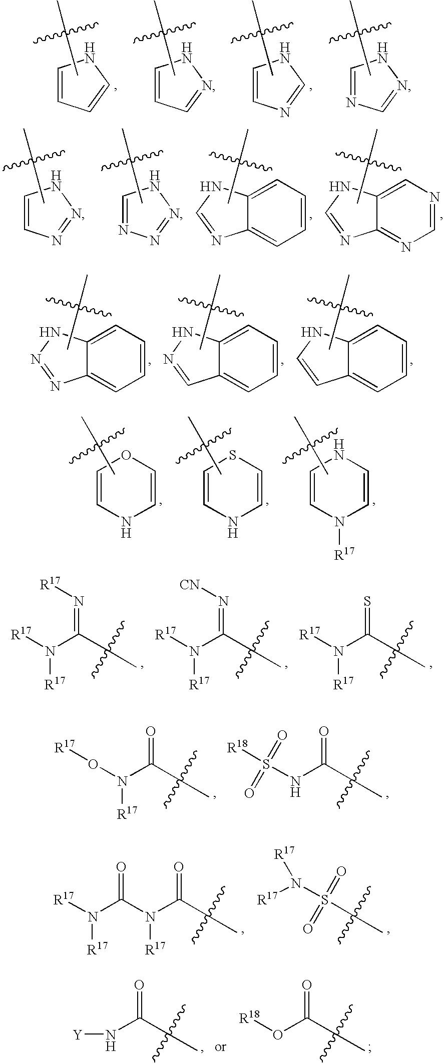
R¹³ is an aliphatic group substituted with one or more R¹⁹ groups;
R¹⁵ is an optionally substituted heteroaryl group, or a group selected from: 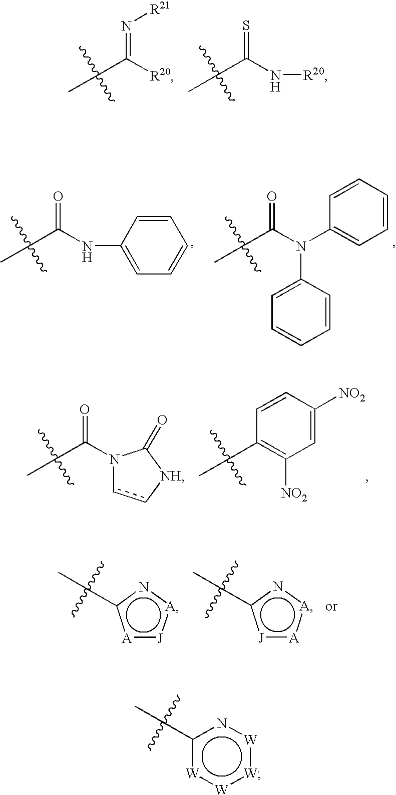
Y is selected from H, —NH₂, —OH, —NH-phenyl, —NHC(O)CH₃, —NHCH₃, or —(C₁-C₈)alkyl;
W is independently —C(R²²)— or N;
R²² is halo, hydrogen, CF₃, N(R¹⁷)₂, oxo, alkyl, alkenyl, alkynyl or aryl;
J is —O—, S, —N—, —N(R¹⁷)—, —C(R²³)— or —C(R¹⁸)—;
A is independently —C(R²³)—, —N— or —O—;
R²³ is hydrogen, F, CH₃, CF₃, OH, —NH₂, —NHNH₂, alkyl, alkenyl, alkynyl or aryl;
R¹⁶ is H, alkyl, aryl, alkenyl, alkynyl, or a cyclic radical, wherein R¹⁶ is optionally substituted with one or two R¹⁹ groups;
R¹⁷ is independently H, alkyl, aryl, alkenyl, or alkynyl, or —C(═O)O-butyl wherein R¹⁷ is optionally substituted with one or two R¹⁹ groups;
R¹⁸ is H, alkyl, aryl, alkenyl, alkynyl, or a cyclic radical, wherein R¹⁸ is optionally substituted with one or two R¹⁹ groups;
R¹⁹ is —NHC(═O)(C₁-C₈)alkyl, —(C₁-C₈)alkyl, —(C₁-C₈)alkenyl, —(C₁-C₈)alkynl, phenyl, —(C₂-C₅)heteroaryl, —(C₁-C₆)heterocycloalkyl, —(C₃-C₇)cycloalkyl, —O—(C₁-C₈)alkyl, —O—(C₁-C₈)alkenyl, —O—(C₁-C₈)alkynyl, —O-phenyl, —CN, —OH, oxo, halo, —C(═O)OH, 'COhalo, —OC(═O)halo, —CF₃, N₃, NO₂, —NH₂, —NH((C₁-C₈)alkyl), —N((C₁-C₈)alkyl)₂, —NH(phenyl), —N(phenyl)₂, —C(═ONH₂, —C(═O)NH((C₁-C₈)alkyl), —C(═O)N((C₁-C₈)alkyl)₂, —C(═O)NH(phenyl), —C(═O)N(phenyl)₂, —OC(═O)NH₂, —NHOH, —NOH((C₁-C₈)alkyl), —NOH(phenyl), —OC(═O)NH((C₁-C₈)alkyl), —OC(═O)N((C₁-C₈)alkyl)₂, —OC(═O)NH(phenyl), —OC(═O)N(phenyl)₂, —CHO, —CO((C₁-C₈)alkyl), —CO(phenyl), —C(═O)O((C₁-C₈)alkyl), —C(═O)O(phenyl), —OC(═O)((C₁-C₈)alkyl), —OC(═O)(phenyl), —OC(═O)O((C₁-C₈)alkyl), —OC(═O)O(phenyl), —S—(C₁-C₈)alkyl, —S—(C₁-C₈)alkenyl, —S—(C₁-C₈)alkynyl, and —S-phenyl, —NHS(O)₂-phenyl, —NHS(O)₂-alkyl, —NHS(O)₂—(C₁-C₈)alkenyl, —NHS(O)₂—(C₁-C₈)alkynyl, —NHS(O)₂, —SC(O)-phenyl, —SC(O)-alkyl, —SC(O)—(C₁-C₈)alkenyl, —SC(O)—(C₁-C₈)alkenyl, —SC(O)—(C₁-C₈ alkynyl), —O—(═O)₂—(C₁-C₈)alkyl, —O—S(═O)₂—(C₁-C₈)alkenyl, —O—S(═O)₂—(C₁-C₈)alkynyl, —O—S(═O)₂-phenyl, —(CH₂)ₙNH₂, —(CH₂)ₙ—NH((C₁-C₈)alkyl), —(CH₂)ₙN((C₁-C₈)alkyl)₂, —(CH₂)ₙNH(phenyl), or —(CH₂)ₙN(phenyl)₂, wherein n is 1 to 8;
R²⁰ is H, alkyl, alkenyl, alkynyl, aryl, —N(R¹⁷)₂;
R²¹ is H, alkyl, alkenyl, alkynyl, aryl, —CN, —S(═O)₂—R¹⁸ or —C(═O)O-t-butyl; and
Q is —S—, —O—, —Se—, —S(O)—, —SO₂—, or —NH—;
each of the dashed lines independently represents the presence or absence of a double bond; and
a binding partner non-covalently associated with the compound of formula IV, and the amount of the binding partner is present in a ratio within the range of about 0.5:4.5 to 2:1 relative to the compound of formula IV.

18. The complex according to claim 17, wherein R¹³ is

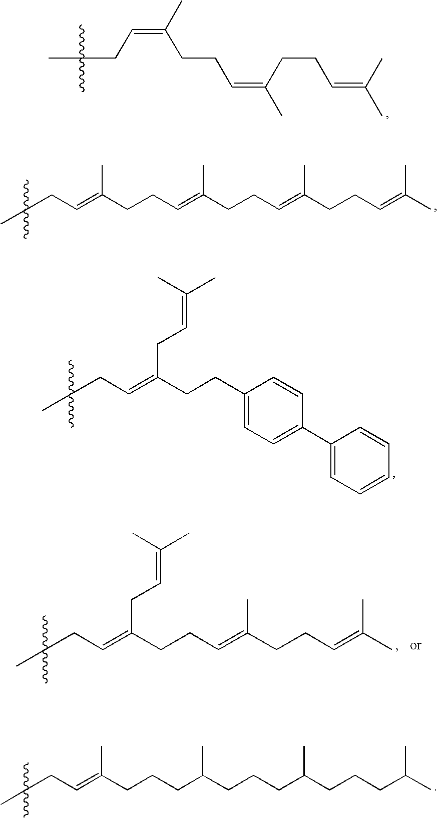

19. A complex comprising a compound of formula V: 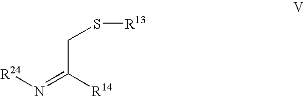
wherein:
R¹⁴ is an optionally substituted heteroaryl group or: 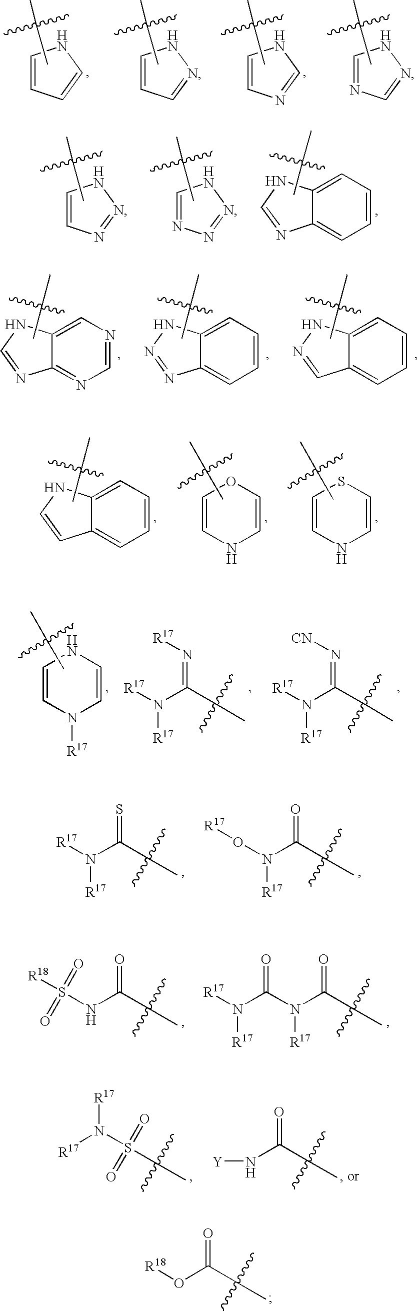
Y is selected from H, —NH₂, —OH, —NH-phenyl, —NHC(O)CH₃, —NHCH₃, or —(C₁-C₈)alkyl;
R¹³ is an aliphatic group substituted with one or more R¹⁹ groups;
R²⁴ is independently H, 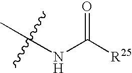
or —NH—S(O)₂R²⁵;
R²⁵ is independently H, (C₁-C₄)alkyl or aryl;
R¹⁷ is independently H, alkyl, aryl, alkenyl, or alkynyl, wherein R¹⁷ is optionally substituted with one or two R¹⁹ groups;
R¹⁸ is H, alkyl, aryl, alkenyl, alkynyl, or a cyclic radical, wherein R¹⁸ is optionally substituted with one or two R¹⁹ groups;
R¹⁹ is —NHC(═O)(C₁-C₈)alkyl, —(C₁-C₈)alkyl, —(C₁-C₈)alkenyl, —(C₁-C₈)alkynyl, phenyl —(C₂-C₅)heteroaryl, —(C₁-C₆)heterocycloalkyl, —(C₃-C₇)cycloalkyl, —O—(C₁-C₈)alkyl, —O—(C₁-C₈)alkenyl, —O—(C₁-C₈)alkynyl, —O-phenyl, —CN, —OH, oxo, halo, —C(═O)OH, —COhalo, —OC(═O)halo, —CF₃, N₃, NO₂, —NH₂, —NH((C₁-C₈)alkyl), —N((C₁-C₈)alkyl)₂, —NH(phenyl), —N(phenyl)₂, —C(═O)NH₂, —C(═O)NH((C₁-C₈)alkyl), —C(═O)N((C₁-C₁-C₈)alkyl)₂, —C(═O)NH(phenyl), —C(═O)N(phenyl)₂, —OC(═O)NH₂, —NHOH, —NOH((C₁-C₈)alkyl), —NOH(phenyl), —OC(═O)NH((C₁-C₈)alkyl), —OC(═O)N((C₁-C₈)alkyl)₂, —OC(═O)NH(phenyl), —OC(═O)N(phenyl)₂, —CHO, —CO((C₁-C₈)alkyl), —CO(phenyl), —C(═O)O((C₁-C₈)alkyl), —C(═O)O(phenyl), —OC(═O)((C₁-C₈)alkyl), —OC(═O)(phenyl), —OC(═O)O((C₁-C₈)alkyl), —OC(═O)O(phenyl), —S—(C₁-C₈)alkyl, —S—(C₁-C₈)alkenyl, —S—(C₁-C₈)alkynyl, and —S-phenyl, —NHS(O)₂-phenyl, —NHS(O)₂-alkyl, —NHS(O)₂-(C₁-C₈)alkenyl, —NHS(O)₂—(C₁-C₈)alkynyl, —NHS(O)₂, —SC(O)-phenyl, —SC(O)-alkyl, —SC(O)—(C₁-C₈)alkenyl, —SC(O)—(C₁-C₈ alkynyl) —O—S(═O)₂—(C₁-C₈)alkyl, —O—S(═O)₂—(C₁-C₈)alkenyl, —O—S(═O)₂—(C₁-C₈)alkynyl, —O—S(═O)₂-phenyl, —(CH₂)ₙNH₂, —(CH₂)ₙ—NH((C₁-C₈)alkyl), —(CH₂)ₙN((C₁-C₈)alkyl)₂, —(CH₂)ₙNH(phenyl), or —(CH₂)ₙN(phenyl)₂, wherein n is 1 to 8; and
a binding partner non-covalently associated with the compound of formula V, and the amount of the binding partner is present in a ratio within the range of about 0.5:4.5 to 2:1 relative to the compound of formula V.

20. A composition comprising a complex of claim 2.

21. A pharmaceutical composition comprising a complex of claim 2; and at least one pharmaceutically acceptable carrier or excipient.

22. The pharmaceutical composition of claim 21, which composition is formulated for oral delivery.

23. The pharmaceutical composition of claim 21, which composition is formulated for buccal delivery.

24. The pharmaceutical composition of claim 21, which composition is formulated for topical administration.

25. The pharmaceutical composition of claim 21, which composition is formulated for administration by inhalation.

26. The pharmaceutical composition of claim 21, which composition is formulated for parenteral administration.

27. A method of treating, lessening the severity of, or delaying onset of an inflammatory disease or disorder, comprising administering an amount of a complex according to claim 2 to a subject.

28. The method according to claim 27 wherein the inflammatory disease is selected from the group consisting of irritant contact dermatitis, atopic dermatitis, sebhorric dermatitis, psoriasis, contact allergy, photosensitivity, contact urticaria, rosacea, skin abrasion, bone and/or joint inflammation,

29. A method for inhibiting or reducing sensory irritation, erythema, edema or vesiculation comprising administering to a subject a complex according to claim 2.

30. The method according to claim 29 wherein the sensory irritation is selected from the group consisting of a sting, burn or itch.

31. A method of treating, lessening the severity of, or delaying onset of an epithelial condition, caused or aggravated by bacteria, comprising administering a complex according to claim 2 to a subject in need thereof

32. The method according to claim 31 wherein the epithelial condition is selected from a skin condition (such as cellulitis; erysipelas; impetigo; ecthyma e.g. ecthyma gangrenosum; cutaneous anthrax; necroticizing fasciitis; toe web infections; sycosis barbae; furuncles and carbuncles; Staphylococcal scalded skin syndrome; blistering distal dactylitis; acute paronychia; folliculitis e.g. acne vulgaris; cutaneous diphtheria; erythrasma; bacterial colonization of open wounds e.g. cuts, lesions, scrapes, burns, lacerations, chronic wounds, infected animal bites, etc.), a respiratory condition (such as pneumonia; hypersensitivity pneumonitis; upper and lower respiratory tract infections, e.g. secondary bacterial infections in chronic bronchitis and asma; chronic obstructive pulmonary disease; diphtheria; bronchopulmonary dysplasia; pertussis; legionellosis e.g. Legionnaires' disease, Pontiac fever; pharyngitis, etc.), a nasal condition (such as bacterial rhinitis; paranasal sinusitis, etc.), an ocular condition (such as chronic blepharitis; endophthalmitis, etc.), a vaginal condition (such as bacterial vaginosis; chanchroid; syphilis; donovanosis; gonorrhea; lymphogranuloma venereum; non-gonococcal urethritis; staphylococcal infection, etc.), an oral condition (such as gingivitis; dental caries; early childhood caries, etc.), a condition of the external ear (such as otitis media, etc.), a genitourinary, a rectal and other bacterial-related conditions of similar tissues.

33. The method according to claim 32, wherein the epithelial condition is acne vulgaris.

34. The method according to claim 32, wherein the epithelial condition is a respiratory condition.

35. The method according to claim 32, wherein the bacteria causing or aggravating an epithelial condition is selected from Actinobacillus sp., Actinomyces sp., Bacillus sp., Bordatella sp., Branhamella (Moraxella) sp., Calymmatobacterium sp., Chlamydia sp., Chlamydophila sp., Corynebacterium sp., Eikenella sp., Enterobacter sp., Enterococcus sp., Escherichia sp., Fusobacterium sp., Gardnerella sp., Granuloma sp., Haemophilus sp., Histophilus sp., Klebsiella sp., Legionella sp., Mannheimia sp., Mobiluncus sp., Mycobacterium sp., Mycoplasma sp., Neisseria sp., Nocardia sp., Ornithobacterium sp., Pasteurella sp., Pneumocystis sp., Prevotella sp., Propionibacterium sp., Proteus sp., Psuedomonas sp., Staphylococcus sp., Streptococcus sp., Treponema sp., Ureaplasma sp., Vibrio sp., Yersinia sp.

36. The method according to claim 35, wherein the bacteria causing or aggravating an epithelial condition is Propionibacterium acnes.

37. A method for inhibiting bacterial growth on a surface or bacterial decolonization on a surface comprising administering to a surface a complex according to claim 2.

38. A method of treating, lessening the severity of, or delaying onset of UV damage to the skin of a subject in need thereof, by administering to the subject a complex according to claim 2.

39. A method of decreasing the amount of reactive oxygen species in a cell, comprising the step of contacting the cell with a complex of according to claim 2, wherein the complex inhibits more than about 20% of superoxide formation.

40. A method of decreasing the amount of reactive oxygen species in a subject in need thereof, comprising the step of administering to the subject a complex according to claim 2, wherein the complex inhibits more than about 20% of superoxide formation.

41. The method of claim 40, further comprising administering a conventional sun-screening agent.
